(19) United States
(12) Patent Application Publication
(10) Pub. No.: US 2007/0166384 A1
(43) Pub. Date: Jul. 19, 2007

(54) Methods , composition and preparations for delivery of immune response modifiers

(76) Inventor: Isidro Angelo E. Zarraga, St. Paul, MN (US)

Correspondence Address: 3M INNOVATIVE PROPERTIES COMPANY, PO BOX 33427, ST. PAUL, MN, 55133-3427, US

(21) Appl. No.: 10/599,730

(22) PCT Filed: Apr. 8, 2005
(86) PCT No.: PCT/US05/11997

§ 371 Date: Oct. 6, 2006

Related U.S. Application Data
(60) Provisional application No. 60/560,862, filed on Apr. 9, 2004.
(60) Provisional application No. 60/617,196, filed on Oct. 8, 2004.

(51) Int. Cl. A61K 9/14; C08F 20/00

(52) U.S. Cl. 424/486; 424/488; 525/438

(57) ABSTRACT

A soluble IRM-polymer complex, preparations thereof, and methods of use, wherein the soluble IRM-polymer complex includes one or more IRM compounds attached (e.g., covalently attached) to a polymer (e.g., an alkylene oxide-containing polymer).

### CROSS-REFERENCE TO RELATED APPLICATIONS

The present application claims priority to U.S. Provisional Patent Application Ser. No. 60/560862, filed on Apr. 9, 2004, and to U.S. Provisional Patent Application Ser. No. 60/617196, filed on Oct. 8, 2004, both of which are incorporated herein by reference.

### BACKGROUND

There has been a major effort in recent years, with significant successes, to discover new drug compounds that act by stimulating certain key aspects of the immune system, as well as by suppressing certain other aspects (see, e.g., U.S. Pat. Nos. 6,039,969 and 6,200,592). These compounds, sometimes referred to as immune response modifiers (IRMs), appear to act through basic immune system mechanisms known as toll-like receptors to induce selected cytokine biosynthesis and may be used to treat a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and TH2-mediated diseases (e.g., asthma, allergic rhinitis, atopic dermatitis), and are also useful as vaccine adjuvants. Unlike many conventional anti-viral or anti-tumor compounds, the primary mechanism of action for IRMs is indirect, by stimulating the immune system to recognize and take appropriate action against a pathogen.

Many of the IRM compounds are small organic molecule imidazoquinoline amine derivatives (see, e.g., U.S. Pat. No. 4,689,338), but a number of other compound classes are now known as well (see, e.g., U.S. Pat. Nos. 5,446,153; 6,194,425; and 6,110,929) and more are still being discovered. Other IRMs have higher molecular weights, such as oligonucleotides, including CpGs (see, e.g., U.S. Pat. No. 6,194,388). In view of the great therapeutic potential for IRMs, and despite the important work that has already been done, there is a substantial ongoing need for new means of controlling the delivery and activity of IRMs in order to expand their uses and therapeutic benefits.

### SUMMARY

In some circumstances it is desirable to avoid broad systemic activity by immune response modifier (IRM) compounds (described infra), and the effectiveness of many IRMs delivered systemically may be enhanced through targeting and preferential uptake of the IRM by particular biological tissues or organs. This approach can be used to prevent, or at least reduce the occurrence of, the systemic activity of the IRM. In other words, even though the IRM can be conveniently delivered systemically, if desired, its biologic activity is concentrated at particular locations where desired.

This can be accomplished by attaching (preferably covalently attaching) one or more IRMs to an organic polymer to form a soluble complex (herein referred to as a soluble IRM-polymer complex). That is, a soluble IRM-polymer complex of the present invention is of a size and chemical nature to allow preferential deposition in certain tissues (e.g., particular tissue types and/or localized tissue regions) such as solid tumors, lymph tissue, reticuloendothelial system, bone marrow, mucosal tissue, etc.

Typically, the polymer of the soluble IRM-polymer complex is also soluble prior to attachment of one or more IRMs. Preferably, the polymer (i.e., polymer carrier material) includes alkylene oxide (e.g., ethylene oxide) moieties. Such polymers are referred to herein as “alkylene oxide-containing polymers.”

In this context, in certain embodiments, “soluble” refers to a polymer IRM-complex (and/or, typically, the polymer prior to attachment of the one or more IRMs) having a solubility of at least 1 microgram per milliliter in water under physiological conditions (i.e., pH 7.4 and 37° C.). In certain embodiments, the polymer-IRM complex (and/or the polymer prior to attachment of the one or more IRMs) has a solubility of at least 0.1 microgram per milliliter in water under physiological conditions. In certain embodiments, the polymer-IRM complex (and/or the polymer prior to attachment of the one or more IRMs) has a solubility of at least 0.1 and less than 1 microgram per milliliter in water under physiological conditions.

The IRM can be biologically active while attached (preferably, covalently attached) to the polymer (preferably, polyalkylene oxide-containing polymer), although this is not a necessary requirement of the invention. For example, the IRM may be “inactive” due to masking of its activity by folding of the polymer carrier material around the IRM or due to the IRM-polymer linkage to a position on the IRM required for IRM activity. Once the soluble IRM-polymer complex has reached a targeted site, the IRM can detach from the polymer carrier material (preferably, polyalkylene oxide-containing carrier material) (e.g., through biodegradation of the polymer-IRM bond or unfolding of the polymer carrier material), thereby resulting in availability or activation of the IRM. Other mechanisms of activation of the IRM may also occur once the soluble IRM-complex has reached a targeted site.

Accordingly, the invention includes a method of providing an IRM compound to a targeted tissue region (e.g., a localized tissue region and/or tissue type (i.e., cell type)) using a soluble IRM-polymer complex disclosed herein. The IRM localized tissue region may be, e.g., a cancer, a viral infected lesion, or organ, or vaccination site. It may be a solid tumor, lymph tissue, reticuloendothelial system, bone marrow, mucosal tissue, etc. The localized tissue region may be, e.g., a breast cancer tumor, stomach cancer tumor, lung cancer tumor, head or neck cancer tumor, colorectal cancer tumor, renal cell carcinoma tumor, pancreatic cancer tumor, basal cell carcinoma tumor, pancreatic cancer tumor, cervical cancer tumor, melanoma cancer tumor, prostate cancer tumor, ovarian cancer tumor, or bladder cancer tumor.

The IRM may be an agonist of at least one TLR selected from the group consisting of TLR7, TLR8, and combinations thereof. The IRM may be a selective TLR agonist of TLR 7, or TLR 8, or an agonist of both TLR 7 and 8. The IRM may preferably be a small molecule immune response modifier, for example, comprising a 2-aminopyridine fused to a five-membered nitrogen-containing heterocyclic ring.

In one embodiment, the present invention provides a method of delivering one or more IRM compounds to a tissue in a subject, the method involves administering (preferably, systemically administering) an IRM preparation to the subject, wherein the IRM preparation includes a soluble IRM-polymer complex including one or more IRM compounds attached to a polymer.

Herein, in certain embodiments, a soluble IRM-polymer complex is one that has a solubility in water of at least 1 microgram per milliliter under physiological conditions. In certain embodiments, the IRM-polymer complex has a solubility of at least 0.1 microgram per milliliter in water under physiological conditions, and in certain embodiments, a solubility of at least 0.1 and less than 1 microgram per milliliter in water under physiological conditions. In certain embodiments, the IRM-polymer complex has a solubility in water of at least 10 micrograms per milliliter under physiological conditions. In certain embodiments, the IRM-polymer complex has a solubility in water of at least 100 micrograms per milliliter under physiological conditions.

Preferably, the one or more IRM compounds are covalently attached to the polymer. Preferably, the polymer is soluble prior to attachment of the one or more IRM compounds. That is, in certain embodiments, the polymer prior to attachment of the one or more IRM compounds preferably has a solubility in water of at least 1 microgram per milliliter under physiological conditions. In certain embodiments, the polymer prior to attachment of the one or more IRM compounds has a solubility of at least 0.1 microgram per milliliter in water under physiological conditions, and in certain embodiments, a solubility of at least 0.1 and less than 1 microgram per milliliter in water under physiological conditions. In certain embodiments, the polymer prior to attachment of the one or more IRM compounds has a solubility in water of at least 10 micrograms per milliliter under physiological conditions. In certain embodiments, the polymer prior to attachment of the one or more IRM compounds has a solubility in water of at least 100 micrograms per milliliter under physiological conditions.

The polymer can be selected from the group consisting of poly(alkylene glycols), poly(olefinic alcohols), polyvinylpyrrolidones, poly(hydroxyalkylmethacrylamides), poly(hydroxyalkylmethacrylates), polyvinyl alcohols, polyoxazolines, poly(acrylic acids), polyacrylamides, polyglutamates, polylysines, polysaccharides, and combinations thereof. In certain embodiments, the polymer includes alkylene oxide moieties.

In another embodiment, the present invention provides a method of delivering one or more IRM compounds to a tissue in a subject, wherein the method includes administering (preferably, systemically administering) an IRM preparation to the subject, wherein the IRM preparation includes a soluble IRM-polymer complex including one or more IRM compounds attached to a soluble polymer having alkylene oxide moieties, wherein the IRM-polymer complex has a molecular weight of 1 kDa to 500 kDa, and in certain embodiments 1 kDa to 200 kDa.

The polymer (and/or the IRM-polymer complex) typically can have a molecular weight of at least 1 kDa, or at least 20 kDa, or at least 30 kDa. The polymer (and/or the IRM-polymer complex) typically can have a molecular weight of no greater than 500 kDa, or no greater than 200 kDa, or no greater than 100 kDa, or no greater than 50 kDa. The polymer (and/or the IRM-polymer complex) can have a molecular weight of 1 kDa to 200 kDa, or 1 kDa to 100 kDa, or 1 kDa to 50 kDa. In certain embodiments, the polymer (and/or the IRM-polymer complex) can have a molecular weight of 1 kDa to 500 kDa, or 20 kDa to 200 kDa, or 30 kDa to 100 kDa.

The present invention also provides a soluble IRM-polymer complex that includes one or more IRM compounds attached to a polymer. In certain embodiments, the polymer prior to attachment of the one or more IRM compounds has a solubility in water of at least 1 microgram per milliliter under physiological conditions. In certain embodiments, the polymer prior to attachment of the one or more IRM compounds has a solubility of at least 0.1 microgram per milliliter in water under physiological conditions, and in certain embodiments, a solubility of at least 0.1 and less than 1 microgram per milliliter in water under physiological conditions. In certain embodiments the polymer includes alkylene oxide-containing moieties.

IRM preparations are also provided that include one or more soluble IRM-polymer complexes as defined herein. Such preparations can also include one or more additional active agents, which may or may not be attached to the polymer. For example, a preparation can include one or more IRM compounds that are not attached to the polymer.

Herein, “polymer” is used to encompass homopolymers and copolymers, “copolymer” is used to encompass polymers prepared from two or more different monomers (e.g., terpolymers, tetrapolymers, etc.).

The term “comprises” and variations thereof do not have a limiting meaning where these terms appear in the description and claims.

As used herein, “a,” “an,” “the,” “at least one,” and “one or more” are used interchangeably. Thus, for example, a complex that comprises “an” IRM can be interpreted to mean that the complex includes “one or more” IRMs. Similarly, a composition comprising “a” complex can be interpreted to mean that the composition includes “one or more” complexes.

As used herein, “treating” a condition or a subject includes therapeutic, prophylactic, and diagnostic treatments.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used individually and in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention is directed to methods, complexes, and preparations (i.e., compositions or formulations) of immune response modifiers (IRMs) that can be preferentially targeted to a localized tissue region and/or tissue type and/or provide locally (or systemically) active IRM compounds for an extended period of time. Such complexes include a polymer carrier material having one or more IRM compounds attached thereto.

A soluble IRM-polymer complex of the present invention is of a size and chemical nature to allow preferential deposition in certain tissues (e.g., particular tissue types and/or localized tissue regions) such as solid tumors, lymph tissue, reticuloendothelial system, bone marrow, mucosal tissue, etc. Such IRM-polymer complexes are soluble in water (i.e., for certain embodiments at least 1 microgram per milliliter, and for certain embodiments at least 0.1 microgram per milliliter) under physiological conditions. Due to the solubility of the IRM-polymer complex, one advantage of the present invention is that the circulatory system can be used to quickly distribute the complex throughout the body. Also, a clear or semi-clear solution of the soluble IRM-polymer complex may be more easily administered to a patient than a formulation that includes particulates, emulsions, or other constructs.

Another advantage can be described in terms of the IRM half-life. To illustrate, if a conventional solution formulation of a given IRM compound is injected systemically, the IRM compound has a short half-life and is quickly removed via renal excretion. By contrast, if a soluble IRM-polymer complex such as those described herein is injected systemically the large molecular weight of the IRM-polymer complex overcomes renal excretion, increasing the half-life of the IRM.

The present invention thus provides active IRMs accumulated within a localized tissue region and/or tissue type in an amount greater than and/or for a time longer than a comparable concentration of the IRM in a conventional solution. For example, the tissue concentration for the IRM when administered as an IRM-polymer complex is preferably at least 50% greater than the localized tissue concentration for an uncomplexed IRM when administered in a similar manner. For example, the residence half-life for the IRM when administered as an IRM-polymer complex is preferably at least 50% greater than the residence half life of an uncomplexed IRM.

Polymers for use in the soluble IRM-polymer complexes may be sufficiently flexible in water to mask or hide an active IRM from the immune system preventing or reducing a systemic response and local response at the administration site (typically, by preventing or reducing immune cell receptors from attaching to the IRM). It is believed that unfolding and/or biodegradation of the polymer will make the IRM available for stimulating an immune response. Alternatively, the polymer can be less flexible so that it does not envelop the IRM, in which case, depending on the attachment site of the polymer on the IRM, the IRM may be active while it is still attached to the polymer.

The flexibility and solubility of preferred IRM-polymer complexes of the present invention are believed to allow for temporal fluctuations in polymer conformation, thereby preventing, or reducing the occurrence of immune cell receptors from latching on to a fixed molecular structure. Although not intending to be limiting, this is believed to contribute to the complex remaining inactive until the target site is reached, thereby potentially reducing systemic side effects of IRMs.

Furthermore, the enhanced permeability and retention (EPR) effect in tumor vasculature is believed to facilitate extravasation of the IRM-polymer complex selectively at the tumor site and allow it to accumulate therein (see, e.g., Hiroshi Maeda, Advanced Drug Delivery Reviews, 6(2): 181-202, (1991)).

Additionally, the IRM-polymer complex can be designed, e.g., by attaching a particular antibody to the complex, to target and bind to tumor antigens present at the tumor or in the circulatory system, thereby inducing a more potent immune response. In this fashion, the IRM-polymer-antibody complex could induce an immune response targeted to the tumor antigen.

Also, accumulation of a soluble IRM-polymer complex in the targeted tissue may cause inflammation that could attract effector and/or memory T cells into the area.

Another advantage of the present invention is to ‘protect’ the IRM from immune cells and thus avoid or reduce the generation of antibodies against the IRM and eliminate potential allergic responses to the IRM pharmacophore.

The benefits of the present invention in terms of improved targeting of the immune system, with reduced systemic activity, can be accomplished with many different soluble IRM-polymer complexes, optionally with other active agents, and can be targeted to various localized tissue regions and/or tissue types for a wide range of treatments.

Soluble IRM-Polymer Complexes and Preparations Thereof

As described above, a soluble IRM-polymer complex (and preparations and compositions thereof) can provide active IRM compound, after delivery (preferably systemic delivery), for an extended period to a localized tissue region and/or tissue type, while reducing overall systemic activity of the IRM.

This can be accomplished by attaching (preferably covalently attaching) one or more IRMs to an organic polymer to form a soluble complex (herein referred to as a soluble IRM-polymer complex). That is, a soluble IRM-polymer complex of the present invention is of a size and chemical nature to allow preferential deposition in tissues (e.g., particular tissue types or localized tissue regions) such as solid tumors. This can occur as a result of the tissue's increased vascular permeability, for example, to soluble IRM-polymer complexes of the present invention, and the reduced lymphatic drainage of tumor tissues.

Typically, the polymer of the soluble IRM-polymer complex is also soluble prior to attachment of one or more IRMs. Preferably, the polymer (i.e., polymer carrier material) includes alkylene oxide (e.g., ethylene oxide) moieties. Such polymers are referred to herein as “alkylene oxide-containing polymers.”

In this context, in certain embodiments, “soluble” refers to an IRM-polymer complex having a solubility of at least 1 microgram per milliliter in water under physiological conditions (i.e., pH 7.4 and 37° C.). Typically, prior to attachment, the polymer of the IRM-polymer complex has a solubility of at least 1 microgram per milliliter in water under physiological conditions (i.e., pH 7.4 and 37° C.). In certain embodiments, however, an IRM-polymer complex has a solubility of at least 0.1 microgram per milliliter in water under physiological conditions (i.e., pH 7.4 and 37° C.). In certain embodiments, prior to attachment, the polymer of the IRM-polymer complex has a solubility of at least 0.1 microgram per milliliter in water under physiological conditions (i.e., pH 7.4 and 37° C.). In certain embodiments, an IRM-polymer complex, and/or the polymer prior to attachment of an IRM, has a solubility of at least 0.1 and less than 1 microgram per milliliter in water under physiological conditions.

For certain embodiments, the IRM-polymer complex, and/or the polymer prior to attachment of an IRM, has a solubility of at least 10 micrograms per milliliter in water under physiological conditions. For certain embodiments, the IRM-polymer complex, and/or the polymer prior to attachment of an IRM, has a solubility of at least 100 micrograms per milliliter in water under physiological conditions.

As long as the IRM-polymer complex is sufficiently soluble, the complex (and the polymer prior to attachment of one or more IRMs) can be of a wide variety of molecular weights. Preferably, the complex (and/or the polymer prior to attachment of one or more IRMs) has a molecular weight of at least 1 kilodalton (kDa). More preferably, the complex (and/or the polymer prior to attachment of one or more IRMs) has a molecular weight of at least 20 kDa. Even more preferably, the complex (and/or the polymer prior to attachment of one or more IRMs) has a molecular weight of at least 30 kDa. Preferably, the complex (and/or the polymer prior to attachment of one or more IRMs) has a molecular weight of no greater than 500 kilodaltons (kDa). More preferably, the complex (and/or the polymer prior to attachment of one or more IRMs) has a molecular weight of no greater than 200 kDa. Even more preferably, the complex (and/or the polymer prior to attachment of one or more IRMs) has a molecular weight of no greater than 100 kDa, and often no greater than 50 kDa.

Suitable polymers for attachment (preferably covalent attachment) to an IRM include poly(alkylene glycols) (i.e., polyalkylene oxides) such as poly(oxyethylated polyols), poly(olefinic alcohols), polyester polyols, polyvinylpyrrolidones, poly(hydroxyalkylmethacrylamides), poly(hydroxyalkylmethacrylates), polyvinyl alcohols, polyoxazolines (e.g., polyethyloxazoline), poly(acrylic acids) (typically, those that are not crosslinked), polyacrylamides, polyglutamates, polylysines, polysaccharides, and combinations thereof (e.g., copolymers, terpolymers, etc., and mixtures thereof). Preferably, suitable polymers are those within these classes that are soluble (i.e., have a solubility of at least 1 microgram per milliliter in water under physiological conditions, and in certain embodiments, have a solubility of at least 0.1 microgram per milliliter in water under physiological conditions). Particularly suitable polymers within these classes of polymers are those that have a solubility of at least 10 micrograms per milliliter in water under physiological conditions, and often at least 100 micrograms per milliliter in water under physiological conditions.

Examples of preferred aqueous soluble polymers include polyvinyl alcohols, polyacrylamides, polyalkylene oxides (e.g., polyethylene oxide), poly(hydroxyalkylmethacrylamides) (e.g., poly N-(2-hydroxypropyl)methacrylamide), polyglutamates, polylysines, polysaccharides (e.g., cellulose (e.g., carboxymethyl cellulose, hydroxypropylmethyl cellulose), starch, dextran amylose, glycogen, chitin, etc.), and combinations thereof (e.g., copolymers and mixtures thereof). Particularly preferred polymers include alkylene oxide (preferably, ethylene oxide) moieties.

A preferred class of aqueous soluble polymers include poly(alkylene oxide)polymers that include C₂-C₄ alkylene oxide moieties, particularly the following alkylene oxide moieties: 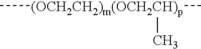
wherein m is at least 2 (and more preferably, at least 25) and p is 0 to 9,000 (and, in certain embodiments 0 to 5,000, in certain embodiments, 0 to 1,000, and in certain embodiments, 0 to 50). In this representation, the isopropylene oxide groups (the “p” groups) and the ethylene oxide groups (the “m” groups) can be arranged in a reversed, alternating, random, or block configuration. In any one polymer, m is preferably at least 4 (more preferably, at least 25, even more preferably, at least 450, and even more preferably, at least 700). Preferably, m is no greater than 12,000 (more preferably, no greater than 5000, even more preferably, no greater than 2,500, even more preferably, no greater than 1,000, even more preferably, no greater than 115, even more preferably, no greater than 45, and even more preferably, no greater than 25). Preferably, p is 0.

Commercially available polyethylene glycols (PEG) include those having backbones of the formulas HO—(CH₂CH₂O)ₙ—CH₂CH₂—OH (PEG) and CH₃O—(CH₂CH₂)ₙ—CH₂CH₂—OH (mPEG), which are modified for attachment of one or more IRMs. Specific materials that are commercially available include, but are not limited to, ACRL-PEG-NHS, Biotin-PEG-NHS, Boc-Protected Amine, Fluorescein-PEG-NHS, Fmoc-Protected Amine, NHS-PEG-Maleimide, NHS-PEG-Vinylsulfone, mPEG-Acetaldehyde Diethyl Acetal, mPEG-Benzotriazole Carbonate, mPEG-ButyrALD, mPEG-Double Esters, mPEG-DSPE, mPEG-Forked Maleimide, mPEG-Maleimide, mPEG-NH2, mPEG-Succinimidyl Butanoate, mPEG-Succinimidyl Propionate, mPEG-Thioesters, mPEG2-Aldehyde, mPEG2-ButyrALD, mPEG2-Forked Maleimide, mPEG2-N-Hydroxylsuccinimide, mPEG2-Maleimide, Multi-Arm PEGs and raw PEGs (all of which are available from Nektar Therapeutics, San Carlos, Calif.).

An IRM can be linked to a polymer with charged regions (+ or −) that enhance electrostatically favorable attachment of the IRM-polymer complex to antigens (e.g., expressed on cancer cell surfaces). Typically, under physiological conditions positively charged polymer-IRM complexes will bind to antigens with isoelectric points (pI) below 7, and negatively charged polymer-IRM complexes will bind to antigens with pIs above 7.

A mixture of IRMs linked to different molecular weights of polymer (and/or different polymers) may also achieve a desired release profile, and may be a way to influence the time course of immune response. For example, a pulsed release profile of an IRM, with 2-3 day spacing, can be therapeutically beneficial. Such a pulsed release of an IRM can avoid (or at least reduce the occurrence of) hyposensitization, local inflammation, and/or tolerance to treatment, while allowing dendritic cells enough time to be replenished by naïve ones at the site of a tumor, for example.

One or more IRMs can be attached to a polymer through either covalent attachment or non-covalent attachment. Non-covalent attachment of an IRM to a polymer carrier material includes attachment by ionic interaction or hydrogen bonding, for example.

Representative methods for covalently attaching an IRM to a polymer include chemical crosslinkers, such as heterobifunctional crosslinking compounds (i.e., “linkers”) that react to form a bond between reactive groups (such as hydroxyl, amino, amido, or sulfhydryl groups) in an immune response modifier and other reactive groups (of a similar nature) in the polymer. This bond may be, for example, a peptide bond, disulfide bond, thioester bond, amide bond, thioether bond, and the like. IRMs can also be covalently attached to a polymer by reacting an IRM containing a reactive group directly with a polymer containing a reactive group.

Immune response modifiers may be covalently bonded to a polymer by any of the methods known in the art. For example, U.S. Pat. Nos. 4,722,906, 4,979,959, 4,973,493, and 5,263,992 relate to devices having biocompatible agents covalently bound via a photoreactive group and a chemical linking moiety to the biomaterial surface. U.S. Pat. Nos. 5,258,041 and 5,217,492 relate to the attachment of biomolecules to a surface through the use of long chain chemical spacers. U.S. Pat. Nos. 5,002,582 and 5,263,992 relate to the preparation and use of polymeric surfaces, wherein polymeric agents providing desirable properties are covalently bound via a photoreactive moiety to the surface.

In one embodiment, the IRM can be attached to a polymer using a linking group. The linking group can be any suitable organic linking group that allows the polymer to be covalently coupled to the immune response modifier moiety while preserving an effective amount of IRM activity. In some embodiments, the linker group can be a hydrolysable linker, enzymatic specific linker, or a protease specific linker. In some embodiments, the linking group may be selected to create sufficient space between the active core of the immune response modifier moiety and the polymer that the polymer does not interfere with a biologically effective interaction between the active core and the T cells that results in IRM activity such as cytokine production.

In this embodiment, the linking group includes a reactive group capable of reacting with a reactive group on the polymer to form a covalent bond. Suitable reactive groups include those discussed in Hermanson, G. (1996), Bioconjugate Techniques, Academic Press, Chapter 2 “The Chemistry of Reactive Functional Groups”, 137-166. For example, the linking group may react with a primary amine (e.g., an N-hydroxysuccinimidyl ester or an N-hydroxysulfosuccinimidyl ester); it may react with a sulfhydryl group (e.g., a maleimide or an iodoacetyl), or it may be a photoreactive group (e.g. a phenyl azide including 4-azidophenyl, 2-hydroxy-4-azidophenyl, 2-nitro-4-azidophenyl, and 2-nitro-3-azidophenyl).

In this embodiment, the polymer includes a chemically active group accessible for covalent coupling to the linking group. A chemically active group accessible for covalent coupling to the linking group includes groups that may be used directly for covalent coupling to the linking group or groups that may be modified to be available for covalent coupling to the linking group. For example, suitable chemically active groups include, but are not limited to, primary amines and sulfhydryl groups.

In certain embodiments, attachment may occur by reacting an immune response modifier with a crosslinker and then reacting the resulting intermediate with a polymer. Many crosslinkers suitable for such use are known and many are commercially available. See for example, Hermanson, G. (1996) Bioconjugate Techniques, Academic Press.

Attachment also may occur, for example, according to the method of Reaction Scheme I in which the polymer is linked to the IRM moiety through R₁₁. In Reaction Scheme I an IRM of Formula II is reacted with a polymer of Formula III to provide an IRM-polymer complex of Formula I. R_{A} and R_{B} each contain a functional group that is selected to react with the other. For example, if R_{A} contains a primary amine, then a polymer may be selected in which R_{B} contains an amine-reactive functional group such as an N-hydroxysuccinimidyl ester. R_{A} and R_{B} may be selected so that they react to provide the desired linker group in the IRM-polymer complex.

Methods for preparing compounds of Formula II where R_{A} contains a functional group are known. See, for example, U.S. Pat. Nos. 4,689,338; 4,929,624; U.S. Pat. Nos. 5,268,376; 5,389,640; 5,352,784; 5,494,916; 4,988,815; 5,367,076; 5,175,296; 5,395,937; 5,741,908; 5,693,811; 6,069,149; 6,194,425; 6,331,539; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; U.S. Patent Publications 2004/0147543 and 2004/0176367; and International Publication WO 03/103584. Many polymers containing R_{B} groups are known and many are commercially available. For example, activated polyethylene glycols available from Nektar, San Carlos, Calif. Others can be prepared using known synthetic methods. See, for example, U.S. Pat. No. 5,583,114 and the references cited therein.

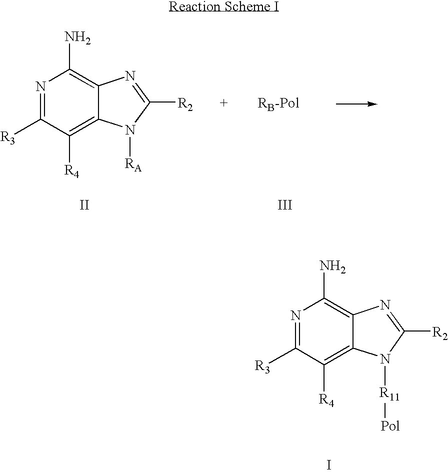

The R groups (e.g., R₁, R₂, R₃, and R₄) can be hydrogen or organic groups that can optionally include various substitutions. They can include alkyl groups, alkenyl groups, including haloalkyl groups, aryl groups, heteroaryl groups, heterocyclyl groups, and the like.

For example, preferred R₁ groups include, alkyl groups having 1 to 4 carbon atoms, hydroxyalkyl groups having 1 to 4 carbon atoms (e.g., 2-hydroxy-2-methylpropyl), methanesulfonylaminoalkyl groups wherein the alkyl group has 2 to 6 carbons (e.g. methanesulfonylaminobutyl, 2-methanesulfonylamino-2-methylpropyl); preferred R₂ groups include hydrogen, alkyl groups having 1 to 4 carbon atoms (i.e., methyl, ethyl, propyl, isopropyl, n-butyl, sec-butyl, isobutyl, tert-butyl, and cyclopropylmethyl), and alkoxyalkyl groups (e.g., methoxyethyl and ethoxymethyl). Preferably R₃ and R₄ are independently hydrogen or methyl or R₃ and R₄ join together to form a benzene ring, a pyridine ring, a 6-membered saturated ring or a 6-membered saturated ring containing a nitrogen atom. One or more of these preferred substituents, if present, can be present in the compounds of the invention in any combination.

In Reaction Scheme I the IRM is attached to the polymer through a linking group at the N1 nitrogen of the imidazole ring. Alternatively the linking can occur at different positions on the ring system. Examples of which are shown below for imidazoquinoline amines, imidazonaphthyridine amines and imidazopyridine amines respectively. 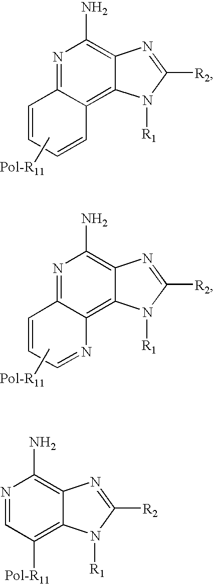
The attachment is effected using the method of Reaction Scheme I starting with an IRM containing reactive group R_{A} at the desired attachment point. In another embodiment, the polymer group can be attached to the 4-amino group of an IRM. Attachment may occur, for example, using a variation of the method of Reaction Scheme I by reacting an IRM with R_{B}-polymer where R_{B} contains an amine-reactive functional group. Attachment may also occur using the methods described in Reaction Schemes II, III, IV, and V below.

In Reaction Scheme II, a polyethylene glycol polymer is attached to an IRM by the formation of an amide with the 4-amino group of the IRM. The reaction can be carried out by adding a succinimidyl propionate of Formula V to a solution of an IRM of Formula IV in a suitable solvent such as tetrahydrofuran. The reaction can be carried out at ambient temperature or at an elevated temperature such as 50° C. Some succinimidyl propionates of Formula V are commercially available; others can be prepared using conventional synthetic methods. Many IRMs of Formula IV are known (see Exemplary IRM Compounds below); preferably compounds wherein the R₁, R₂, R₃, and R₄ groups do not contain a primary amine are selected.

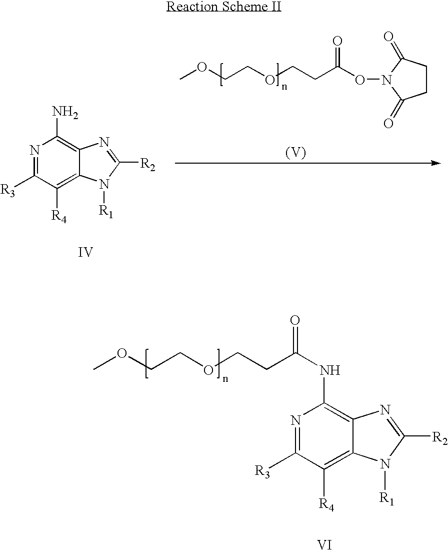

In Reaction Scheme III, a polyethylene glycol polymer is end capped with an IRM of Formula IV.

In step (1) of Reaction Scheme III, a polyethylene glycol polymer of Formula VII is reacted with phosgene to provide a bischloroformate of Formula VIII. The reaction can be carried out by treating a solution of a polymer of Formula VII in a suitable solvent such as toluene with an excess of phosgene. The reaction can be run at an elevated temperature such as about 45° C.

In step (2) of Reaction Scheme III, a bischloroformate of Formula VIII is reacted with pentafluorophenol to provide an activated carbonate of Formula IX. The reaction can be carried out by adding pentafluorophenol to a solution of a compound of Formula VIII in a suitable solvent such as toluene in the presence of a base such as triethylamine.

In step (3) of Reaction Scheme III, an activated carbonate of Formula IX is reacted with an IRM of Formula IV to provide an IRM substituted polyethylene glycol polymer of Formula X. The reaction can be carried out by treating a solution of a compound of Formula IX in a suitable solvent such as isopropanol with an IRM of Formula IV.

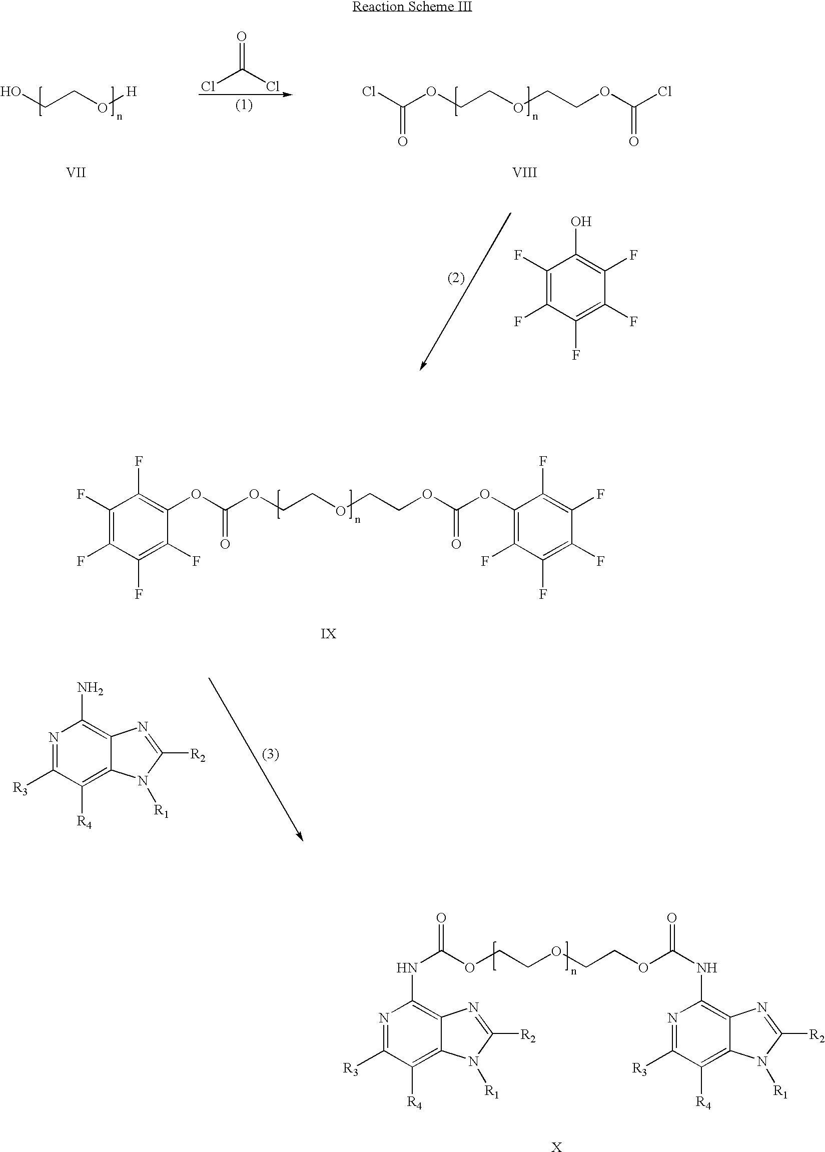

In Reaction Scheme IV, a polyethylene glycol polymer is chain extended with an IRM of Formula IV. The reaction can be carried out by adding m equivalents of a bischloroformate of Formula VIII to a solution containing m+1 equivalents of an IRM of Formula IV in a suitable solvent such as tetrahydrofuran in the presence of a base such as triethylamine. The reaction scheme illustrates 2 moles of a bischloroformate of Formula VIII reacting with 3 moles of an IRM of Formula IV

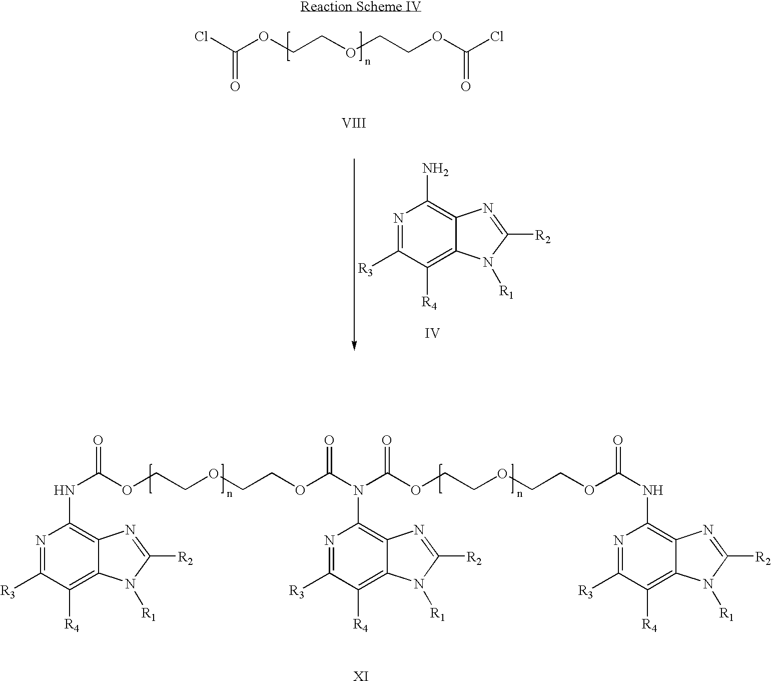

Reaction Scheme V illustrates the preparation of an IRM substituted multivalent polyethylene glycol polymer.

In step (1) of Reaction Scheme V, (2,5-diethyl-2-methyl-1,3-dioxan-5-yl)methanol is treated with phosgene to provide (2,5-diethyl-2-methyl-1,3-dioxan-5-yl)methyl chloridocarbonate. The reaction can be carried out by treating a solution of (2,5-diethyl-2-methyl-1,3-dioxan-5-yl)methanol in a suitable solvent such as toluene with phosgene.

In step (2) of Reaction Scheme V, (2,5-diethyl-2-methyl-1,3-dioxan-5-yl)methyl chloridocarbonate is reacted with pentafluorophenol to provide (2,5-diethyl-2-methyl-1,3-dioxan-5-yl)methyl pentafluorophenyl carbonate. The reaction can be carried out by adding pentafluorophenol to a solution of (2,5-diethyl-2-methyl-1,3-dioxan-5-yl)methyl chloridocarbonate in a suitable solvent such as tetrahydrofaran in the presence of a base such as pyridine.

In step (3) of Reaction Scheme V, (2,5-diethyl-2-methyl-1,3-dioxan-5-yl)methyl pentafluorophenyl carbonate is hydrolyzed under acidic conditions using conventional methods to provide 2,5-bis(hydroxymethyl)butyl pentafluorophenyl carbonate.

In step (4) of Reaction Scheme V, a bischloroformate of Formula VIII is reacted with 2,5-bis(hydroxymethyl)butyl pentafluorophenyl carbonate to provide a polyethylene glycol polymer of Formula XII containing activated carbonate groups. The reaction can be carried out as described in step (2) of Reaction Scheme III.

In step (5) of Reaction Scheme V, a polyethylene glycol polymer of Formula XII is reacted with an IRM of Formula IV to provide an IRM substituted multivalent polyethylene glycol polymer of Formula XIII. The reaction can be carried out as described in step (3) of Reaction Scheme III. 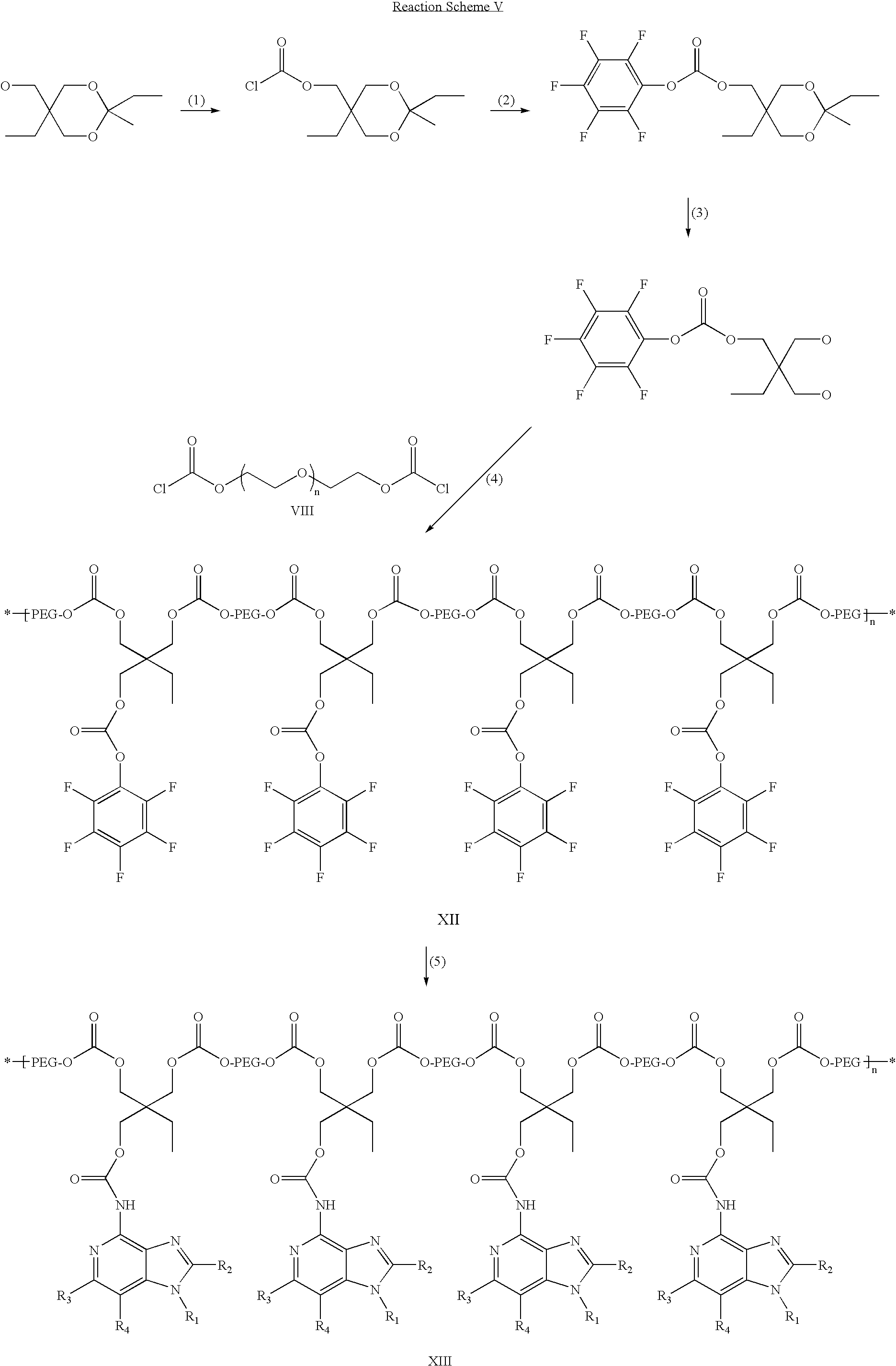
Delivery of IRM-Polymer Complexes

The IRM preparations may be delivered via parenteral administration (by definition parenteral administration refers to non-oral administration, which would include nasal, topical, ophthalmic, buccal, etc., but in practice usually refers to injectable products (intravenous, intramuscular, subcutaneous, intratumoral, etc.) using, e.g., needle injection, injection using a microneedle array, or any other known method for introducing a preparation parenterally. Once it is administered, the soluble IRM-polymer complex will typically automatically target a localized tissue region and/or tissue type (i.e., cell type). Delivery of the soluble IRM-polymer complex may be in conjunction with image guiding techniques using, for example, ultrasound, MRI, real-time X-ray (fluoroscopy), etc.

A “localized tissue region” will generally be a relatively small portion of the body, e.g., less than 10% by volume, and often less than 1% by volume. For example, depending on the size of, e.g., a solid tumor or cancerous organ, the localized tissue region will typically be on the order of no more than about 500 cm³, often less than about 100 cm³, and in many instances 10 cm³ or less. For some applications the localized tissue region will be 1 cm³ or less (e.g., for small tumor nodules, viral lesions, or vaccination sites). However, in certain instances the localized tissue region may be a particularly large region, up to several liters, for example, to treat metastasized cancer within the entire peritoneal cavity.

The IRM localized tissue region may be, e.g., a cancer, a viral infected lesion, or organ, or vaccination site. It may be a solid tumor, lymph tissue, reticuloendothelial system, bone marrow, mucosal tissue, etc. The localized tissue region may be, e.g., a breast cancer tumor, stomach cancer tumor, lung cancer tumor, head or neck cancer tumor, colorectal cancer tumor, renal cell carcinoma tumor, pancreatic cancer tumor, basal cell carcinoma tumor, pancreatic cancer tumor, cervical cancer tumor, melanoma cancer tumor, prostate cancer tumor, ovarian cancer tumor, or bladder cancer tumor.

Additional Agents

In addition to one or more soluble IRM-polymer complexes, the IRM preparations (i.e., compositions) and methods of the present invention can include additional agents (particularly active agents), e.g., in admixture or administered separately. The additional agents can also be attached to the IRM-polymer complex (e.g., an antibody can be attached to the polymer or an IRM-antigen conjugate can be attached to the polymer).

Such additional agents may be additional active agents, including, for example, a chemotherapeutic agent, a cytotoxoid agent, an antibody, a cytokine, a vaccine or a tumor necrosis factor receptor (TNFR) agonist. One or more IRMs that are not attached to the polymer carrier material can also be included.

Vaccines include any material that raises either humoral and/or cell mediated immune response, such as live or attenuated viral and bacterial immunogens and inactivated viral, tumor-derived, protozoal, organism-derived, fungal, and bacterial immunogens, toxoids, toxins, polysaccharides, proteins, glycoproteins, peptides, cellular vaccines, such as using dendritic cells, DNA vaccines, recombinant proteins, glycoproteins, and peptides, and the like, for use in connection with, e.g., cancer vaccines, BCG, cholera, plague, typhoid, hepatitis A, B, and C, influenza A and B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, severe acute respiratory syndrome (SARS), anthrax, and yellow fever. See also, e.g., vaccines disclosed in International Publication No. WO 02/24225. Such additional agents can include, but are no limited to, drugs, such as antiviral agents or cytokines. The vaccine may be separate or may be physically or chemically linked to the IRM, such as by chemical conjugation or other means, so that they are delivered as a unit. TNFR agonists that may be delivered in conjunction with the IRM preparation include, but are not limited to, CD40 receptor agonists, such as disclosed in copending application U.S. Patent Publication 2004/0141950. Other active ingredients for use in combination with an IRM preparation of the present invention include those disclosed in, e.g., U.S. Patent Publication No. 2003/0139364.

Immune Response Modifier Compounds

Immune response modifiers (“IRM”) useful in the present invention include compounds that act on the immune system by inducing and/or suppressing cytokine biosynthesis. IRM compounds possess potent immunostimulating activity including, but not limited to, antiviral and antitumor activity, and can also down-regulate other aspects of the immune response, for example shifting the immune response away from a TH-2 immune response, which is useful for treating a wide range of TH-2 mediated diseases. IRM compounds can also be used to modulate humoral immunity by stimulating antibody production by B cells. Further, various IRM compounds have been shown to be useful as vaccine adjuvants (see, e.g., U.S. Pat. Nos. 6,083,505 and 6,406,705, and International Publication No. WO 02/24225).

In particular, certain IRM compounds effect their immunostimulatory activity by inducing the production and secretion of cytokines such as, e.g., Type I interferons, TNF-α, IL-1, IL-6, IL-8, IL-10, IL-12, MIP-1, MIP-3alpha and/or MCP-1, and can also inhibit production and secretion of certain TH-2 cytokines, such as IL-4 and IL-5. Some IRM compounds are said to suppress IL-1 and TNF (U.S. Pat. No. 6,518,265).

For some embodiments, the preferred IRM compounds are so-called small molecule IRMs, which are relatively small organic compounds (e.g., molecular weight under about 1000 daltons, preferably under about 500 daltons, as opposed to large biologic protein, peptides, and the like). Although not bound by any single theory of activity, some IRMs are known to be agonists of at least one Toll-like receptor (TLR). IRM compounds that are agonists for TLRs selected from 7 and/or 8 may be particularly useful for certain applications. In some applications, for example, the preferred IRM compound is not a TLR 7 agonist and is a TLR 8 agonist. In other applications, for example, the IRM is a TLR7 agonist and is not a TLR8 agonist. Some small molecule IRM compounds are agonists of TLRs such as 7 and/or 8 and perhaps others. Thus, in some embodiments, the IRM that is included in the soluble IRM-polymer complex may be a compound identified as an agonist of one or more TLRs.

For example, without being bound to any particular theory or mechanism of action, IRM compounds that activate a strong cytotoxic lymphocyte (CTL) response may be particularly desirable as vaccine adjuvants, especially for therapeutic viral and/or cancer vaccines because a therapeutic effect in these settings is dependent on the activation of cellular immunity. For example, studies have shown that activation of T cell immunity in a given patient has a significant positive effect on the prognosis of the patient. Therefore the ability to enhance T cell immunity is believed to be critical to producing a therapeutic effect in these disease settings.

IRM compounds that are TLR8 agonists may be particularly desirable for use with therapeutic cancer vaccines because antigen presenting cells that express TLR8 have been shown to produce IL-12 upon stimulation through TLR8. IL-12 is believed to play a significant role in activation of CTLs, which are important for mediating therapeutic efficacy as described above.

IRM compounds that are TLR7 agonists may be particularly desirable for use with prophylactic vaccines because the type I interferon induced by stimulation through these TLRs is believed to contribute to the formation of neutralizing Th1-like humoral and cellular responses.

IRM compounds that are both TLR7 and TLR8 agonists may be particularly desirable for use with therapeutic viral vaccines and/or cancer vaccines because TLR7 stimulation is believed to induce the production of type I IFN and activation of innate cells such as macrophages and NK cells, and TLR8 stimulation is believed to activate antigen presenting cells to initiate cellular adaptive immunity as described above. These cell types are able to mediate viral clearance and/or therapeutic growth inhibitory effects against neoplasms.

IRM compounds that are non-TLR7 agonists, and do not induce substantial amounts of interferon alpha, may be desirable for use with certain vaccines such as bacterial vaccines because TLR7 induces type I IFN production, which down-regulates the production of IL-12 from macrophages and DCs. IL-12 contributes to the subsequent activation of macrophages, NK cells and CTLs, all of which contribute to anti-bacterial immunity. Therefore the induction of anti-bacterial immunity against some kinds of bacteria may be enhanced in the absence of IFNa.

For purposes of the present application, one way to determine if an IRM compound is considered to be an agonist for a particular TLR is if it activates an NFkB/luciferase reporter construct through that TLR from the target species more than about 1.5 fold, and usually at least about 2 fold, in TLR transfected host cells such as, e.g., HEK293 or Namalwa cells relative to control transfectants. For information regarding TLR activation, see, e.g., International Patent Publication Nos. WO 03/043573 and WO 03/043588, U.S. Patent Publication Nos. US2004/0014779, US2004/0132079; 2004/0162309; US2004/0171086, and US2004/0197865; and the other IRM patents and applications disclosed herein.

Preferred IRM compounds include a 2-aminopyridine fused to a five-membered nitrogen-containing heterocyclic ring.

Examples of classes of small molecule IRM compounds include, but are not limited to, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines, such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; and U.S. Patent Publication Nos. 2004/0091491; 2004/0147543; and 2004/0176367.

Additional examples of small molecule IRMs said to induce interferon (among other things) include purine derivatives (such as those described in U.S. Pat. Nos. 6,376,501, and 6,028,076), imidazoquinoline amide derivatives (such as those described in U.S. Pat. No. 6,069,149), and benzimidazole derivatives (such as those described in U.S. Pat. No. 6,387,938). 1H-imidazopyridine derivatives (such as those described in U.S. Pat. No. 6,518,265) are said to inhibit TNF and IL-1 cytokines. Other small molecule IRMs said to be TLR 7 agonists are shown in U.S. 2003/0199461 A1.

Examples of small molecule IRMs that include a 4-aminopyrimidine fused to a five-membered nitrogen-containing heterocyclic ring include adenine derivatives (such as those described in U.S. Pat. Nos. 6,376,501; 6,028,076 and 6,329,381; and in WO 02/08595).

Exemplary IRM Compounds

As noted above, many of the IRM compounds useful in the present invention have demonstrated immunomodulating activity. In certain embodiments of the present invention the IRM compound can be chosen from 1H-imidazo[4,5-c]quinolin-4-amines defined by one of Formulas I-V below: 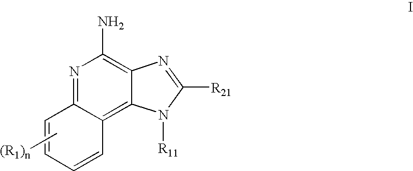
wherein

R₁₁ is selected from alkyl of one to ten carbon atoms, hydroxyalkyl of one to six carbon atoms, acyloxyalkyl wherein the acyloxy moiety is alkanoyloxy of two to four carbon atoms or benzoyloxy, and the alkyl moiety contains one to six carbon atoms, benzyl, (phenyl)ethyl and phenyl, said benzyl, (phenyl)ethyl or phenyl substituent being optionally substituted on the benzene ring by one or two moieties independently selected from alkyl of one to four carbon atoms, alkoxy of one to four carbon atoms and halogen, with the proviso that if said benzene ring is substituted by two of said moieties, then said moieties together contain no more than six carbon atoms;

R₂₁ is selected from hydrogen, alkyl of one to eight carbon atoms, benzyl, (phenyl)ethyl and phenyl, the benzyl, (phenyl)ethyl or phenyl substituent being optionally substituted on the benzene ring by one or two moieties independently selected from alkyl of one to four carbon atoms, alkoxy of one to four carbon atoms and halogen, with the proviso that when the benzene ring is substituted by two of said moieties, then the moieties together contain no more than six carbon atoms; and

each R₁ is independently selected from alkoxy of one to four carbon atoms, halogen, and alkyl of one to four carbon atoms, and n is an integer from 0 to 2, with the proviso that if n is 2, then said R₁ groups together contain no more than six carbon atoms; 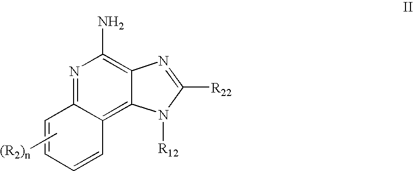
wherein

R₁₂ is selected from straight chain or branched chain alkenyl containing two to ten carbon atoms and substituted straight chain or branched chain alkenyl containing two to ten carbon atoms, wherein the substituent is selected from straight chain or branched chain alkyl containing one to four carbon atoms and cycloalkyl containing three to six carbon atoms; and cycloalkyl containing three to six carbon atoms substituted by straight chain or branched chain alkyl containing one to four carbon atoms; and

R₂₂ is selected from hydrogen, straight chain or branched chain alkyl containing one to eight carbon atoms, benzyl, (phenyl)ethyl and phenyl, the benzyl, (phenyl)ethyl or phenyl substituent being optionally substituted on the benzene ring by one or two moieties independently selected from straight chain or branched chain alkyl containing one to four carbon atoms, straight chain or branched chain alkoxy containing one to four carbon atoms, and halogen, with the proviso that when the benzene ring is substituted by two such moieties, then the moieties together contain no more than six carbon atoms; and

each R₂ is independently selected from straight chain or branched chain alkoxy containing one to four carbon atoms, halogen, and straight chain or branched chain alkyl containing one to four carbon atoms, and n is an integer from zero to 2, with the proviso that if n is 2, then said R₂ groups together contain no more than six carbon atoms; 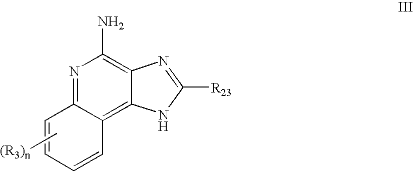
wherein

R₂₃ is selected from hydrogen, straight chain or branched chain alkyl of one to eight carbon atoms, benzyl, (phenyl)ethyl and phenyl, the benzyl, (phenyl)ethyl or phenyl substituent being optionally substituted on the benzene ring by one or two moieties independently selected from straight chain or branched chain alkyl of one to four carbon atoms, straight chain or branched chain alkoxy of one to four carbon atoms, and halogen, with the proviso that when the benzene ring is substituted by two such moieties, then the moieties together contain no more than six carbon atoms; and

each R₃ is independently selected from straight chain or branched chain alkoxy of one to four carbon atoms, halogen, and straight chain or branched chain alkyl of one to four carbon atoms, and n is an integer from zero to 2, with the proviso that if n is 2, then said R₃ groups together contain no more than six carbon atoms; 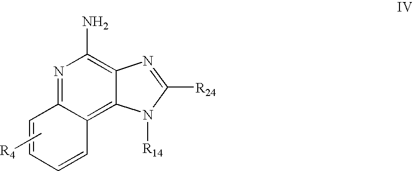
wherein

R₁₄ is —CHRₓR_{y} wherein R_{y} is hydrogen or a carbon-carbon bond, with the proviso that when R_{y} is hydrogen Rₓ is alkoxy of one to four carbon atoms, hydroxyalkoxy of one to four carbon atoms, 1-alkynyl of two to ten carbon atoms, tetrahydropyranyl, alkoxyalkyl wherein the alkoxy moiety contains one to four carbon atoms and the alkyl moiety contains one to four carbon atoms, or 2-, 3-, or 4-pyridyl, and with the further proviso that when R_{y} is a carbon-carbon bond R_{y} and Rₓ together form a tetrahydrofuranyl group optionally substituted with one or more substituents independently selected from hydroxy and hydroxyalkyl of one to four carbon atoms;

R₂₄ is selected from hydrogen, alkyl of one to four carbon atoms, phenyl, and substituted phenyl wherein the substituent is selected from alkyl of one to four carbon atoms, alkoxy of one to four carbon atoms, and halogen; and

R₄ is selected from hydrogen, straight chain or branched chain alkoxy containing one to four carbon atoms, halogen, and straight chain or branched chain alkyl containing one to four carbon atoms; 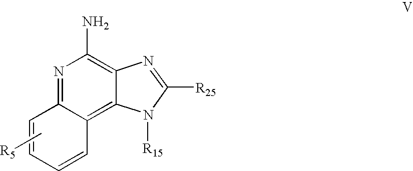
wherein

R₁₅ is selected from hydrogen; straight chain or branched chain alkyl containing one to ten carbon atoms and substituted straight chain or branched chain alkyl containing one to ten carbon atoms, wherein the substituent is selected from cycloalkyl containing three to six carbon atoms and cycloalkyl containing three to six carbon atoms substituted by straight chain or branched chain alkyl containing one to four carbon atoms; straight chain or branched chain alkenyl containing two to ten carbon atoms and substituted straight chain or branched chain alkenyl containing two to ten carbon atoms, wherein the substituent is selected from cycloalkyl containing three to six carbon atoms and cycloalkyl containing three to six carbon atoms substituted by straight chain or branched chain alkyl containing one to four carbon atoms; hydroxyalkyl of one to six carbon atoms; alkoxyalkyl wherein the alkoxy moiety contains one to four carbon atoms and the alkyl moiety contains one to six carbon atoms; acyloxyalkyl wherein the acyloxy moiety is alkanoyloxy of two to four carbon atoms or benzoyloxy, and the alkyl moiety contains one to six carbon atoms; benzyl; (phenyl)ethyl; and phenyl; said benzyl, (phenyl)ethyl or phenyl substituent being optionally substituted on the benzene ring by one or two moieties independently selected from alkyl of one to four carbon atoms, alkoxy of one to four carbon atoms, and halogen, with the proviso that when said benzene ring is substituted by two of said moieties, then the moieties together contain no more than six carbon atoms;

R₂₅ is 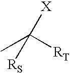
wherein

R_{S} and R_{T} are independently selected from hydrogen, alkyl of one to four carbon atoms, phenyl, and substituted phenyl wherein the substituent is selected from alkyl of one to four carbon atoms, alkoxy of one to four carbon atoms, and halogen;

X is selected from alkoxy containing one to four carbon atoms, alkoxyalkyl wherein the alkoxy moiety contains one to four carbon atoms and the alkyl moiety contains one to four carbon atoms, hydroxyalkyl of one to four carbon atoms, haloalkyl of one to four carbon atoms, alkylamido wherein the alkyl group contains one to four carbon atoms, amino, substituted amino wherein the substituent is alkyl or hydroxyalkyl of one to four carbon atoms, azido, chloro, hydroxy, 1-morpholino, 1-pyrrolidino, alkylthio of one to four carbon atoms; and

R₅ is selected from hydrogen, straight chain or branched chain alkoxy containing one to four carbon atoms, halogen, and straight chain or branched chain alkyl containing one to four carbon atoms;

and pharmaceutically acceptable salts of any of the foregoing.

In another embodiment, the IRM compound can be chosen from 6,7 fused cycloalkylimidazopyridine amines defined by Formula VI below: 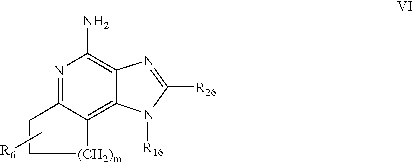
wherein

m is 1, 2, or 3;

R₁₆ is selected from hydrogen; cyclic alkyl of three, four, or five carbon atoms; straight chain or branched chain alkyl containing one to ten carbon atoms and substituted straight chain or branched chain alkyl containing one to ten carbon atoms, wherein the substituent is selected from cycloalkyl containing three to six carbon atoms and cycloalkyl containing three to six carbon atoms substituted by straight chain or branched chain alkyl containing one to four carbon atoms; fluoro- or chloroalkyl containing from one to ten carbon atoms and one or more fluorine or chlorine atoms; straight chain or branched chain alkenyl containing two to ten carbon atoms and substituted straight chain or branched chain alkenyl containing two to ten carbon atoms, wherein the substituent is selected from cycloalkyl containing three to six carbon atoms and cycloalkyl containing three to six carbon atoms substituted by straight chain or branched chain alkyl containing one to four carbon atoms; hydroxyalkyl of one to six carbon atoms; alkoxyalkyl wherein the alkoxy moiety contains one to four carbon atoms and the alkyl moiety contains one to six carbon atoms; acyloxyalkyl wherein the acyloxy moiety is alkanoyloxy of two to four carbon atoms or benzoyloxy, and the alkyl moiety contains one to six carbon atoms, with the proviso that any such alkyl, substituted alkyl, alkenyl, substituted alkenyl, hydroxyalkyl, alkoxyalkyl, or acyloxyalkyl group does not have a fully carbon substituted carbon atom bonded directly to the nitrogen atom; benzyl; (phenyl)ethyl; and phenyl; said benzyl, (phenyl)ethyl or phenyl substituent being optionally substituted on the benzene ring by one or two moieties independently selected from alkyl of one to four carbon atoms, alkoxy of one to four carbon atoms, and halogen, with the proviso that when said benzene ring is substituted by two of said moieties, then the moieties together contain no more than six carbon atoms; and —CHRₓR_{y}

wherein

R_{y} is hydrogen or a carbon-carbon bond, with the proviso that when R_{y} is hydrogen Rₓ is alkoxy of one to four carbon atoms, hydroxyalkoxy of one to four carbon atoms, 1-alkynyl of two to ten carbon atoms, tetrahydropyranyl, alkoxyalkyl wherein the alkoxy moiety contains one to four carbon atoms and the alkyl moiety contains one to four carbon atoms, 2-, 3-, or 4-pyridyl, and with the further proviso that when R_{y} is a carbon-carbon bond R_{y} and Rₓ together form a tetrahydrofuranyl group optionally substituted with one or more substituents independently selected from hydroxy and hydroxyalkyl of one to four carbon atoms,

R₂₆ is selected from hydrogen; straight chain or branched chain alkyl containing one to eight carbon atoms; straight chain or branched chain hydroxyalkyl containing one to six carbon atoms; morpholinoalkyl; benzyl; (phenyl)ethyl; and phenyl, the benzyl, (phenyl)ethyl, or phenyl substituent being optionally substituted on the benzene ring by a moiety selected from methyl, methoxy, and halogen; and —C(R_{S})(R_{T})(X) wherein R_{S} and R_{T} are independently selected from hydrogen, alkyl of one to four carbon atoms, phenyl, and substituted phenyl wherein the substituent is selected from alkyl of one to four carbon atoms, alkoxy of one to four carbon atoms, and halogen;

X is selected from alkoxy containing one to four carbon atoms, alkoxyalkyl wherein the alkoxy moiety contains one to four carbon atoms and the alkyl moiety contains one to four carbon atoms, haloalkyl of one to four carbon atoms, alkylamido wherein the alkyl group contains one to four carbon atoms, amino, substituted amino wherein the substituent is alkyl or hydroxyalkyl of one to four carbon atoms, azido, alkylthio of one to four carbon atoms, and morpholinoalkyl wherein the alkyl moiety contains one to four carbon atoms, and

R₆ is selected from hydrogen, fluoro, chloro, straight chain or branched chain alkyl containing one to four carbon atoms, and straight chain or branched chain fluoro- or chloroalkyl containing one to four carbon atoms and at least one fluorine or chlorine atom; and pharmaceutically acceptable salts thereof.

In another embodiment, the IRM compound can be chosen from imidazopyridine amines defined by Formula VII below: 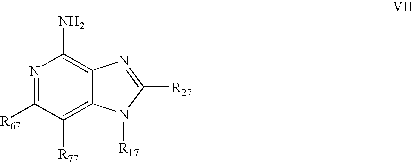
wherein

R₁₇ is selected from hydrogen; —CH₂R_{W} wherein R_{W} is selected from straight chain, branched chain, or cyclic alkyl containing one to ten carbon atoms, straight chain or branched chain alkenyl containing two to ten carbon atoms, straight chain or branched chain hydroxyalkyl containing one to six carbon atoms, alkoxyalkyl wherein the alkoxy moiety contains one to four carbon atoms and the alkyl moiety contains one to six carbon atoms, and phenylethyl; and —CH═CR_{Z}R_{Z} wherein each R_{Z} is independently straight chain, branched chain, or cyclic alkyl of one to six carbon atoms;

R₂₇ is selected from hydrogen; straight chain or branched chain alkyl containing one to eight carbon atoms; straight chain or branched chain hydroxyalkyl containing one to six carbon atoms; alkoxyalkyl wherein the alkoxy moiety contains one to four carbon atoms and the alkyl moiety contains one to six carbon atoms; benzyl, (phenyl)ethyl and phenyl, the benzyl, (phenyl)ethyl and phenyl being optionally substituted on the benzene ring by a moiety selected from methyl, methoxy, and halogen; and morpholinoalkyl wherein the alkyl moiety contains one to four carbon atoms;

R₆₇ and R₇₇ are independently selected from hydrogen and alkyl of one to five carbon atoms, with the proviso that R₆₇ and R₇₇ taken together contain no more than six carbon atoms, and with the further proviso that when R₇₇ is hydrogen then R₆₇ is other than hydrogen and R₂₇ is other than hydrogen or morpholinoalkyl, and with the further proviso that when R₆₇ is hydrogen then R₇₇ and R₂₇ are other than hydrogen; and pharmaceutically acceptable salts thereof.

In another embodiment, the IRM compound can be chosen from 1,2-bridged imidazoquinoline amines defined by Formula VIII below: 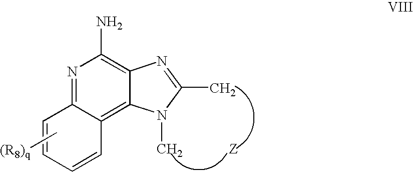
wherein

Z is selected from

—(CH₂)ₚ— wherein p is 1 to 4;

—(CH₂)ₐ—C(R_{D}R_{E})(CH₂)_{b}—, wherein a and b are integers and a+b is 0 to 3, R_{D} is hydrogen or alkyl of one to four carbon atoms, and R_{E} is selected from alkyl of one to four carbon atoms, hydroxy, —OR_{F} wherein R_{F} is alkyl of one to four carbon atoms, and —NR_{G}R′_{G} wherein R_{G} and R′_{G} are independently hydrogen or alkyl of one to four carbon atoms; and

—(CH₂)a—(Y)—(CH₂)_{b}— wherein a and b are integers and a+b is 0 to 3, and Y is O, S, or —NR_{J}— wherein R_{J} is hydrogen or alkyl of one to four carbon atoms;

q is 0 or 1; and

R₈ is selected from alkyl of one to four carbon atoms, alkoxy of one to four carbon atoms, and halogen,

and pharmaceutically acceptable salts thereof.

In another embodiment, the IRM compound can be chosen from thiazoloquinoline amines, oxazoloquinoline amines, thiazolopyridine amines, oxazolopyridine amines, thiazolonaphthyridine amines and oxazolonaphthyridine amines defined by Formula IX below: 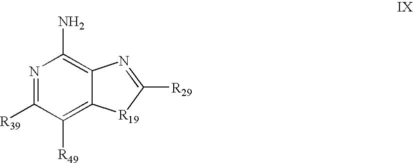
wherein:

R₁₉ is selected from oxygen, sulfur and selenium;

R₂₉ is selected from
-hydrogen;
-alkyl;
-alkyl-OH;
-haloalkyl;
-alkenyl;
-alkyl-X-alkyl;
-alkyl-X-alkenyl;
-alkenyl-X-alkyl;
-alkenyl-X-alkenyl;
-alkyl-N(R₅₉)₂;
-alkyl-N₃;
-alkyl-O—C(O)—N(R₅₉)₂;
-heterocyclyl;
-alkyl-X-heterocyclyl;
-alkenyl-X-heterocyclyl;
-aryl;
-alkyl-X-aryl;
-alkenyl-X-aryl;
-heteroaryl;
-alkyl-X-heteroaryl; and
-alkenyl-X-heteroaryl;

R₃₉ and R₄₉ are each independently:
-hydrogen;
—X-alkyl;
-halo;
-haloalkyl;
—N(R₅₉)₂;
or when taken together, R₃₉ and R₄₉ form a fused aromatic, heteroaromatic, cycloalkyl or heterocyclic ring;

X is selected from —O—, —S—, —NR₅₉—, —C(O)—, —C(O)O—, —OC(O)—, and a bond; and

each R₅₉ is independently H or C₁₋₈alkyl;

and pharmaceutically acceptable salts thereof.

In another embodiment, the IRM compound can be chosen from imidazonaphthyridine amines and imidazotetrahydronaphthyridine amines defined by Formulas X and XI below: 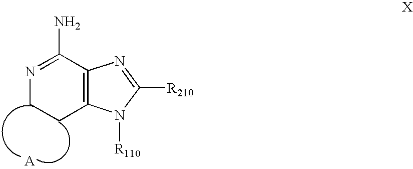
wherein

A is ═N—CR═CR—CR═; ═CR—N═CR—CR═; ═CR—CR═N—CR═; or ═CR—CR═CR—N═;

R₁₁₀ is selected from:

-hydrogen;

—C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀ alkyl;
—O—(C₁₋₂₀ alkyl)₀₋₁-aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂—(C₁₋₂₀ alkyl)₀₋₁-aryl;
—S(O)₀₋₂—(C₁₋₂₀ alkyl)₀₋₁-heteroaryl;
—S(O)₀₋₂—(C₁₋₂₀ alkyl)₀₋₁-heterocyclyl;
—N(R₃₁₀)₂;
—N₃;
oxo;
-halogen;
—NO₂;
—OH; and
—SH; and

—C₁₋₂₀ alkyl-NR₃₁₀-Q-X—R₄₁₀ or —C₂₋₂₀ alkenyl-NR₃₁₀-Q-X—R₄₁₀ wherein Q is —CO— or —SO₂—; X is a bond, —O— or —NR₃₁₀— and R₄₁₀ is aryl; heteroaryl; heterocyclyl; or —C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀ alkyl;
—O—(C₁₋₂₀ alkyl)₀₋₁-aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂—(C₁₋₂₀ alkyl)₀₋₁-aryl;
—S(O)₀₋₂—(C₁₋₂₀ alkyl)₀₋₁-heteroaryl;
—S(O)₀₋₂—(C₁₋₂₀ alkyl)₀₋₁-heterocyclyl;
—N(R₃₁₀)₂;
—NR₃₁₀—CO—O—C₁₋₂₀ alkyl;
—N₃;
oxo;
-halogen;
—NO₂;
—OH; and
—SH; or R₄₁₀ is 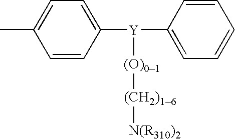
wherein Y is —N— or —CR—;
R₂₁₀ is selected from:
   -hydrogen;
   —C₁₋₁₀ alkyl;
   —C₂₋₁₀ alkenyl;
   -aryl;
   —C₁₋₁₀ alkyl-O—C₁₋₁₀ alkyl;
   —C₁₋₁₀ alkyl-O—C₂₋₁₀ alkenyl; and
   —C₁₋₁₀ alkyl or C₂₋₁₀ alkenyl substituted by one or more substituents selected from:
      —OH;
      -halogen;
      —N(R₃₁₀)₂;
      —CO—N(R₃₁₀)₂;
      —CO—C₁₋₁₀ alkyl;
      —N₃;
      -aryl;
      -heteroaryl;
      -heterocyclyl;
      —CO-aryl; and
      —CO-heteroaryl;

each R₃₁₀ is independently selected from hydrogen and C₁₋₁₀ alkyl; and

each R is independently selected from hydrogen, C₁₋₁₀ alkyl, C₁₋₁₀ alkoxy, halogen and trifluoromethyl; 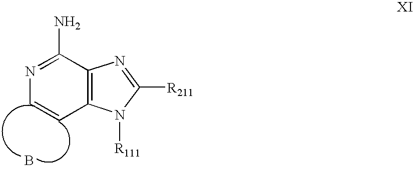
wherein

B is —NR—C(R)₂—C(R)₂—C(R)₂—; —C(R)₂—NR—C(R)₂—C(R)₂—; —C(R)₂—C(R)₂—NR—C(R)₂— or —C(R)₂—C(R)₂—C(R)₂—NR—;

R₁₁₁ is selected from:

-hydrogen;

—C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀ alkyl;
—O—(C₁₋₂₀ alkyl)₀₋₁-aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂—(C₁₋₂₀ alkyl)₀₋₁-aryl;
—S(O)₀₋₂—(C₁₋₂₀ alkyl)₀₋₁-heteroaryl;
—S(O)₀₋₂—(C₁₋₂₀ alkyl)₀₋₁-heterocyclyl;
—N(R₃₁₁)₂;
—N₃;
oxo;
-halogen;
—NO₂;
—OH; and
—SH; and

—C₁₋₂₀ alkyl-NR₃₁₁-Q-X—R₄₁₁ or —C₂₋₂₀ alkenyl-NR₃₁₁-Q-X—R₄₁₁ wherein Q is —CO— or —SO₂—; X is a bond, —O— or —NR₃₁₁— and R₄₁₁ is aryl; heteroaryl; heterocyclyl; or —C₁₋₂₀ alkyl or C₂₋₂₀ alkenyl that is unsubstituted or substituted by one or more substituents selected from:
-aryl;
-heteroaryl;
-heterocyclyl;
—O—C₁₋₂₀ alkyl;
—O—(C₁₋₂₀ alkyl)₀₋₁-aryl;
—O—(C₁₋₂₀ alkyl)₀₋₁-heteroaryl;
—O—(C₁₋₂₀ alkyl)₀₋₁-heterocyclyl;
—CO—O—C₁₋₂₀ alkyl;
—S(O)₀₋₂—C₁₋₂₀ alkyl;
—S(O)₀₋₂—(C₁₋₂₀ alkyl)₀₋₁-aryl;
—S(O)₀₋₂-(C₁₋₂₀ alkyl)₀₋₁-heteroaryl;
—S(O)₀₋₂-(C₁₋₂₀ alkyl)₀₋₁-heterocyclyl;
—N(R₃₁₁)₂;
—NR₃₁₁—CO—O—C₁₋₂₀ alkyl;
—N₃;
oxo;
-halogen;
—NO₂;
—OH; and
—SH; or R₄₁₁ is 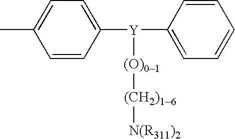
wherein Y is —N— or —CR—;
R₂₁₁ is selected from:
   -hydrogen;
   —C₁₋₁₀ alkyl;
   —C₂₋₁₀ alkenyl;
   -aryl;
   —C₁₋₁₀ alkyl-O—C₁₋₁₀-alkyl;
   —C₁₋₁₀ alkyl-O—C₂₋₁₀ alkenyl; and
   —C₁₋₁₀ alkyl or C₂₋₁₀ alkenyl substituted by one or more substituents selected from:
      —OH;
      -halogen;
      —N(R₃₁₁)₂;
      —CO—N(R₃₁₁)₂;
      —CO—C₁₋₁₀ alkyl;
      —N₃;
      -aryl;
      -heteroaryl;
      -heterocyclyl;
      —CO-aryl; and
      —CO-heteroaryl;

each R₃₁₁ is independently selected from hydrogen and C₁₋₁₀ alkyl; and

each R is independently selected from hydrogen, C₁₋₁₀ alkyl, C₁₋₁₀ alkoxy, halogen, and trifluoromethyl;

and pharmaceutically acceptable salts thereof.

In another embodiment, the IRM compound can be chosen from 1H-imidazo[4,5-c]quinolin-4-amines and tetrahydro-1H-imidazo[4,5-c]quinolin-4-amines defined by Formulas XII, XIII and XIV below: 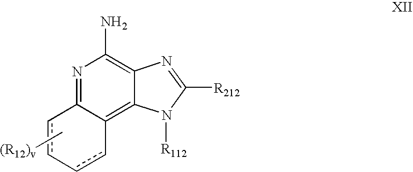
wherein

R₁₁₂ is -alkyl-NR₃₁₂—CO—R₄₁₂ or -alkenyl-NR₃₁₂—CO—R₄₁₂ wherein R₄₁₂ is aryl, heteroaryl, alkyl or alkenyl, each of which may be unsubstituted or substituted by one or more substituents selected from:
-alkyl;
-alkenyl;
-alkynyl;
-(alkyl)₀₋₁-aryl;
-(alkyl)₀₋₁-(substituted aryl);
-(alkyl)₀₋₁-heteroaryl;
-(alkyl)₀₋₁-(substituted heteroaryl);
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
—CO-aryl;
—CO-(substituted aryl);
—CO-heteroaryl;
—CO-(substituted heteroaryl);
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-(substituted aryl);
—S(O)₀₋₂-(alkyl)₀₋₁-heteroaryl;
—S(O)₀₋₂-(alkyl)₀₋₁-(substituted heteroaryl);
—P(O)(OR₃₁₂)₂;
—NR₃₁₂—CO—O-alkyl;
—N₃;
-halogen;
—NO₂;
—CN;
-haloalkyl;
—O-haloalkyl;
—CO-haloalkyl;
—OH;
—SH; and in the case that R₄₁₂ is alkyl, alkenyl, or heterocyclyl, oxo;
or R₄₁₂ is

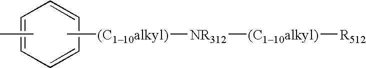

wherein R₅₁₂ is an aryl, (substituted aryl), heteroaryl, (substituted heteroaryl), heterocyclyl or (substituted heterocyclyl) group;

R₂₁₂ is selected from:
-hydrogen;
-alkyl;
-alkenyl;
-aryl;
-(substituted aryl);
-heteroaryl;
-(substituted heteroaryl);
-heterocyclyl;
-(substituted heterocyclyl);
-alkyl-O-alkyl;
-alkyl-O-alkenyl; and
-alkyl or alkenyl substituted by one or more substituents selected from:
   —OH;
   -halogen;
   —N(R₃₁₂)₂;
   —CO—N(R₃₁₂)₂;
   —CO—C₁₋₁₀ alkyl;
   —CO—O—C₁₋₁₀ alkyl;
   —N₃;
   -aryl;
   -(substituted aryl);
   -heteroaryl;
   -(substituted heteroaryl);
   -heterocyclyl;
   -(substituted heterocyclyl);
   —CO-aryl; and
   —CO-heteroaryl;

each R₃₁₂ is independently selected from hydrogen; C₁₋₁₀ alkyl-heteroaryl; C₁₋₁₀ alkyl-(substituted heteroaryl); C₁₋₁₀ alkyl-aryl; C₁₋₁₀ alkyl-(substituted aryl) and C₁₋₁₀ alkyl;

v is 0 to 4;

and each R₁₂ present is independently selected from C₁₋₁₀ alkyl, C₁₋₁₀ alkoxy, halogen, and trifluoromethyl; 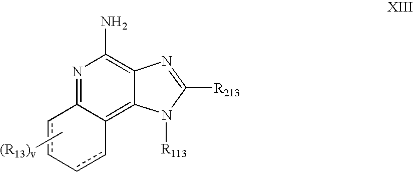
wherein

R₁₁₃ is -alkyl-NR₃₁₃—SO₂—X—R₄₁₃ or -alkenyl-NR₃₁₃—SO₂—X—R₄₁₃;

X is a bond or —NR₅₁₃—;

R₄₁₃ is aryl, heteroaryl, heterocyclyl, alkyl or alkenyl, each of which may be unsubstituted or substituted by one or more substituents selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁-aryl;
—O-(alkyl)₀₋₁-substituted aryl;
—O-(alkyl)₀₋₁-heteroaryl;
—O-(alkyl)₀₋₁substituted heteroaryl;
—O-(alkyl)₀₋₁-heterocyclyl;
—O-(alkyl)₀₋₁-substituted heterocyclyl;
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-substituted aryl;
—S(O)₀₋₂-(alkyl)₀₋₁-heteroaryl;
—S(O)₀₋₂-(alkyl)₀₋₁-substituted heteroaryl;
—S(O)₀₋₂-(alkyl)₀₋₁-heterocyclyl;
—S(O)₀₋₂-(alkyl)₀₋₁-substituted heterocyclyl;
-(alkyl)₀₋₁-NR₃₁₃R₃₁₃;
-(alkyl)₀₋₁-NR₃₁₃—CO—O-alkyl;
-(alkyl)₀₋₁-NR₃₁₃—CO-alkyl;
-(alkyl)₀₋₁-NR₃₁₃—CO-aryl;
-(alkyl)₀₋₁-NR₃₁₃—CO-substituted aryl;
-(alkyl)₀₋₁-NR₃₁₃—CO-heteroaryl;
-(alkyl)₀₋₁-NR₃₁₃—CO-substituted heteroaryl;
—N₃;
-halogen;
-haloalkyl;
-haloalkoxy;
—CO-haloalkyl;
—CO-haloalkoxy;
—NO₂;
—CN;
—OH;
—SH; and in the case that R₄₁₃ is alkyl, alkenyl, or heterocyclyl, oxo;

R₂₁₃ is selected from:
-hydrogen;
-alkyl;
-alkenyl;
-aryl;
-substituted aryl;
-heteroaryl;
-substituted heteroaryl;
-alkyl-O-alkyl;
-alkyl-O-alkenyl; and
-alkyl or alkenyl substituted by one or more substituents selected from:
   —OH;
   -halogen;
   —N(R₃₁₃)₂;
   —CO—N(R₃₁₃)₂;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

each R₃₁₃ is independently selected from hydrogen and C₁₋₁₀ alkyl; or when X is a bond R₃₁₃ and R₄₁₃ can join to form a 3 to 7 membered heterocyclic or substituted heterocyclic ring;

R₅₁₃ is selected from hydrogen and C₁₋₁₀ alkyl, or R₄₁₃ and R₅₁₃ can combine to form a 3 to 7 membered heterocyclic or substituted heterocyclic ring;

v is 0 to 4;

and each R₁₃ present is independently selected from C₁₋₁₀ alkyl, C₁₋₁₀ alkoxy, halogen, and trifluoromethyl; 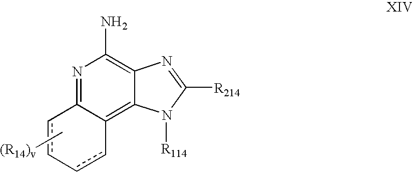
wherein

R₁₁₄ is -alkyl-NR₃₁₄—CY—NR₅₁₄—X—R₄₁₄ or

-alkenyl-NR₃₁₄—CY—NR₅₁₄—X—R₄₁₄

wherein

Y is ═O or ═S;

X is a bond, —CO— or —SO₂—;

R₄₁₄ is aryl, heteroaryl, heterocyclyl, alkyl or alkenyl, each of which may be unsubstituted or substituted by one or more substituents selected from:
-alkyl;
-alkenyl;
-aryl;
-heteroaryl;
-heterocyclyl;
-substituted aryl;
-substituted heteroaryl;
-substituted heterocyclyl;
—O-alkyl;
—O-(alkyl)₀₋₁-aryl;
—O-(alkyl)₀₋₁-substituted aryl;
—O-(alkyl)₀₋₁-heteroaryl;
—O-(alkyl)₀₋₁-substituted heteroaryl;
—O-(alkyl)₀₋₁-heterocyclyl;
—O-(alkyl)₀₋₁-substituted heterocyclyl;
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-substituted aryl;
—S(O)₀₋₂-(alkyl)₀₋₁-heteroaryl;
—S(O)₀₋₂-(alkyl)₀₋₁-substituted heteroaryl;
—S(O)₀₋₂-(alkyl)₀₋₁-heterocyclyl;
—S(O)₀₋₂-(alkyl)₀₋₁-substituted heterocyclyl;
-(alkyl)₀₋₁-NR₃₁₄R₃₁₄;
-(alkyl)₀₋₁-NR₃₁₄—CO—O-alkyl;
-(alkyl)₀₋₁-NR₃₁₄—CO-alkyl;
-(alkyl)₀₋₁-NR₃₁₄—CO-aryl;
-(alkyl)₀₋₁-NR₃₁₄—CO-substituted aryl;
-(alkyl)₀₋₁-NR₃₁₄—CO-heteroaryl;
-(alkyl)₀₋₁-NR₃₁₄—CO-substituted heteroaryl;
—N₃;
-halogen;
-haloalkyl;
-haloalkoxy;
—CO-haloalkoxy;
—NO₂;
—CN;
—OH;
—SH; and, in the case that R₄₁₄ is alkyl, alkenyl or heterocyclyl, oxo;

with the proviso that when X is a bond R₄₁₄ can additionally be hydrogen;

R₂₁₄ is selected from:
-hydrogen;
-alkyl;
-alkenyl;
-aryl;
-substituted aryl;
-heteroaryl;
-substituted heteroaryl;
-alkyl-O-alkyl;
-alkyl-O-alkenyl; and
-alkyl or alkenyl substituted by one or more substituents selected from:
   —OH;
   -halogen;
   —N(R₃₁₄)₂;
   —CO—N(R₃₁₄)₂;
   —CO—C₁₋₁₀ alkyl;
   —CO—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —CO-aryl;
   —CO-(substituted aryl);
   —CO-heteroaryl; and
   —CO-(substituted heteroaryl);

each R₃₁₄ is independently selected from hydrogen and C₁₋₁₀ alkyl;

R₅₁₄ is selected from hydrogen and C₁₋₁₀ alkyl, or R₄₁₄ and R₅₁₄ can combine to form a 3 to 7 membered heterocyclic or substituted heterocyclic ring;

v is 0 to 4;

and each R₁₄ present is independently selected from C₁₋₁₀ alkyl, C₁₋₁₀ alkoxy, halogen, and trifluoromethyl;

and pharmaceutically acceptable salts thereof.

In another embodiment, the IRM compound can be chosen from 1H-imidazo[4,5-c]quinolin-4-amines and tetrahydro-1H-imidazo[4,5-c]quinolin-4-amines defined by Formulas XV, XVI, XVII, XVIII, XIX, XX, XXI, XXII, XXIII, XXIV, XXV, and XXVI below: 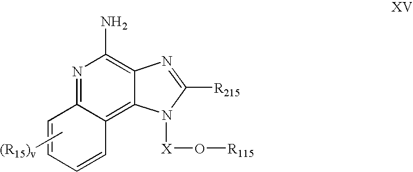
wherein: X is —CHR₅₁₅—, —CHR₅₁₅-alkyl-, or —CHR₅₁₅-alkenyl-;
R₁₁₅ is selected from:
   —R₄₁₅—CR₃₁₅-Z-R₆₁₅-alkyl;
   —R₄₁₅—CR₃₁₅-Z-R₆₁₅-alkenyl;
   —R₄₁₅—CR₃₁₅-Z-R₆₁₅-aryl;
   —R₄₁₅—CR₃₁₅-Z-R₆₁₅-heteroaryl;
   —R₄₁₅—CR₃₁₅-Z-R₆₁₅-heterocyclyl;
   —R₄₁₅—CR₃₁₅-Z-H;
   —R₄₁₅—NR₇₁₅—CR₃₁₅—R₆₁₅-alkyl;
   —R₄₁₅—NR₇₁₅—CR₃₁₅—R₆₁₅-alkenyl;
   —R₄₁₅—NR₇₁₅—CR₃₁₅—R₆₁₅-aryl;
   —R₄₁₅—NR₇₁₅—CR₃₁₅—R₆₁₅-heteroaryl;
   —R₄₁₅—NR₇₁₅—CR₃₁₅—R₆₁₅-heterocyclyl; and
   —R₄₁₅—NR₇₁₅—CR₃₁₅—R₈₁₅;
Z is —NR₅₁₅—, —O—, or —S—;
R₂₁₅ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₅₁₅)₂;
      —CO—N(R₅₁₅)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
R₃₁₅ is ═O or ═S;
R₄₁₅ is alkyl or alkenyl, which may be interrupted by one or more —O— groups;
each R₅₁₅ is independently H or C₁₋₁₀ alkyl;
R₆₁₅ is a bond, alkyl, or alkenyl, which may be interrupted by one or more —O— groups;
R₇₁₅ is H, C₁₋₁₀ alkyl, or arylalkyl; or R₄₁₅ and R₇₁₅ can join together to form a ring;
R₈₁₅ is H or C₁₋₁₀ alkyl; or R₇₁₅ and R₈₁₅ can join together to form a ring;
Y is —O— or —S(O)₀₋₂—;
v is 0 to 4; and
each R₁₅ present is independently selected from C₁₋₁₀ alkyl C₁₋₁₀ alkoxy, hydroxy, halogen, and trifluoromethyl; 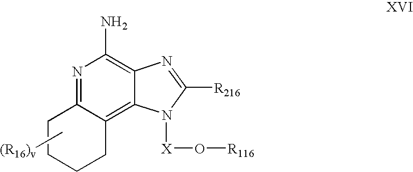
   wherein: X is —CHR₅₁₆—, —CHR₅₁₆-alkyl-, or —CHR₅₁₆-alkenyl-;
R₁₁₆ is selected from:
   —R₄₁₆—CR₃₁₆-Z-R₆₁₆-alkyl;
   —R₄₁₆—CR₃₁₆-Z-R₆₁₆-alkenyl;
   —R₄₁₆—CR₃₁₆-Z-R₆₁₆-aryl;
   —R₄₁₆—CR₃₁₆-Z-R₆₁₆-heteroaryl;
   —R₄₁₆—CR₃₁₆-Z-R₆₁₆-heterocyclyl;
   —R₄₁₆—CR₃₁₆-Z-H;
   —R₄₁₆—NR₇₁₆—CR₃₁₆—R₆₁₆-alkyl;
   —R₄₁₆—NR₇₁₆—CR₃₁₆—R₆₁₆-alkenyl;
   —R₄₁₆—NR₇₁₆—CR₃₁₆—R₆₁₆-aryl;
   —R₄₁₆—NR₇₁₆—CR₃₁₆—R₆₁₆-heteroaryl;
   —R₄₁₆—NR₇₁₆—CR₃₁₆—R₆₁₆-heterocyclyl; and
   —R₄₁₆—NR₇₁₆—CR₃₁₆—R₈₁₆;
Z is —NR₅₁₆—, —O—, or —S—;
R₂₁₆ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₅₁₆)₂;
      —CO—N(R₅₁₆)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
R₃₁₆ is ═O or ═S;
R₄₁₆ is alkyl or alkenyl, which may be interrupted by one or more —O— groups;
each R₅₁₆ is independently H or C₁₋₁₀ alkyl;
R₆₁₆ is a bond, alkyl, or alkenyl, which may be interrupted by one or more —O— groups;
R₇₁₆ is H, C₁₋₁₀ alkyl, arylalkyl; or R₄₁₆ and R₇₁₆ can join together to form a ring;
R₈₁₆ is H or C₁₋₁₀ alkyl; or R₇₁₆ and R₈₁₆ can join together to form a ring;
Y is —O— or —S(O)₀₋₂—;
v is 0 to 4; and
each R₁₆ present is independently selected from C₁₋₁₀ alkyl, C₁₋₁₀ alkoxy, hydroxy, halogen, and trifluoromethyl; 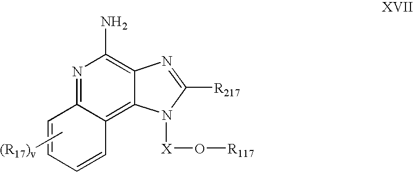
   wherein: X is —CHR₃₁₇—, —CHR₃₁₇-alkyl-, or —CHR₃₁₇-alkenyl-;
R₁₁₇ is selected from:
   -alkenyl;
   -aryl; and
   —R₄₁₇-aryl;
R₂₁₇ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   alkyl or alkenyl substituted by one or more substituents selected from:
      —OH;
      -halogen;
      —N(R₃₁₇)₂;
      —CO—N(R₃₁₇)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
R₄₁₇ is alkyl or alkenyl, which may be interrupted by one or more —O— groups;
each R₃₁₇ is independently H or C₁₋₁₀ alkyl;
each Y is independently —O— or —S(O)₀₋₂—;
v is 0 to 4; and
each R₁₇ present is independently selected from C₁₋₁₀ alkyl, C₁₋₁₀ alkoxy, hydroxy, halogen, and trifluoromethyl; 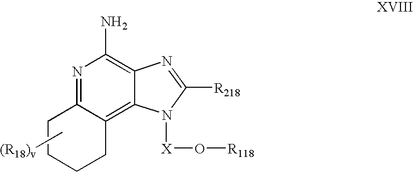
   wherein: X is —CHR₃₁₈—, —CHR₃₁₈-alkyl-, or —CHR₃₁₈-alkenyl-;
R₁₁₈ is selected from:
   -aryl;
   -alkenyl; and
   —R₄₁₈-aryl;
—R₂₁₈ is selected from:
   -hydrogen;
   -alkyl;
   -alkenyl;
   -aryl;
   -heteroaryl;
   -heterocyclyl;
   -alkyl-Y-alkyl;
   -alkyl-Y-aryl;
   -alkyl-Y-alkenyl; and
   -alkyl or alkenyl substituted by one or more substituents selected from:
      —OH;
      -halogen;
      —N(R₃₁₈)₂;
      —CO—N(R₃₁₈)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
R₄₁₈ is alkyl or alkenyl, which may be interrupted by one or more —O— groups;
each R₃₁₈ is independently H or C₁₋₁₀ alkyl;
each Y is independently —O— or —S(O)₀₋₂—;
v is 0 to 4; and
each R₃₁₈ present is independently selected C₁₋₁₀ alkyl, C₁₋₁₀ alkoxy, hydroxy, halogen, and trifluoromethyl; 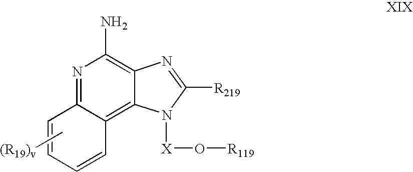
   wherein: X is —CHR₃₁₉—, —CHR₃₁₉-alkyl-, or —CHR₃₁₉-alkenyl-;
R₁₁₉ is selected from:
   -heteroaryl;
   -heterocyclyl;
   —R₄₁₉-heteroaryl; and
   —R₄₁₉-heterocyclyl;
R₂₁₉ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₃₁₉)₂;
      —CO—N(R₃₁₉)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
R₄₁₉ is alkyl or alkenyl, which may be interrupted by one or more —O— groups;
each R₃₁₉ is independently H or C₁₋₁₀ alkyl;
each Y is independently —O— or —S(O)₀₋₂—;
v is 0 to 4; and
each R₁₉ present is independently selected from C₁₋₁₀ alkyl, C₁₋₁₀ alkoxy, hydroxy, halogen, and trifluoromethyl; 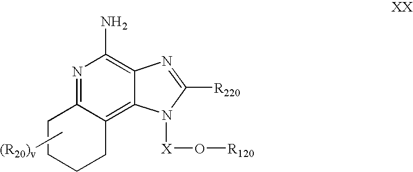
   wherein: X is —CHR₃₂₀—, —CHR₃₂₀-alkyl-, or —CHR₃₂₀-alkenyl-;
R₁₂₀ is selected from:
   -heteroaryl;
   -heterocyclyl;
   —R₄₂₀-heteroaryl; and
   —R₄₂₀-heterocyclyl;
R₂₂₀ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₃₂₀)₂;
      —CO—N(R₃₂₀)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
R₄₂₀ is alkyl or alkenyl, which may be interrupted by one or more —O— groups;
each R₃₂₀ is independently H or C₁₋₁₀ alkyl;
each Y is independently —O— or —S(O)₀₋₂—;
v is 0 to 4; and
each R₂₀ present is independently selected from C₁₋₁₀ alkyl, C₁₋₁₀ alkoxy, hydroxy, halogen, and trifluoromethyl; 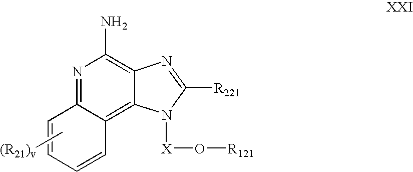
   wherein: X is —CHR₅₂₁—, —CHR₅₂₁-alkyl-, or —CHR₅₂₁-alkenyl-;
R₁₂₁ is selected from:
   —R₄₂₁—NR₃₂₁—SO₂—R₆₂₁-alkyl;
   —R₄₂₁—NR₃₂₁—SO₂—R₆₂₁-alkenyl;
   —R₄₂₁—NR₃₂₁—SO₂—R₆₂₁-aryl;
   —R₄₂₁—NR₃₂₁—SO₂—R₆₂₁-heteroaryl;
   —R₄₂₁—NR₃₂₁—SO₂—R₆₂₁-heterocyclyl;
   —R₄₂₁—NR₃₂₁—SO₂—R₇₂₁;
   —R₄₂₁—NR₃₂₁—SO₂—NR₅₂₁—R₆₂₁-alkyl;
   —R₄₂₁—NR₃₂₁—SO₂—NR₅₂₁—R₆₂₁-alkenyl;
   —R₄₂₁—NR₃₂₁—SO₂—NR₅₂₁—R₆₂₁-aryl;
   —R₄₂₁—NR₃₂₁—SO₂—NR₅₂₁—R₆₂₁-heteroaryl;
   —R₄₂₁—NR₃₂₁—SO₂—NR₅₂₁—R₆₂₁-heterocyclyl; and
   —R₄₂₁—NR₃₂₁—SO₂—NH₂;
R₂₂₁ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₅₂₁)₂;
      —CO—N(R₅₂₁)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
Y is —O— or —S(O)₀₋₂—;
R₃₂₁ is H, C₁₋₁₀ alkyl, or arylalkyl;
each R₄₂₁ is independently alkyl or alkenyl, which may be interrupted by one or more —O— groups; or R₃₂₁ and R₄₂₁ can join together to form a ring;
each R₅₂₁ is independently H, C₁₋₁₀ alkyl, or C₂₋₁₀ alkenyl;
R₆₂₁ is a bond, alkyl, or alkenyl, which may be interrupted by one or more —O— groups;
R₇₂₁ is C₁₋₁₀ alkyl; or R₃₂₁ and R₇₂₁ can join together to form a ring;

v is 0 to 4; and
each R₂₁ present is independently selected from C₁₋₁₀ alkyl, C₁₋₁₀ alkoxy, hydroxy, halogen, and trifluoromethyl; 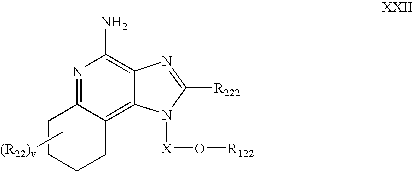
   wherein: X is —CHR₅₂₂—, —CHR₅₂₂-alkyl-, or —CHR₅₂₂-alkenyl-;
R₁₂₂ is selected from:
   —R₄₂₂—NR₃₂₂—SO₂—R₆₂₂-alkyl;
   —R₄₂₂—NR₃₂₂—SO₂—R₆₂₂-alkenyl;
   —R₄₂₂—NR₃₂₂—SO₂—R₆₂₂-aryl;
   —R₄₂₂—NR₃₂₂—SO₂—R₆₂₂-heteroaryl;
   —R₄₂₂—NR₃₂₂—SO₂—R₆₂₂-heterocyclyl;
   —R₄₂₂—NR₃₂₂—SO₂—R₇₂₂;
   —R₄₂₂—NR₃₂₂—SO₂—NR₅₂₂—R₆₂₂-alkyl;
   —R₄₂₂—NR₃₂₂—SO₂—NR₅₂₂—R₆₂₂-alkenyl;
   —R₄₂₂—NR₃₂₂—SO₂—NR₅₂₂—R₆₂₂-aryl;
   —R₄₂₂—NR₃₂₂—SO₂—NR₅₂₂—R₆₂₂-heteroaryl;
   —R₄₂₂—NR₃₂₂—SO₂—NR₅₂₂—R₆₂₂-heterocyclyl; and
   —R₄₂₂—NR₃₂₂—SO₂—NH₂;
R₂₂₂ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₅₂₂)₂;
      —CO—N(R₅₂₂)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
Y is —O— or —S(O)₀₋₂—;
R₃₂₂ is H, C₁₋₁₀ alkyl, or arylalkyl;
each R₄₂₂ is independently alkyl or alkenyl, which may be interrupted by one or more —O— groups; or R₃₂₂ and R₄₂₂ can join together to form a ring;
each R₅₂₂ is independently H, C₁₋₁₀ alkyl, or C₂₋₁₀ alkenyl;
R₆₂₂ is a bond, alkyl, or alkenyl, which may be interrupted by one or more —O— groups;
R₇₂₂ is C₁₋₁₀ alkyl; or R₃₂₂ and R₇₂₂ can join together to form a ring;
v is 0 to 4; and
each R₂₂ present is independently selected from C₁₋₁₀ alkyl, C₁₋₁₀ alkoxy, hydroxy, halogen, and trifluoromethyl; 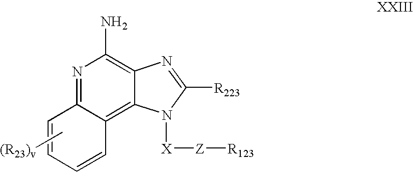
   wherein: X is —CHR₃₂₃—, —CHR₃₂₃-alkyl-, or —CHR₃₂₃-alkenyl-;
Z is —S—, —SO—, or —SO₂—;
R₁₂₃ is selected from:
   -alkyl;
   -aryl;
   -heteroaryl;
   -heterocyclyl;
   -alkenyl;
   —R₄₂₃-aryl;
   —R₄₂₃-heteroaryl; and
   —R₄₂₃-heterocyclyl;
R₂₂₃ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₃₂₃)₂;
      —CO—N(R₃₂₃)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
each R₃₂₃ is independently H or C₁₋₁₀ alkyl;
each R₄₂₃ is independently alkyl or alkenyl;
each Y is independently —O— or —S(O)₀₋₂—;
v is 0 to 4; and
each R₂₃ present is independently selected from C₁₋₁₀ alkyl, C₁₋₁₀ alkoxy, hydroxy, halogen, and trifluoromethyl; 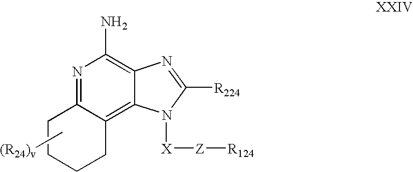
   wherein: X is —CHR₃₂₄—, —CHR₃₂₄-alkyl-, or —CHR₃₂₄-alkenyl-;
Z is —S—, —SO—, or —SO₂—;
R₁₂₄ is selected from:
   -alkyl;
   -aryl;
   -heteroaryl;
   -heterocyclyl;
   -alkenyl;
   —R₄₂₄-aryl;
   —R₄₂₄-heteroaryl; and
   —R₄₂₄-heterocyclyl;
R₂₂₄ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₃₂₄)₂;
      —CO—N(R₃₂₄)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
each R₃₂₄ is independently H or C₁₋₁₀ alkyl;
each R₄₂₄ is independently alkyl or alkenyl;
each Y is independently —O— or —S(O)₀₋₂—;
v is 0 to 4; and
each R₂₄ present is independently selected from C₁₋₁₀ alkyl, C₁₋₁₀ alkoxy, hydroxy, halogen, and trifluoromethyl; 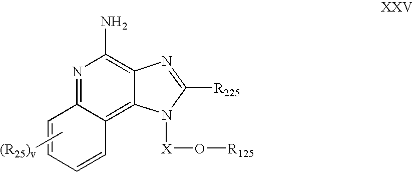
   wherein: X is —CHR₅₂₅—, —CHR₅₂₅-alkyl-, or —CHR₅₂₅-alkenyl-;
R₁₂₅ is selected from:
   —R₄₂₅—NR₈₂₅—CR₃₂₅—NR₅₂₅-Z-R₆₂₅-alkyl;
   —R₄₂₅—NR₈₂₅—CR₃₂₅—NR₅₂₅-Z-R₆₂₅-alkenyl;
   —R₄₂₅—NR₈₂₅—CR₃₂₅—NR₅₂₅-Z-R₆₂₅-aryl;
   —R₄₂₅—NR₈₂₅—CR₃₂₅—NR₅₂₅-Z-R₆₂₅-heteroaryl;
   —R₄₂₅—NR₈₂₅—CR₃₂₅—NR₅₂₅-Z-R₆₂₅-heterocyclyl;
   —R₄₂₅—NR₈₂₅—CR₃₂₅—NR₅₂₅R₇₂₅;
   —R₄₂₅—NR₈₂₅—CR₃₂₅—NR₉₂₅-Z-R₆₂₅-alkyl;
   —R₄₂₅—NR₈₂₅—CR₃₂₅—NR₉₂₅-Z-R₆₂₅-alkenyl;
   —R₄₂₅—NR₈₂₅—CR₃₂₅—NR₉₂₅-Z-R₆₂₅-aryl;
   —R₄₂₅—NR₈₂₅—CR₃₂₅—NR₉₂₅-Z-R₆₂₅-heteroaryl; and
   —R₄₂₅—NR₈₂₅—CR₃₂₅—NR₉₂₅-Z-R₆₂₅-heterocyclyl;
R₂₂₅ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₅₂₅)₂;
      —CO—N(R₅₂₅)₂;
      —CO—C₁₋₁₀ alkyl;
      —CO—O—C₁₋₁₀ alkyl;
      —N₃;
      -aryl;
      -heteroaryl;
      -heterocyclyl;
      —CO-aryl; and
      —CO-heteroaryl;
each R₃₂₅ is ═O or ═S;
each R₄₂₅ is independently alkyl or alkenyl, which may be interrupted by one or more —O— groups;
each R₅₂₅ is independently H or C₁₋₁₀ alkyl;
R₆₂₅ is a bond, alkyl, or alkenyl, which may be interrupted by one or more —O— groups;
R₇₂₅ is H or C₁₋₁₀ alkyl which may be interrupted by a hetero atom, or R₇₂₅ can join with R₅₂₅ to form a ring;
R₈₂₅ is H, C₁₋₁₀ alkyl, or arylalkyl; or R₄₂₅ and R₈₂₅ can join together to form a ring;
R₉₂₅ is C₁₋₁₀ alkyl which can join together with R₈₂₅ to form a ring;
each Y is independently —O— or —S(O)₀₋₂—;
Z is a bond, —CO—, or —SO₂—;
v is 0 to 4; and
each R₂₅ present is independently selected C₁₋₁₀ alkyl C₁₋₁₀ alkoxy, hydroxy, halogen, and trifluoromethyl; 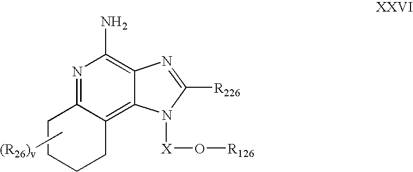
   wherein: X is —CHR₅₂₆—, —CHR₅₂₆-alkyl-, or —CHR₅₂₆-alkenyl-;
R₁₂₆ is selected from:
   —R₄₂₆—NR₈₂₆—CR₃₂₆—NR₅₂₆-Z-R₆₂₆-alkyl;
   —R₄₂₆—NR₈₂₆—CR₃₂₆—NR₅₂₆-Z-R₆₂₆-alkenyl;
   —R₄₂₆—NR₈₂₆—CR₃₂₆—NR₅₂₆-Z-R₆₂₆-aryl;
   —R₄₂₆—NR₈₂₆—CR₃₂₆—NR₅₂₆-Z-R₆₂₆-heteroaryl;
   —R₄₂₆—NR₈₂₆—CR₃₂₆—NR₅₂₆-Z-R₆₂₆-heterocyclyl;
   —R₄₂₆—NR₈₂₆—CR₃₂₆—NR₅₂₆R₇₂₆;
   —R₄₂₆—NR₈₂₆—CR₃₂₆—NR₉₂₆-Z-R₆₂₆-alkyl;
   —R₄₂₆—NR₈₂₆—CR₃₂₆—NR₉₂₆-Z-R₆₂₆-alkenyl;
   —R₄₂₆—NR₈₂₆—CR₃₂₆—NR₉₂₆-Z-R₆₂₆-aryl;
   —R₄₂₆—NR₈₂₆—CR₃₂₆—NR₉₂₆-Z-R₆₂₆-heteroaryl; and
   —R₄₂₆—NR₈₂₆—CR₃₂₆—NR₉₂₆-Z-R₆₂₆-heterocyclyl;
R₂₂₆ is selected from:
   -hydrogen;
   -alkyl;
   -alkenyl;
   -aryl;
   -heteroaryl;
   -heterocyclyl;
   -alkyl-Y-alkyl;
   -alkyl-Y-alkenyl;
   -alkyl-Y-aryl; and
   -alkyl or alkenyl substituted by one or more substituents selected from:
      —OH;
      -halogen;
      —N(R₅₂₆)₂;
      —CO—N(R₅₂₆)₂;
      —CO—C₁₋₁₀ alkyl;
      13 CO—O—C₁₋₁₀ alkyl;
      —N₃;
      -aryl;
      -heteroaryl;
      -heterocyclyl;
      —CO-aryl; and
      —CO-heteroaryl;
each R₃₂₆ is ═O or ═S;
each R₄₂₆ is independently alkyl or alkenyl, which may be interrupted by one or more —O— groups;
each R₅₂₆ is independently H or C₁₋₁₀ alkyl;
R₆₂₆ is a bond, alkyl, or alkenyl, which may be interrupted by one or more —O— groups;
R₇₂₆ is H or C₁₋₁₀ alkyl which may be interrupted by a hetero atom, or R₇₂₆ can join with R₅₂₆ to form a ring;
R₈₂₆ is H, C₁₋₁₀ alkyl, or arylalkyl; or R₄₂₆ and R₈₂₆ can join together to form a ring;
R₉₂₆ is C₁₋₁₀ alkyl which can join together with R₈₂₆ to form a ring;
each Y is independently —O— or —S(O)₀₋₂—;
Z is a bond, —CO—, or —SO₂—;
v is 0 to 4; and
each R₂₆ present is independently selected from C₁₋₁₀ alkyl, C₁₋₁₀ alkoxy, hydroxy, halogen, and trifluoromethyl;
   and pharmaceutically acceptable salts of any of the foregoing.

In another embodiment, the IRM compound can be chosen from 1H-imidazo[4,5-c]pyridin-4-amines defined by Formula XXVII below: 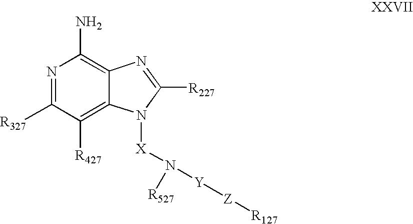
wherein X is alkylene or alkenylene;
Y is —CO— or —CS;
Z is a bond, —O—, or —S—;
R₁₂₇ is aryl, heteroaryl, heterocyclyl, alkyl or alkenyl, each of which may be unsubstituted or substituted by one or more substituents independently selected from:
-alkyl;
alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
—O-(alkyl)₀₋₁-heterocyclyl;
—O-(alkyl)₀₋₁-(substituted heterocyclyl);
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-(substituted aryl);
—S(O)₀₋₂-(alkyl)₀₋₁-heteroaryl;
—S(O)₀₋₂-(alkyl)₀₋₁-(substituted heteroaryl);
—S(O)₀₋₂-(alkyl)₀₋₁-heterocyclyl;
—S(O)₀₋₂-(alkyl)₀₋₁-(substituted heterocyclyl);
-(alkyl)₀₋₁-N(R₆₂₇)₂;
-(alkyl)₀₋₁-NR₆₂₇—CO—O-alkyl;
-(alkyl)₀₋₁-NR₆₂₇—CO-alkyl;
-(alkyl)₀₋₁-NR₆₂₇—CO-aryl;
-(alkyl)₀₋₁-NR₆₂₇—CO-(substituted aryl);
-(alkyl)₀₋₁-NR₆₂₇—CO-heteroaryl;
-(alkyl)₀₋₁-NR₆₂₇—CO-(substituted heteroaryl);
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂₂₇ is selected from:
   -hydrogen;
   -alkyl;
   -alkenyl;
   -aryl;
   -substituted aryl;
   -heteroaryl;
   -substituted heteroaryl;
   -alkyl-O-alkyl;
   -alkyl-S-alkyl;
   -alkyl-O-aryl;
   -alkyl-S-aryl:
   -alkyl-O-alkenyl;
   -alkyl-S-alkenyl; and
   -alkyl or alkenyl substituted by one or more substituents selected from:
      —OH;
      -halogen;
      —N(R₆₂₇)₂;
      —CO—N(R₆₂₇)₂;
      —CS—N(R₆₂₇)₂;
      —SO₂—N(R₆₂₇)₂;
      —NR₆₂₇—CO—C₁₋₁₀ alkyl;
      —NR₆₂₇—CS—C₁₋₁₀ alkyl;
      —NR₆₂₇—SO₂—C₁₋₁₀ alkyl;
      —CO—C₁₋₁₀ alkyl;
      —CO—O—C₁₋₁₀ alkyl;
      —N₃;
      -aryl;
      -substituted aryl;
      -heteroaryl;
      -substituted heteroaryl;
      -heterocyclyl;
      -substituted heterocyclyl;
      —CO-aryl;
      —CO-(substituted aryl);
      —CO-heteroaryl; and
      —CO-(substituted heteroaryl);
R₃₂₇ and R₄₂₇ are independently selected from hydrogen, alkyl, alkenyl, halogen, alkoxy, amino, alkylamino, dialkylamino, and alkylthio;
R₅₂₇ is H or C₁₋₁₀ alkyl, or R₅₂₇ can join with X to form a ring that contains one or two heteroatoms; or when R₁₂₇ is alkyl, R₅₂₇ and R₁₂₇ can join to form a ring;
each R₆₂₇ is independently H or C₁₋₁₀alkyl;
   and pharmaceutically acceptable salts thereof.

In another embodiment, the IRM compound can be chosen from 1H-imidazo[4,5-c]pyridin-4-amines defined by Formula XXVIII below: 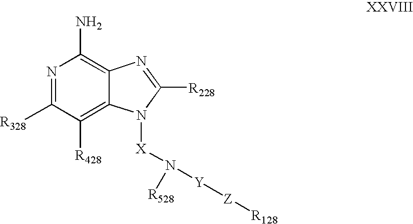
wherein X is alkylene or alkenylene;
Y is —SO₂—;
Z is a bond or —NR₆₂₈—;
R₁₂₈ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
—O-(alkyl)₀₋₁-heterocyclyl;
—O-(alkyl)₀₋₁-(substituted heterocyclyl);
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-(substituted aryl);
—S(O)₀₋₂-(alkyl)₀₋₁-heteroaryl;
—S(O)₀₋₂-(alkyl)₀₋₁-(substituted heteroaryl);
—S(O)₀₋₂-(alkyl)₀₋₁-heterocyclyl;
—S(O)₀₋₂-(alkyl)₀₋₁-(substituted heterocyclyl);
-(alkyl)₀₋₁-N(R₆₂₈)₂;
-(alkyl)₀₋₁-NR₆₂₈—CO—O-alkyl;
-(alkyl)₀₋₁-NR₆₂₈—CO-alkyl;
-(alkyl)₀₋₁-NR₆₂₈—CO-aryl;
-(alkyl)₀₋₁-NR₆₂₈—CO-(substituted aryl);
-(alkyl)₀₋₁-NR₆₂₈—CO-heteroaryl;
-(alkyl)₀₋₁-NR₆₂₈—CO-(substituted heteroaryl);
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂₂₈ is selected from:
   -hydrogen;
   -alkyl;
   -alkenyl;
   -aryl;
   -substituted aryl;
   -heteroaryl;
   -substituted heteroaryl;
   -alkyl-O-alkyl;
   -alkyl-S-alkyl;
   -alkyl-O-aryl;
   -alkyl-S-aryl:
   -alkyl-O-alkenyl;
   -alkyl-S-alkenyl; and
   -alkyl or alkenyl substituted by one or more substituents selected from:
      —OH;
      -halogen;
      —N(R₆₂₈)₂;
      —CO—N(R₆₂₈)₂;
      —CS—N(R₆₂₈)₂;
      —SO₂—N(R₆₂₈)₂;
      —NR₆₂₈—CO—C₁₋₁₀ alkyl;
      —N₆₂₈—CS—C₁₋₁₀ alkyl;
      —NR₆₂₈—SO₂—C₁₋₁₀ alkyl;
      —CO—C₁₋₁₀ alkyl;
      —CO—O—C₁₋₁₀ alkyl;
      —N₃;
      -aryl;
      -substituted aryl;
      -heteroaryl;
      -substituted heteroaryl;
      -heterocyclyl;
      -substituted heterocyclyl;
      —CO-aryl;
      —CO-(substituted aryl);
      —CO-heteroaryl; and
      —CO-(substituted heteroaryl);
R₃₂₈ and R₄₂₈ are independently selected from hydrogen, alkyl, alkenyl, halogen, alkoxy, amino, alkylamino, dialkylamino, and alkylthio;
R₅₂₈ is H or C₁₋₁₀ alkyl, or R₅₂₈ can join with X to form a ring; or when R₁₂₈ is alkyl, R₅₂₈ and R₁₂₈ can join to form a ring;
   each R₆₂₈ is independently H or C₁₋₁₀alkyl;

and pharmaceutically acceptable salts thereof.

In another embodiment, the IRM compound can be chosen from 1H-imidazo[4,5-c]pyridin-4-amines defined by Formula XXIX below: 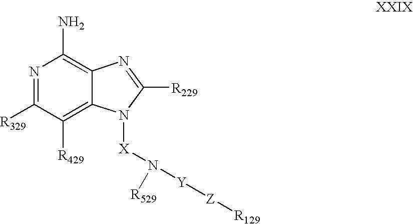
wherein X is alkylene or alkenylene;
Y is —CO— or —CS;
Z is —NR₆₂₉—, —NR₆₂₉—CO—, —NR₆₂₉—SO₂—, or —NR₇₂₉—;
R₁₂₉ is aryl, heteroaryl, heterocyclyl, alkyl or alkenyl, each of which may be unsubstituted or substituted by one or more substituents independently selected from:
-alkyl;
-alkenyl;
-aryl;
-heteroaryl;
-heterocyclyl;
-substituted cycloalkyl;
-substituted aryl;
-substituted heteroaryl;
-substituted heterocyclyl;
—O-alkyl;
—O-(alkyl)₀₋₁-aryl;
—O-(alkyl)₀₋₁-(substituted aryl);
—O-(alkyl)₀₋₁-heteroaryl;
—O-(alkyl)₀₋₁-(substituted heteroaryl);
—O-(alkyl)₀₋₁-heterocyclyl;
—O-(alkyl)₀₋₁-(substituted heterocyclyl);
—COOH;
—CO—O-alkyl;
—CO-alkyl;
—S(O)₀₋₂-alkyl;
—S(O)₀₋₂-(alkyl)₀₋₁-aryl;
—S(O)₀₋₂-(alkyl)₀₋₁-aryl;
—S(O)₀₋₂-(alkyl)₀₋₁-heteroaryl;
—S(O)₀₋₂-(alkyl)₀₋₁-(substituted heteroaryl);
—S(O)₀₋₂-(alkyl)₀₋₁-heterocyclyl;
—S(O)₀₋₂-(alkyl)₀₋₁-(substituted heterocyclyl);
-(alkyl)₀₋₁-N(R₆₂₉)₂;
-(alkyl)₀₋₁-NR₆₂₉—CO—O-alkyl;
-(alkyl)₀₋₁-NR₆₂₉—CO-alkyl;
-(alkyl)₀₋₁-NR₆₂₉—CO-aryl;
-(alkyl)₀₋₁-NR₆₂₉—CO-(substituted aryl);
-(alkyl)₀₋₁-NR₆₂₉—CO-heteroaryl;
-(alkyl)₀₋₁-NR₆₂₉—CO-(substituted heteroaryl);
—P(O)(O-alkyl)₂;
—N₃;
-halogen;
-haloalkyl;
-haloalkoxy;
—CO-haloalkyl;
—CO-haloalkoxy;
—NO₂;
—CN;
—OH;
—SH; and in the case of alkyl, alkenyl, and heterocyclyl, oxo;
R₂₂₉ is selected from:
   -hydrogen;
   -alkyl;
   -alkenyl;
   -aryl;
   -substituted aryl;
   -heteroaryl;
   -substituted heteroaryl;
   -alkyl-O-alkyl;
   -alkyl-S-alkyl;
   -alkyl-O-aryl;
   -alkyl-S-aryl:
   -alkyl-O-alkenyl;
   -alkyl-S-alkenyl; and
   -alkyl or alkenyl substituted by one or more substituents selected from:
      —OH;
      -halogen;
      —N(R₆₂₉)₂;
      —CO—N(R₆₂₉)₂;
      —CS—N(R₆₂₉)₂;
      —SO₂—N(R₆₂₉)₂;
      —N₂₉—CO—C₁₋₁₀ alkyl;
      —NR₆₂₉—CS—C₁₋₁₀ alkyl;
      —NR₆₂₉—SO₂—C₁₋₁₀ alkyl;
      —CO—C₁₋₁₀ alkyl;
      —CO—O—C₁₋₁₀ alkyl;
      —N₃;
      -aryl;
      -substituted aryl;
      -heteroaryl;
      -substituted heteroaryl;
      -heterocyclyl;
      -substituted heterocyclyl;
      —CO-aryl;
      —CO-(substituted aryl);
      —CO-heteroaryl; and
      —CO-(substituted heteroaryl);
R₃₂₉ and R₄₂₉ are independently selected from hydrogen, alkyl, alkenyl, halogen, alkoxy, amino, alkylamino, dialkylamino, and alkylthio;
R₅₂₉ is H or C₁₋₁₀ alkyl, or R₅₂₉ can join with X to form a ring that contains one or two heteroatoms;
each R₆₂₉ is independently H or C₁₋₁₀alkyl;
R₇₂₉ is H or C₁₋₁₀ alkyl which may be interrupted by a heteroatom; or when R₁₂₉ is alkyl, R₇₂₉ and R₁₂₉ can join to form a ring;
   and pharmaceutically acceptable salts thereof.

In another embodiment, the IRM compound can be chosen from 1-position ether or thioether substituted 1H-imidazo[4,5-c]pyridin-4-amines defined by Formula XXX below: 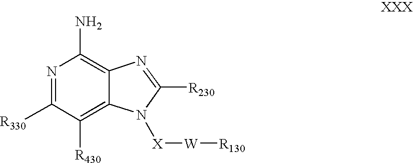
wherein:

X is —CH(R₅₃₀)—, —CH(R₅₃₀)-alkylene-, —CH(R₅₃₀)-alkenylene-, or CH(R₅₃₀)-alkylene-Y-alkylene-;

Y is —O—, or —S(O)₀₋₂—;

—W—R₁₃₀ is selected from —O—R₁₃₀₋₁₋₅ and —S(O)₀₋₂—R₁₃₀₋₆;

R₁₃₀₋₁₋₅ is selected from
—R₆₃₀—C(R₇₃₀)-Z-R₈₃₀-alkyl;
—R₆₃₀—C(R₇₃₀)-Z-R₈₃₀-alkenyl;
—R₆₃₀—C(R₇₃₀)-Z-R₈₃₀-aryl;
—R₆₃₀—C(R₇₃₀)-Z-R₈₃₀-heteroaryl;
—R₆₃₀—C(R₇₃₀)-Z-R₈₃₀-heterocyclyl;
—R₆₃₀—C(R₇₃₀)-Z-H;
—R₆₃₀—N(R₉₃₀)—C(R₇₃₀)—R₈₃₀-alkyl;
—R₆₃₀—N(R₉₃₀)—C(R₇₃₀)—R₈₃₀-alkenyl;
—R₆₃₀—N(R₉₃₀)—C(R₇₃₀)—R₈₃₀-aryl;
—R₆₃₀—N(R₉₃₀)—C(R₇₃₀)—R₈₃₀-heteroaryl;
—R₆₃₀—N(R₉₃₀)—C(R₇₃₀)—R₈₃₀-heterocyclyl;
—R₆₃₀—N(R₉₃₀)—C(R₇₃₀)—R₁₀₃₀;
—R₆₃₀—N(R₉₃₀)—SO₂—R₈₃₀-alkyl;
—R₆₃₀—N(R₉₃₀)—SO₂—R₈₃₀-alkenyl;
—R₆₃₀—N(R₉₃₀)—SO₂—R₈₃₀-aryl;
—R₆₃₀—N(R₉₃₀)—SO₂—R₈₃₀-heteroaryl;
—R₆₃₀—N(R₉₃₀)—SO₂—R₈₃₀-heterocyclyl;
—R₆₃₀—N(R₉₃₀)—SO₂—R₁₀₃₀;
—R₆₃₀—N(R₉₃₀)—SO₂—N(R₅₃₀)—R₈₃₀-alkyl;
—R₆₃₀—N(R₉₃₀)—SO₂—N(R₅₃₀)—R₈₃₀-alkenyl;
—R₆₃₀—N(R₉₃₀)—SO₂—N(R₅₃₀)—R₈₃₀-aryl;
—R₆₃₀—N(R₉₃₀)—SO₂—N(R₅₃₀)—R₈₃₀-heteroaryl;
—R₆₃₀—N(R₉₃₀)—SO₂—N(R₅₃₀)—R₈₃₀-heterocyclyl;
—R₆₃₀—N(R₉₃₀)—SO₂—NH₂;
—R₆₃₀—N(R₉₃₀)—C(R₇₃₀)—N(R₅₃₀)-Q-R₈₃₀-alkyl;
—R₆₃₀—N(R₉₃₀)—C(R₇₃₀)—N(R₅₃₀)-Q-R₈₃₀-alkenyl;
—R₆₃₀—N(R₉₃₀)—C(R₇₃₀)—N(R₅₃₀)-Q-R₈₃₀-aryl;
—R₆₃₀—N(R₉₃₀)—C(R₇₃₀)—N(R₅₃₀)-Q-R₈₃₀-heteroaryl;
—R₆₃₀—N(R₉₃₀)—C(R₇₃₀)—N(R₅₃₀)-Q-R₈₃₀-heterocyclyl;
—R₆₃₀—N(R₉₃₀)—C(R₇₃₀)N(R₅₃₀)₂; 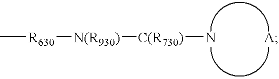
—R₆₃₀—N(R₉₃₀)—C(R₇₃₀)—N(R₁₁₃₀)-Q-R₈₃₀-alkyl;
—R₆₃₀—N(R₉₃₀)—C(R₇₃₀)—N(R₁₁₃₀)-Q-R₈₃₀-alkenyl;
—R₆₃₀—N(R₉₃₀)—C(R₇₃₀)—N(R₁₁₃₀)-Q-R₈₃₀-aryl;
—R₆₃₀—N(R₉₃₀)—C(R₇₃₀)—N(R₁₁₃₀)-Q-R₈₃₀-heteroaryl;
—R₆₃₀—N(R₉₃₀)—C(R₇₃₀)—N(R₁₁₃₀)-Q-R₈₃₀-heterocyclyl;
—R₆₃₀—N(R₉₃₀)—C(R₇₃₀)—N(R₁₁₃₀)H;
-alkenyl;
-aryl;
—R₆₃₀-aryl;
-heteroaryl;
-heterocyclyl;
—R₆₃₀-heteroaryl; and
—R₆₃₀-heterocyclyl;

Z is —N(R₅₃₀)—, —O—, or —S—;

Q is a bond, —CO—, or —SO₂—;

A represents the atoms necessary to provide a 5- or 6-membered heterocyclic or heteroaromatic ring that contains up to three heteroatoms;

R₁₃₀₋₆ is selected from:
-alkyl;
-aryl;
-heteroaryl;
-heterocyclyl;
-alkenyl;
—R₆₃₀-aryl;
—R₆₃₀-heteroaryl; and
—R₆₃₀-heterocyclyl;

each R₅₃₀ is independently hydrogen, C₁₋₁₀ alkyl, or C₂₋₁₀ alkenyl;

R₆₃₀ is alkylene, alkenylene, or alkynylene, which may be interrupted by one or more —O— groups;

R₇₃₀ is ═O or ═S;

R₈₃₀ is a bond, alkylene, alkenylene, or alkynylene, which may be interrupted by one or more —O— groups;

R₉₃₀ is hydrogen, C₁₋₁₀ alkyl, or arylalkyl; or R₉₃₀ can join together with any carbon atom of R₆₃₀ to form a ring of the formula

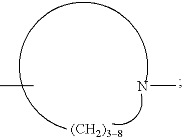

R₁₀₃₀ is hydrogen or C₁₋₁₀ alkyl; or R₉₃₀ and R₁₀₃₀ can join together to form a ring selected from

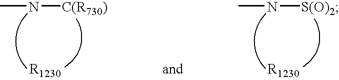

R₁₁₃₀ is C₁₋₁₀ alkyl; or R₉₃₀ and R₁₁₃₀ can join together to form a ring having the structure

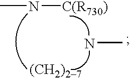

R₁₂₃₀ is C₂₋₇ alkylene which is straight chain or branched, wherein the branching does not prevent formation of the ring; and

R₂₃₀, R₃₃₀ and R₄₃₀ are independently selected from hydrogen and non-interfering substitutents;

and pharmaceutically acceptable salts thereof.
Illustrative non-interfering R₂₃₀ substituents include:
-alkyl;
-alkenyl;
-aryl;
-heteroaryl;
-heterocyclyl;
-alkylene-Y-alkyl;
-alkylene-Y-alkenyl;
-alkylene-Y-aryl; and
-alkyl or alkenyl substituted by one or more substituents selected from the group consisting of:
   —OH;
   -halogen;
   —N(R₅₃₀)₂;
   —C(O)—C₁₋₁₀ alkyl;
   —C(O)—O—C₁₋₁₀ alkyl;
   —N₃;
   -aryl;
   -heteroaryl;
   -heterocyclyl;
   —C(O)-aryl; and
   —C(O)-heteroaryl.

Illustrative non-interfering R₃₃₀ and R₄₃₀ substitutents include:

C₁₋₁₀ alkyl, C₂₋₁₀ alkenyl, C₂₋₁₀ alkynyl, C₁₋₁₀ alkoxy, C₁₋₁₀ alkylthio, amino, alkylamino, dialkylamino, halogen, and nitro.

In another embodiment, the IRM compound can be chosen from 1H-imidazo dimers of the formula (XXXI): 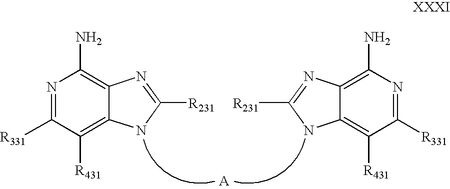
wherein:

A is a divalent linking group selected from the group consisting of:
straight or branched chain C₄₋₂₀ alkylene;
straight or branched chain C₄₋₂₀ alkenylene;
straight or branched chain C₄₋₂₀ alkynylene; and
-Z-Y—W—Y-Z-;

each Z is independently selected from the group consisting of:
straight or branched chain C₂₋₂₀ alkylene;
straight or branched chain C₄₋₂₀ alkenylene; and
straight or branched chain C₄₋₂₀ alkynylene;
any of which may be optionally interrupted by —O—, —N(R₅₃₁)—, or —S(O)₂—;

each Y is independently selected from the group consisting of:
a bond;
—N(R₅₃₁)C(O)—;
—C(O)N(R₅₃₁)—;
—N(R₅₃₁)C(O)N(R₅₃₁)—;
—N(R₅₃₁)S(O)₂—;
—S(O)₂N(R₅₃₁)—;
—OC(O)O—;
—OC(O)—;
—C(O)O—;
—N(R₅₃₁)C(O)O—; and
—OC(O)N(R₅₃₁)—;

W is selected from the group consisting of:
straight or branched chain C₂₋₂₀ alkylene;
straight or branched chain C₂₋₂₀ alkenylene;
straight or branched chain C₄₋₂₀ alkynylene;
straight or branched chain perfluoro C₂₋₂₀ alkylene;
C₁₋₄ alkylene-O—C₁₋₄ alkylene;
—C(O)—;
—S(O)₂—;
—OC(O)O—;
—N(R₅₃₁)C(O)N(R₅₃₁)—; 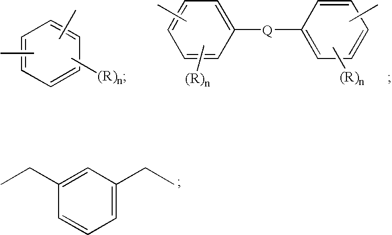
1,5-naphthylene;
2,6-pyridinylene;
1,2-cyclohexylene;
1,3-cyclohexylene;
1,4-cyclohexylene;
trans-1,4-cyclohexylene; 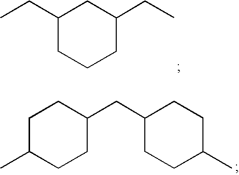
and
trans-5-norbornen-2,3-diyl;
wherein n is 0-4; each R is independently selected from the group consisting of C₁₋₄ alkyl, C₁₋₄ alkoxy, and halogen; and Q is selected from the group consisting of a bond, —CH₂—, and —O—;

R₂₃₁ is selected from the group consisting of:
-hydrogen;
-alkyl;
-alkenyl;
-aryl;
-substituted aryl;
-heteroaryl;
-substituted heteroaryl;
-alkyl-X-alkyl;
-alkyl-X-aryl;
-alkyl-X-alkenyl; and
-alkyl or alkenyl substituted by one or more substituents selected from the group consisting of:
   —OH;
   -halogen;
   —N(R₆₃₁)₂;
   —C(O)—N(R₆₃₁)₂;
   —C(S)—N(R₆₃₁)₂;
   —S(O)₂—N(R₆₃₁)₂;
   —N(R₆₃₁)—C(O)—C₁₋₁₀ alkyl;
   —N(R₆₃₁)—C(S)—C₁₋₁₀ alkyl;
   —N(R₆₃₁)—S(O)₂—C₁₋₁₀ alkyl;
   —C(O)—C₁₋₁₀ alkyl;
   —C(O)—O—C₁₋₁₀ alkyl;
   —N₃;
   -aryl;
   -substituted aryl;
   -heteroaryl;
   -substituted heteroaryl;
   -heterocyclyl;
   -substituted heterocyclyl;
   —C(O)-aryl;
   —C(O)-(substituted aryl);
   —C(O)-heteroaryl; and
   —C(O)-(substituted heteroaryl);

R₃₃₁ and R₄₃₁ are each independently selected from the group consisting of:
-hydrogen;
-halogen;
-alkyl;
-alkenyl;
—X-alkyl; and
—N(R₆₃₁)₂;
or when taken together, R₃₃₁ and R₄₃₁ form a fused aryl or heteroaryl ring that is unsubstituted or substituted by one or more substituents selected from the group consisting of:
   -halogen;
   -alkyl;
   -alkenyl;
   —X-alkyl; and
   —N(R₆₃₁)₂;
or when taken together, R₃₃₁ and R₄₃₁ form a fused 5 to 7 membered saturated ring, containing 0 to 2 heteroatoms and unsubstituted or substituted by one or more substituents selected from the group consisting of:
   -halogen;
   -alkyl;
   -alkenyl;
   —X-alkyl; and
   —N(R₆₃₁)₂;

each R₅₃₁ is independently selected from the group consisting of:
hydrogen;
C₁₋₆ alkyl;
C₃₋₇ cycloalkyl; and
benzyl; or

when Y is —N(R₅₃₁)C(O)—, —C(O)N(R₅₃₁)—, —N(R₅₃₁)C(O)N(R₅₃₁)—, —N(R₅₃₁)S(O)₂—, —S(O₂)N(R₅₃₁)—, —N(R₅₃₁)C(O)O—, or —OC(O)N(R₅₃₁)— and the nitrogen of the N(R₅₃₁) group is bonded to Z, then R₅₃₁ can join with Z to form a ring having the structure

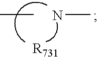

each R₆₃₁ is independently hydrogen or C₁₋₁₀ alkyl;

R₇₃₁ is C₃₋₈ alkylene; and

X is —O— or —S—;

with the proviso that if W is —C(O)—, —S(O)₂—, —OC(O)O—, or —N(R₅₃₁)C(O)N(R₅₃₁)— then each Y is a bond;

and pharmaceutically acceptable salts thereof.

In another embodiment, the IRM compound can be chosen from 6-, 7-, 8-, or 9-position aryl or heteroaryl substituted 1H-imidazo[4,5-c]quinolin-4-amines of the following Formula (XXXII): 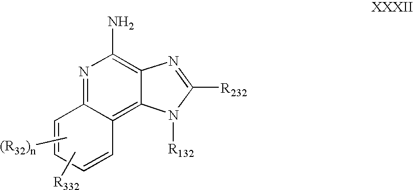
wherein:

R₃₂ is selected from the group consisting of alkyl, alkoxy, hydroxy, and trifluoromethyl;

n is 0 or 1;

R₁₃₂ and R₂₃₂ are independently selected from the group consisting of hydrogen and non-interfering substitutents;

R₃₃₂ is selected from the group consisting of:
-Z-Ar,
-Z-Ar′—Y—R₄₃₂,
-Z-Ar′—X—Y—R₄₃₂,
-Z-Ar′—R₅₃₂, and
-Z-Ar′—X—R₅₃₂;

Ar is selected from the group consisting of aryl and heteroaryl both of which can be unsubstituted or can be substituted by one or more substituents independently selected from the group consisting of alkyl, alkenyl, alkoxy, methylenedioxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkoxy, heteroaryl, heteroaryloxy, heteroarylalkoxy, heterocyclyl, heterocyclylalkyl, amino, alkylamino, and dialkylamino;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈₃₂)—,
—C(R₆₃₂)—,
—C(R₆₃₂)—O—,
—O—C(R₆₃₂)—,
—O—C(O)—O—,
—N(R₈₃₂)-Q-,
—C(R₆₃₂)—N(R₈₃₂)—,
—O—C(R₆₃₂)—N(R₈₃₂)—,
—C(R₆₃₂)—N(OR₉₃₂)—,

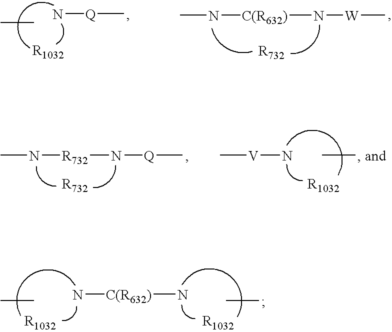

Z is selected from the group consisting of a bond, alkylene, alkenylene, and alkynylene;

R₄₃₂ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅₃₂ is selected from the group consisting of:

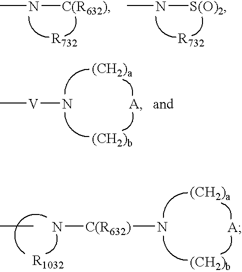

each R₆₃₂ is independently selected from the group consisting of ═O and ═S;

each R₇₃₂ is independently C₂₋₇ alkylene;

each R₈₃₂ is independently selected from the group consisting of hydrogen, alkyl, alkoxyalkylenyl, and arylalkylenyl;

R₉₃₂ is selected from the group consisting of hydrogen and alkyl;

each R₁₀₃₂ is independently C₃₋₈ alkylene;

A is selected from the group consisting of —O—, —C(O)—, —S(O)₀₋₂—, —CH₂—, and —N(R₄₃₂)—;

Q is selected from the group consisting of a bond, —C(R₆₃₂)—, —C(R₆₃₂)—C(R₆₃₂), —S(O)₂—, —C(R₆₃₂)—N(R₈₃₂)—W—, —S(O)₂—N(R₈₃₂)—, —C(R₆₃₂)—O—, and —C(R₆₃₂)—N(OR₉₃₂)—;

V is selected from the group consisting of —C(R₆₃₂)—, —O—C(R₆₃₂)—, —N(R₈₃₂)—C(R₆₃₂)—, and —S(O)₂—;

W is selected from the group consisting of a bond, —C(O)—, and —S(O)₂—; and

a and b are independently integers from 1 to 6 with the proviso that a+b is ≦7;

and pharmaceutically acceptable salts thereof.

Illustrative non-interfering R₁₃₂ substituents include:
—R₄₃₂,
—X—R₄₃₂,
—X—Y—R₄₃₂,
—X—Y—X—Y—R₄₃₂, and
—X—R₅₃₂;

wherein:

each X is independently selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

each Y is independently selected from the group consisting of:
—S(O)₀₋₂—,
—S(O)₂—N(R₈₃₂)—,
—C(R₆₃₂)—,
—C(R₆₃₂)—O—,
—O—C(R₆₃₂)—,
—O—C(O)—O—,
—N(R₈₃₂)-Q-,
—C(R₆₃₂)—N(R₈₃₂)—,
—O—C(R₆₃₂)—N(R₈₃₂)—,
—C(R₆₃₂)—N(OR₉₃₂)—,

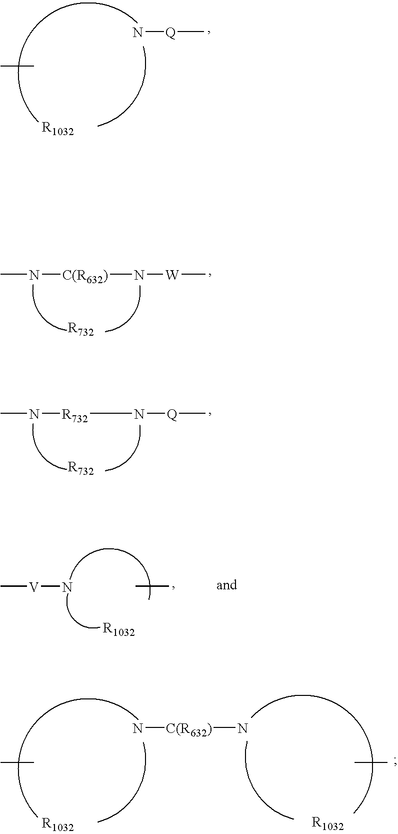

R₄₃₂ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅₃₂ is selected from the group consisting of:

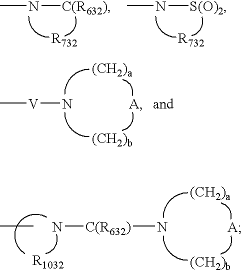

each R₆₃₂ is independently selected from the group consisting of ═O and ═S;

each R₇₃₂ is independently C₂₋₇ alkylene;

each R₈₃₂ is independently selected from the group consisting of hydrogen, alkyl, alkoxyalkylenyl, and arylalkylenyl;

each R₉₃₂ is independently selected from the group consisting of hydrogen and alkyl;

each R₁₀₃₂ is independently C₃₋₈ alkylene;

A is selected from the group consisting of —O—, —C(O)—, —S(O)₀₋₂—, —CH₂—, and —N(R₄₃₂)—;

each Q is independently selected from the group consisting of a bond, —C(R₆₃₂)—, —C(R₆₃₂)—C(R₆₃₂)—, —S(O)₂—, —C(R₆₃₂)—N(R₈₃₂)—W—, —S(O)₂—N(R₈₃₂)—, —C(R₆₃₂)—O—, and —C(R₆₃₂)—N(OR₉₃₂)—;

each V is independently selected from the group consisting of —C(R₆₃₂)—, —O—C(R₆₃₂)—, —N(R₈₃₂)—C(R₆₃₂)—, and —S(O)₂—;

each W is independently selected from the group consisting of a bond, —C(O)—, and —S(O)₂—; and

a and b are independently integers from 1 to 6 with the proviso that a+b is ≦7;

Illustrative non-interfering R₂₃₂ substitutents include:
—R₄₃₂,
—X—R₄₃₂,
—X—Y—R₄₃₂, and
—X—R₅₃₂;

wherein:

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of:
—S(O)₀₋₂—,
—S(O)₂—N(R₈₃₂)—,
—C(R₆₃₂)—,
—C(R₆₃₂)—O—,
—O—C(R₆₃₂)—,
—O—C(O)—O—,
—N(R₈₃₂)-Q-,
—C(R₆₃₂)—N(R₈₃₂)—,
—O—C(R₆₃₂)—N(R₈₃₂)—,
—C(R₆₃₂)—N(OR₉₃₂)—,

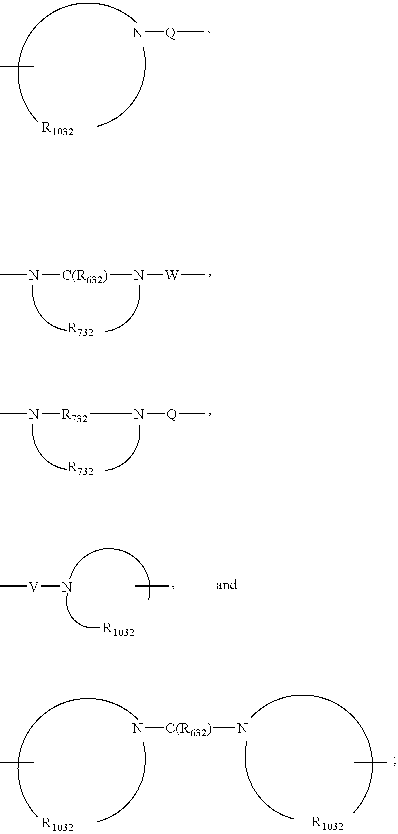

R₄₃₂ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅₃₂ is selected from the group consisting of:

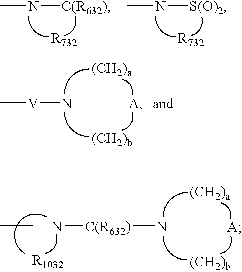

each R₆₃₂ is independently selected from the group consisting of ═O and ═S;

each R₇₃₂ is independently C₂₋₇ alkylene;

each R₈₃₂ is independently selected from the group consisting of hydrogen, alkyl, alkoxyalkylenyl, and arylalkylenyl;

R₉₃₂ is selected from the group consisting of hydrogen and alkyl;

each R₁₀₃₂ is independently C₃₋₈ alkylene;

A is selected from the group consisting of —O—, —C(O)—, —S(O)₀₋₂—, —CH₂—, and —N(R₄₃₂)—;

Q is selected from the group consisting of a bond, —C(R₆₃₂)—, —C(R₆₃₂)—C(R₆₃₂)—, —S(O)₂—, —C(R₆₃₂)—N(R₈₃₂)—W—, —S(O)₂—N(R₈₃₂)—, —C(R₆₃₂)—O—, and —C(R₆₃₂)—N(OR₉₃₂)—;

V is selected from the group consisting of —C(R₆₃₂)—, —O—C(R₆₃₂)—, —N(R₈₃₂)—C(R₆₃₂)—, and —S(O)₂—;

W is selected from the group consisting of a bond, —C(O)—, and —S(O)₂—; and

a and b are independently integers from 1 to 6 with the proviso that a+b is ≦7;

In another embodiment, the IRM compound can be chosen from aryloxy or arylalkyleneoxy substituted 1H-imidaz[4,5-c]quinoline-4-amines of the following Formula XXXIII: 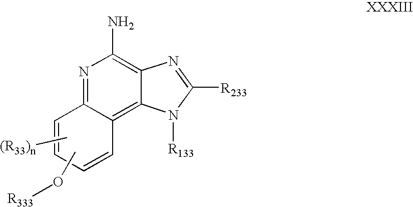
wherein:

R₃₃₃ is selected from the group consisting of:
-Z-Ar,
-Z-Ar′—Y—R₄₃₃,
-Z-Ar′—X—Y—R₄₃₃,
-Z-Ar′—R₅₃₃, and
-Z-Ar′—X—R₅₃₃;

Z is selected from the group consisting of a bond, alkylene, alkenylene, and alkynylene wherein alkylene, alkenylene, and alkynylene are optionally interrupted with —O—;

Ar is selected from the group consisting of aryl and heteroaryl both of which can be unsubstituted or can be substituted by one or more substituents independently selected from the group consisting of alkyl, alkenyl, alkoxy, methylenedioxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

Ar′ is selected from the group consisting of arylene and heteroarylene both of which can be unsubstituted or can be substituted by one or more substituents independently selected from the group consisting of alkyl, alkenyl, alkoxy, haloalkyl, haloalkoxy, halogen, nitro, hydroxy, hydroxyalkyl, mercapto, cyano, carboxy, formyl, aryl, aryloxy, arylalkyleneoxy, heteroaryl, heteroaryloxy, heteroarylalkyleneoxy, heterocyclyl, heterocyclylalkylenyl, amino, alkylamino, and dialkylamino;

R₃₃ is selected from the group consisting of alkyl, alkoxy, hydroxy, halogen, and trifluoromethyl;

n is 0 or 1;

R₁₃₃ is selected from the group consisting of:
—R₄₃₃,
—X—R₄₃₃,
—X—Y—R₄₃₃,
—X—Y—X—Y—R₄₃₃, and
—X—R₅₃₃;

R₂₃₃ is selected from the group consisting of:
—R₄₃₃,
—X—R₄₃₃,
—X—Y—R₄₃₃, and
—X—R₅₃₃;

each X is independently selected from the group consisting of alkylene, alkenylene, alkynylene, arylene, heteroarylene, and heterocyclylene wherein the alkylene, alkenylene, and alkynylene groups can be optionally interrupted by arylene, heteroarylene or heterocyclylene or by one or more —O— groups;

each Y is independently selected from the group consisting of:
—S(O)₀₋₂—,
—S(O)₂—N(R₈₃₃)—,
—C(R₆₃₃)—,
—C(R₆₃₃)—O—,
—O—C(R₆₃₃)—,
—O—C(O)—O—,
—N(R₈₃₃)-Q-,
—C(R₆₃₃)—N(R₈₃₃)—,
—O—C(R₆₃₃)—N(R₈₃₃)—,
—C(R₆₃₃)—N(OR₉₃₃)—,

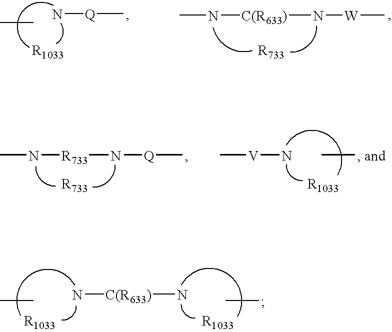

each R₄₃₃ is independently selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

each R₅₃₃ is independently selected from the group consisting of:

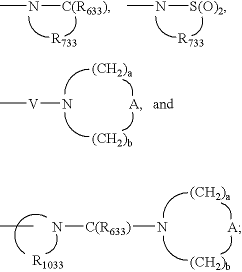

each R₆₃₃ is independently selected from the group consisting of ═O and ═S;

each R₇₃₃ is independently C₂₋₇ alkylene;

each R₈₃₃ is independently selected from the group consisting of hydrogen, alkyl, alkoxyalkylenyl, and arylalkylenyl;

each R₉₃₃ is independently selected from the group consisting of hydrogen and alkyl;

each R₁₀₃₃ is independently C₃₋₈ alkylene;

each A is independently selected from the group consisting of —O—, —C(O)—, —S(O)₀₋₂—, —CH₂—, and —N(R₄₃₃)—;

each Q is independently selected from the group consisting of a bond, —C(R₆₃₃)—, —C(R₆₃₃)—C(R₆₃₃)—, —S(O)₂—, —C(R₆₃₃)—N(R₈₃₃)—W—, —S(O)₂—N(R₈₃₃)—, —C(R₆₃₃)—O—, and —C(R₆₃₃)—N(OR₉₃₃)—;

each V is independently selected from the group consisting of —C(R₆₃₃)—, —O—C(R₆₃₃)—, —N(R₈₃₃)—C(R₆₃₃)—, and —S(O)₂—;

each W is independently selected from the group consisting of a bond, —C(O)—, and —S(O)₂—; and

a and b are independently integers from 1 to 6 with the proviso that a+b is ≦7;

or a pharmaceutically acceptable salt thereof.

In another embodiment, the IRM compound can be chosen from 1H-imidaz[4,5-c]quinoline-4-amines of the following Formula XXXIV: 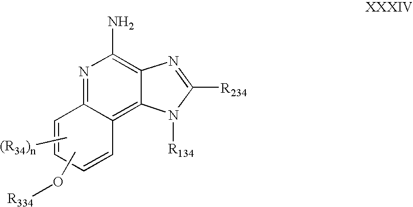
wherein:

R₃₃₄ is selected from the group consisting of
-Z-Y—R₄₃₄,
-Z-Y—X—Y—R₄₃₄,
-Z-R₅₃₄,
-Z-Het,
-Z-Het′-R₄₃₄, and
-Z-Het′-Y—R₄₃₄;

Z is selected from the group consisting of alkylene, alkenylene, and alkynylene, wherein alkylene, alkenylene, and alkynylene can be optionally interrupted with one or more —O— groups;

R is selected from the group consisting of alkyl, alkoxy, hydroxy, halogen, and trifluoromethyl;

n is 0 or 1;

R₁ is selected from the group consisting of
—R₄₃₄,
—X—R₄₃₄,
—X—Y—R₄₃₄,
—X—Y—X—Y—R₄₃₄, and
—X—R₅₃₄;

R₂₃₄ is selected from the group consisting of
—R₄₃₄,
—X—R₄₃₄,
—X—Y—R₄₃₄, and
—X—R₅₃₄;

X is selected from the group consisting of alkylene, alkenylene, alkynylene, arylene, heteroarylene, and heterocyclylene wherein the alkylene, alkenylene, and alkynylene groups can be optionally interrupted or terminated with arylene, heteroarylene, or heterocyclylene, and optionally interrupted by one or more —O— groups;

Y is selected from the group consisting of
—S(O)₀₋₂—,
—S(O)₂—N(R₈₃₄)—,
—C(R₆₃₄)—,
—C(R₆₃₄)—O—,
—O—C(R₆₃₄)—,
—O—C(O)—O—,
—N(R₈₃₄)-Q-,
—C(R₆₃₄)—N(R₈₃₄)—,
—O—C(R₆₃₄)—N(R₈₃₄)—,
—C(R₆₃₄)—N(OR₉₃₄)—,

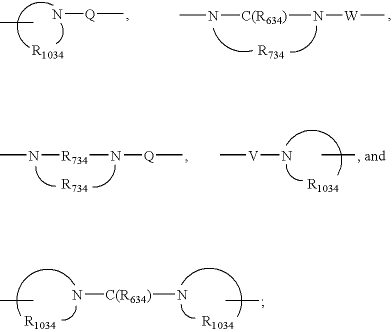

R₄₃₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, (dialkylamino)alkyleneoxy, and in the case of alkyl, alkenyl, alkynyl, and heterocyclyl, oxo;

R₅₃₄ is selected from the group consisting of

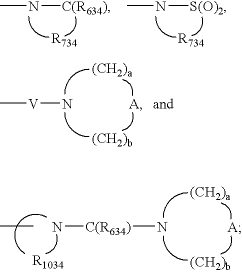

R₆₃₄ is selected from the group consisting of ═O and ═S;

R₇₃₄ is C₂₋₇ alkylene;

R₈₃₄ is selected from the group consisting of hydrogen, alkyl, alkoxyalkylenyl, and arylalkylenyl;

R₉₃₄ is selected from the group consisting of hydrogen and alkyl;

R₁₀₃₄ is C₃₋₈ alkylene;

A is selected from the group consisting of —O—, —C(O)—, —S(O)₀₋₂—, and —N(R₄₃₄)—;

Het is heterocyclyl which can be unsubstituted or substituted by one or more substituents independently selected from the group consisting of alkyl, alkoxy, haloalkyl, haloalkoxy, halogen, nitro, hydroxy, hydroxyalkyl, mercapto, cyano, aryloxy, arylalkyleneoxy, heteroaryloxy, heteroarylalkyleneoxy, heterocyclyl, hydroxyalkyleneoxyalkylenyl, amino, alkylamino, dialkylamino, (dialkylamino)alkyleneoxy, and oxo;

Het′ is heterocyclylene which can be unsubstituted or substituted by one or more substituents independently selected from the group consisting of alkyl, alkoxy, haloalkyl, haloalkoxy, halogen, nitro, hydroxy, hydroxyalkyl, mercapto, cyano, aryloxy, arylalkyleneoxy, heteroaryloxy, heteroarylalkyleneoxy, amino, alkylamino, dialkylamino, (dialkylamino)alkyleneoxy, and oxo;

Q is selected from the group consisting of a bond, —C(R₆₃₄)—, —C(R₆₃₄)—C(R₆₃₄)—, —S(O)₂—, —C(R₆₃₄)—N(R₈₃₄)—W—, —S(O)₂—N(R₈₃₄)—, —C(R₆₃₄)—O—, and —C(R₆₃₄)—N(OR₉₃₄)—;

V is selected from the group consisting of —C(R₆₃₄)—, —O—C(R₆₃₄)—, —N(R₈₃₄)—C(R₆₃₄)—, and —S(O)₂—;

W is selected from the group consisting of a bond, —C(O)—, and —S(O)₂—; and

a and b are independently integers from 1 to 6 with the proviso that a+b is ≦7;

with the proviso that Z can also be a bond when:
R₃₃₄ is -Z-Het, -Z-Het′-R₄₃₄, or -Z-Het′-Y—R₄₃₄; or
R₃₃₄ is -Z-Y—R₄₃₄ or -Z-Y—X—Y—R₄₃₄, and Y is selected from —S(O)₀₋₂—, —S(O)₂—N(R₈₃₄)—, —C(R₆₃₄)—, —C(R₆₃₄)—O—, —C(R₆₃₄)—N(R₈₃₄)—, 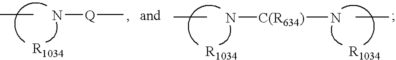
R₃₃₄ is -Z-R₅₃₄ and R₅₃₄ is 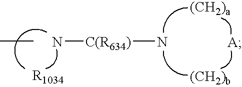
   or a pharmaceutically acceptable salt thereof.

Herein, “non-interfering” means that the ability of the compound or salt to modulate (e.g., induce or inhibit) the biosynthesis of one or more cytokines is not destroyed by the non-interfering substituent.

As used herein, the terms “alkyl,” “alkenyl,” “alkynyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

Unless otherwise specified, “alkylene,” “alkenylene,” and “alkynylene” are the divalent forms of the “alkyl,” “alkenyl,” and “alkynyl” groups defined above. For example, an arylalkenyl group comprises an alkylene moiety to which an aryl group is attached.

The term “haloalkyl” is inclusive of groups that are substituted by one or more halogen atoms, including perfluorinated groups. This is also true of other groups that include the prefix “halo-.” Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl, and indenyl.

The term “hetero atom” refers to the atoms O, S, or N.

The term “heteroaryl” includes aromatic rings or ring systems that contain at least one ring hetero atom. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring hetero atom and includes all of the fully saturated and partially unsaturated derivatives of the above mentioned heteroaryl groups. Exemplary heterocyclic groups include pyrrolidinyl, tetrahydrofuranyl, morpholinyl, thiomorpholinyl, piperidinyl, piperazinyl, thiazolidinyl, imidazolidinyl, isothiazolidinyl, tetrahydropyranyl, quinuclidinyl, homopiperidinyl, homopiperazinyl, and the like.

The terms “arylene,” “heteroarylene,” and “heterocyclylene” are the divalent forms of the “aryl,” “heteroaryl,” and “heterocyclyl” groups defined above. Likewise, “arylenyl,” “heteroarylenyl,” and “heterocyclylenyl” are the divalent forms of the “aryl,” “heteroaryl,” and “heterocyclyl” groups defined above. For example, an alkylarylenyl group comprises an arylene moiety to which an alkyl group is attached.

Unless otherwise specified, the aryl, heteroaryl, and heterocyclyl groups of Formulas IX-XXXIV can be unsubstituted or substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylthio, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, heterocyclylcarbonyl, aryloxycarbonyl, heteroaryloxycarbonyl, arylthiocarbonyl, heteroarylthiocarbonyl, alkanoyloxy, alkanoylthio, alkanoylamino, aroyloxy, aroylthio, aroylamino, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonyl, dialkylaminocarbonyl, arylaminocarbonyl, arylalkylaminocarbonyl, alkenylaminocarbonyl, heteroarylaminocarbonyl, heteroarylalkylaminocarbonyl, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If any other groups are identified as being “substituted” or “optionally substituted,” then those groups can also be substituted by one or more of the above enumerated substituents.

The IRM compounds and salts thereof described herein include any of their pharmaceutically acceptable forms, such as isomers (e.g., diastereomers and enantiomers), solvates, polymorphs, and the like. In particular, if a compound is optically active, the invention specifically includes the use of each of the compound's enantiomers as well as racemic mixtures of the enantiomers.

In some applications, for example, the preferred IRM compound is other than imiquimod or S-28463 (i.e., resiquimod: 4-Amino-α,α-dimethyl-1H-imidazo[4,5-c]quinoline-1-ethanol).

Examples of particular IRM compounds include 2-propyl[1,3]thiazolo[4,5-c]quinolin-4-amine, which is considered predominantly a TLR8 agonist (and not a substantial TLR7 agonist), 4-amino-α,α-dimethyl-1H-imidazo[4,5-c]quinoline-1-ethanol, which is considered predominantly a TLR7 agonist (and not a substantial TLR8 agonist), and 4-amino-2-(ethoxymethyl)-α,α-dimethyl-6,7,8,9-tetrahydro-1H-imidazo[4,5-c]quinoline-1-ethanol, which is a TLR7 and TLR8 agonist. In addition to its TLR7 activity (and low TLR8 activity), 4-amino-α,α-dimethyl-1H-imidazo[4,5-c]quinoline-1-ethanol has beneficial characteristics, including that it has a much lower CNS effect when delivered systemically compared to imiquimod. Other examples of specific IRM compounds include, e.g., N-[4-(4-amino-2-butyl-1H-imidazo[4,5-c][1,5]naphthyridin-1-yl)butyl]-N′-cyclohexylurea, 2-methyl-1-(2-methylpropyl)-1H-imidazo[4,5-c][1,5]naphthyridin-4-amine, 1-(2-methylpropyl)-1H-imidazo[4,5-c][1,5]naphthyridin-4-amine, N-{2-[4-amino-2-(ethoxymethyl)-1H-imidazo[4,5-c]quinolin-1-yl]-1,1-dimethylethyl}methanesulfonamide, N-[4-(4-amino-2-ethyl-1H-imidazo[4,5-c]quinolin-1-yl)butyl]methanesulfonamide, 2-methyl-1-[5-(methylsulfonyl)pentyl]-1H-imidazo[4,5-c]quinolin-4-amine, N-[4-(4-amino-2-propyl-1H-imidazo[4,5-c]quinolin-1-yl)butyl]methanesulfonamide, 2-butyl-1-[3-(methylsulfonyl)propyl]-1H-imidazo[4,5-c]quinoline-4-amine, 2-butyl-1-{2-[(1-methylethyl)sulfonyl]ethyl}-1H-imidazo[4,5-c]quinolin-4-amine, N-{2-[4-amino-2-(ethoxymethyl)-1H-imidazo[4,5-c]quinolin-1-yl]-1,1-dimethylethyl}-N′-cyclohexylurea, N-{2-[4-amino-2-(ethoxymethyl)-1H-imidazo[4,5-c]quinolin-1-yl]-1,1-dimethylethyl}cyclohexanecarboxamide, N-{2-[4-amino-2-(ethoxymethyl)-1H-imidazo[4,5-c]quinolin-1-yl]ethyl}-N′-isopropylurea. Resiquimod, 4-amino-2-ethoxymethyl-α,α-dimethyl-1H-imidazo[4,5-c]quinoline-1-ethanol, may also be used in certain situations where a combination TLR 7 and TLR 8 agonist is desired.

Exemplary Applications

Soluble IRM-polymer complexes can be used in a wide variety of applications, such as in the treatment of a wide variety of conditions. For example, IRMs such as imiquimod—a small molecule, imidazoquinoline IRM, marketed as ALDARA (3M Pharmaceuticals, St. Paul, Minn.)—have been shown to be useful for the therapeutic treatment of warts, as well as certain cancerous or pre-cancerous lesions (See, e.g., Geisse et al., J. Am. Acad. Dernatol., 47(3): 390-398 (2002); Shumack et al., Arch. Dermatol., 138: 1163-1171 (2002); U.S. Pat. No. 5,238,944 and International Publication No. WO 03/045391.

Conditions that may be treated by administering a soluble IRM-polymer complex of the present invention include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia areata; and

(g) diseases associated with wound repair such as, for example, inhibition of keloid formation and other types of scarring (e.g., enhancing wound healing, including chronic wounds).

Additionally, a soluble IRM-polymer complex of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Certain soluble IRM-polymer complexes of the present invention may be particularly helpful in individuals having compromised immune function. For example, certain complexes may be used for treating the opportunistic infections and tumors that occur after suppression of cell mediated immunity in, for example, transplant patients, cancer patients and HIV patients.

The soluble IRM-polymer complexes of the invention may be particularly beneficial for targeting to solid tumors and cancerous organs or tissue regions. If the residence time of the IRM is extended within the cancerous tissue, it is believed that the body's immune response to the cancer can be enhanced and directly targeted to relevant tumor antigens. This not only may help reduce or eliminate cancer at the targeted site of IRM preparation delivery, but, by sensitizing the immune system to the cancer, may help the immune system attack the cancer in other locations throughout the body. This approach to treatment may be used alone or in conjunction with other treatments for the cancer, such as therapeutic cancer vaccination, antibody-based therapies such as Rituxan and Herceptin, and other chemotherapies.

Examples of cancers that may be particularly suitable for targeting of a soluble IRM-polymer complex to a localized tissue region include, but are not limited to, breast cancer, lung cancer, stomach cancer, head and neck cancer, colorectal cancer, renal cell carcinoma, pancreatic cancer, basal cell carcinoma, cervical cancer, melanoma, prostate cancer, ovarian cancer, and bladder cancer.

The methods, materials, and articles of the present invention may be applicable for any suitable subject. Suitable subjects include, but are not limited to, animals such as, but not limited to, humans, non-human primates, rodents, dogs, cats, horses, pigs, sheep, goats, cows, or birds. IRMs may also be particularly helpful in individuals having compromised immune functioning, such as those with HIV AIDS, transplant patients, and cancer patients.

An amount of an IRM-polymer complex effective for a given therapeutic or prophylactic application is an amount sufficient to achieve the intended therapeutic or prophylactic application. The precise amount of IRM-polymer complex used will vary according to factors known in the art including, but not limited to, the physical and chemical nature of the IRM compound, the physical and chemical matter of the polymer, the nature of the composition, the intended dosing regimen, the state of the subject's immune system (e.g., suppressed, compromised, stimulated), the method of administering the IRM-polymer complex, and the species to which the IRM-polymer complex is being administered. Accordingly it is not practical to set forth generally the amount that constitutes an amount of IRM and IRM-polymer complex effective for all possible applications. Those of ordinary skill in the art, however, can readily determine an appropriate amount with due consideration of such factors.

### EXAMPLES

The following examples are presented merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Preparation of N-{2-[4-amino-7-(6-aminohexyloxy)-2-ethoxymethyl-1H-imidazo[4,5-c]quinolin-1-yl]-1,1-dimethylethyl}methanesulfonamide

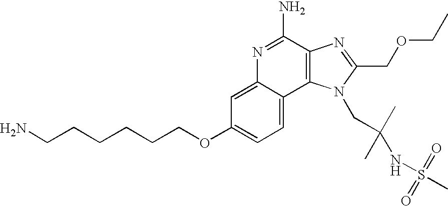 Part A

A mixture of triethyl orthoformate (92 milliliters (mL), 0.55 mole (mol)) and 2,2-dimethyl-[1,3]-dioxane-4,6-dione (75.3 g, 0.522 mol) (Meldrum's acid) was heated at 55° C. for 90 minutes and then cooled to 45° C. A solution of 3-benzyloxyaniline (100.2 g, 0.5029 mol) in methanol (200 mL) was slowly added to the reaction over a period of 45 minutes while maintaining the reaction temperature below 50° C. The reaction was then heated at 45° C. for one hour, allowed to cool to room temperature, and stirred overnight. The reaction mixture was cooled to 1° C., and the product was isolated by filtration and washed with cold ethanol (˜400 mL) until the filtrate was colorless. 5-{[(3-benzyloxy)phenylimino]methyl}-2,2-dimethyl-[1,3]-dioxane-4,6-dione (170.65 g) was isolated as a tan, powdery solid.

Part B

A mixture of 5-{[(3-benzyloxy)phenylimino]methyl}-2,2-dimethyl-[1,3]-dioxane-4,6-dione (170.65 g, 0.483 mol) and DOWTHERM A (800 mL) was heated to 100° C. and then slowly added to a flask containing DOWTHERM A (1.3 L, heated at 210° C.) over a period of 40 minutes. During the addition, the reaction temperature was not allowed to fall below 207° C. Following the addition, the reaction was stirred at 210° C. for one hour, and then allowed to cool to ambient temperature. A precipitate formed, which was isolated by filtration, washed with diethyl ether (1.7 L) and acetone (0.5 liter (L)), and dried in an oven to provide 76.5 grams (g) of 7-benzyloxyquinolin-4-ol as a tan powder.

Part C

A mixture of 7-benzyloxyquinolin-4-ol (71.47 g, 0.2844 mol) and propionic acid (700 mL) was heated to 125° C. with vigorous stirring. Nitric acid (23.11 mL of 16 molar (M)) was slowly added over a period of 30 minutes while maintaining the reaction temperature between 121° C. and 125° C. After the addition, the reaction was stirred at 125° C. for 1 hour then allowed to cool to ambient temperature. The resulting solid was isolated by filtration, washed with water, and dried in an oven for 1.5 days to provide 69.13 g of 7-benzyloxy-3-nitroquinolin-4-ol as a grayish powder.

Part D

N,N-Dimethylformamide (100 mL) (DMF) was cooled to 0° C., and phosphorous oxychloride (27.5 mL, 0.295 mol) was added dropwise. The resulting solution was stirred for 25 minutes and then added dropwise to a mixture of 7-benzyloxy-3-nitroquinolin-4-ol (72.87 g, 0.2459 mol) in DMF (400 mL). Following the addition, the reaction was heated at 100° C. for 5 minutes, cooled to ambient temperature, and poured into ice water with stirring. A tan precipitate formed, which was isolated by filtration and dissolved in dichloromethane. The resulting solution was dried over magnesium sulfate, filtered, and concentrated under reduced pressure to yield 72.9 g of 7-benzyloxy-4-chloro-3-nitroquinoline as a light brown solid.

Part E

Triethylamine (12.8 mL, 92.0 millimole (mmol)) and 1,2-diamino-2-methylpropane (5.29 mL, 50.6 mmol) were added sequentially to a solution of 7-benzyloxy-4-chloro-3-nitroquinoline (14.5 g, 46.0 mmol) in dichloromethane (400 mL). The reaction mixture was stirred overnight and then concentrated under reduced pressure. The residue was partitioned between water (200 mL) and dichloromethane (300 mL). The organic layer was washed with brine, dried over sodium sulfate, and then concentrated under reduced pressure to provide crude product as a brown solid. The crude product was passed through a layer of silica gel (eluting sequentially with chloroform and 96:4 chloroform:methanol) to provide 12.4 g of (2-amino-2-methylpropyl)(7-benzyloxy-3-nitroquinolin-4-yl)amine as a yellow solid.

Part F

Under a nitrogen atmosphere, a solution of (2-amino-2-methylpropyl)(7-benzyloxy-3-nitroquinolin-4-yl)amine (12.4 g, 33.9 mmol) in dichloromethane (400 mL) was cooled to 0° C. Triethylamine (9.43 mL, 67.8 mmol) and methanesulfonic anhydride (5.90 g, 33.9 mmol) were sequentially added, and the reaction was stirred at ambient temperature for two hours. An analysis by HPLC indicated that the reaction was incomplete, and additional methanesulfonic anhydride (1.4 g, 8.0 mmol) was added. The reaction was stirred for an additional 90 minutes, and additional methanesulfonic anhydride (0.7 g, 4 mmol) was added. The reaction was stirred for an additional three hours, and saturated aqueous sodium bicarbonate (200 mL) was added. A precipitate began to form in the organic layer, which was separated and concentrated under reduced pressure to provide a yellow solid. The solid was triturated with water (200 mL) with heating, isolated by filtration, washed with water (3×100 mL) and diethyl ether (3×50 mL), and dried overnight under vacuum to provide 14.8 g of N-[1,l-dimethyl-2-(3-nitro-7-benzyloxyquinolin-4-ylamino)ethyl]methanesulfonamide as a yellow powder.

Part G

N-[1,1-Dimethyl-2-(3-nitro-7-benzyloxyquinolin-4-ylamino)ethyl]methanesulfonamide (14.8 g, 33.3 mmol) was mixed with acetonitrile (300 mL) and added to a Parr flask; 5% platinum on carbon (2 g) was added. The reaction was flushed with nitrogen and placed under hydrogen pressure (40 pounds per square inch (psi), 2.8×10⁵ Pascals (Pa)) for 5.5 hours with the hydrogen replaced after two hours. An analysis by TLC indicated the presence of starting material. Additional acetonitrile (200 mL) and 5% platinum on carbon (2 g) were added, and the reaction was placed under hydrogen pressure overnight. The reaction mixture was filtered through a layer of CELITE filter aid, and the filter cake was washed with acetonitrile. The filtrate was concentrated under reduced pressure. Toluene and dichloromethane were added and removed under reduced pressure twice to yield 12.6 g of N-[2-(3-amino-7-benzyloxyquinolin-4-ylamino)-1,1-dimethylethyl]methanesulfonamide as a solid.

Part H

Under a nitrogen atmosphere, a solution of N-[2-(3-amino-7-benzyloxyquinolin-4-ylamino)-1,1-dimethylethyl]methanesulfonamide (12.6 g, 30.4 mmol) in dichloromethane (300 mL) was cooled to ˜0° C.; triethylamine (4.23 mL, 30.4 mmol) was added. Ethoxy acetyl chloride (3.33 mL, 30.4 mmol) was added dropwise, and the reaction was stirred at ambient temperature for 1.5 hours. The volatiles were removed under reduced pressure, and the residue was dissolved in ethanol (300 mL). Triethylamine (13 mL) was added, and the reaction was heated at reflux overnight and allowed to cool to ambient temperature. The volatiles were removed under reduced pressure. The residue was dissolved in dichloromethane (300 mL), and the resulting solution was washed with water (2×100 mL) and brine, dried over sodium sulfate, filtered, and concentrated under reduced pressure to provide a brown oil. The oil was purified by column chromatography on silica gel (eluting with 97.5:2.5 chloroform:methanol) to provide 12.4 g of N-[2-(7-benzyloxy-2-ethoxymethyl-1H-imidazo[4,5-c]quinolin-1-yl)-1,1-dimethylethyl]methanesulfonamide as a beige solid.

Part I

A solution of N-[2-(7-benzyloxy-2-ethoxymethyl-1H-imidazo[4,5-c]quinolin-1-yl)-1,1-dimethylethyl]methanesulfonamide (9.38 g, 19.5 mmol) in ethanol (150 mL) was added to a Parr vessel containing 10% palladium on carbon (0.83 g). The reaction was placed under hydrogen pressure (50 psi, 3.4×10⁵ Pa) over two nights. Starting material remained as evidenced by a TLC analysis, and additional 10% palladium on carbon (1.02 g) was added. The reaction was continued for an additional eight hours. The reaction mixture was filtered through a layer of CELITE filter aid, and the filter cake was washed with ethanol and methanol. The filtrate was concentrated under reduced pressure, and the residue was dissolved in toluene and concentrated under reduced pressure several times to yield a yellow powder, which was dried under high vacuum to provide 7.37 g of N-[2-(2-ethoxymethyl-7-hydroxy-1H-imidazo[4,5-c]quinolin-1-yl)-1,1-dimethylethyl]methanesulfonamide as a yellow solid.

Part J

Under a nitrogen atmosphere, cesium carbonate (9.18 g, 28.2 mmol) was added in a single portion to a solution of N-[2-(2-ethoxymethyl-7-hydroxy-1H-imidazo[4,5-c]quinolin-1-yl)-1,1-dimethylethyl]methanesulfonamide (7.37 g, 18.8 mmol) in DMF. A solution of tert-butyl 6-iodohexylcarbamate (6.75 g, 20.6 mmol) in DMF (approximately 100 mL) was added. The reaction mixture was heated overnight at 65° C. and then concentrated under reduced pressure to provide an orange oil. The oil was partitioned between water (300 mL) and dichloromethane (300 mL). The organic layer was washed sequentially with water (×2) and brine, dried over sodium sulfate, filtered, and then concentrated under reduced pressure. The residue was dissolved in dichloromethane (100 mL), washed sequentially with water (×10) and brine, dried over sodium sulfate, filtered, and then concentrated under reduced pressure to provide 10.85 g of crude product as a yellow foam. The crude product was purified by column chromatography on silica gel (eluting sequentially with 95:5 and 92.5:7.5 dichloromethane:methanol) to provide 8.5 g of tert-butyl{6-[2-ethoxymethyl-1-(2-methanesulfonylamino-2-methylpropyl)-1H-imidazo[4,5-c]quinolin-1-yloxy]hexyl}carbamate as a white solid.

Part K

3-Chloroperoxybenzoic acid (4.23 g Of 60%, 14.4 mmol) was added in a single portion to a solution of tert-butyl{6-[2-ethoxymethyl-1-(2-methanesulfonylamino-2-methylpropyl)-1H-imidazo[4,5-c]quinolin-1-yloxy]hexyl}carbamate (8.5 g, 14.4 mmol) in chloroform (200 mL). The reaction mixture was stirred for several hours and then washed sequentially with 1% sodium carbonate (×2) and brine. The organic layer was dried over sodium sulfate, filtered, and then concentrated under reduced pressure to provide 9.20 g of tert-butyl{6-[2-ethoxymethyl-1-(2-methanesulfonylamino-2-methylpropyl)-5-oxido-1H-imidazo[4,5-c]quinolin-1-yloxy]hexyl}carbamate as a orange solid.

Part L

Ammonium hydroxide (20 mL) and p-toluenesulfonyl chloride (2.74 g, 14.4 mmol) were added sequentially with rapid stirring to a mixture of the material from Part K in dichloromethane (150 mL), and the reaction was stirred for two hours. The organic layer was then washed with saturated aqueous sodium bicarbonate (2×) and brine, dried over sodium sulfate, filtered, and concentrated under reduced pressure to provide tert-butyl{6-[4-amino-2-ethoxymethyl-1-(2-methanesulfonylamino-2-methylpropyl)-1H-imidazo[4,5-c]quinolin-1-yloxy]hexyl}carbamate as a red solid.

Part M

A solution of the material from Part L in hydrochloric acid in ethanol (50 mL of 4.25 M) was heated to reflux and then allowed to cool to ambient temperature. The reaction mixture was purged with nitrogen for approximately 1 hour and then concentrated under reduced pressure. The residue was dissolved in water and then washed with chloroform (×2). The pH of the aqueous layer was adjusted with ammonium hydroxide and then the aqueous layer was extracted with chloroform (×3). The combined extracts were washed with brine, dried over sodium sulfate, filtered, and then concentrated under reduced pressure to provide 6.86 g of N-{2-[4-amino-7-(6-aminohexyloxy)-2-ethoxymethyl-1H-imidazo[4,5-c]quinolin-1-yl]-1,1-dimethylethyl}methanesulfonamide as a tan solid.

### Example 1

An IRM is covalently attached to a polyethylene glycol polymer by the formation of an amide bond. An IRM containing a pendant amine group is reacted with an activated polyethylene glycol polymer containing an N-hydroxysuccinimidyl ester to form an amide bond as shown below.

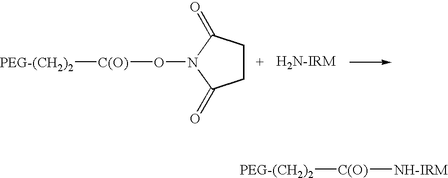

The polyethylene glycol polymer may be linear as shown above or branched as shown below.

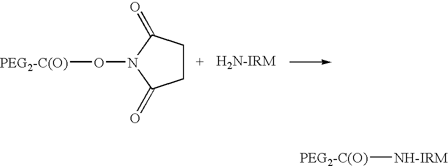

The polyethylene glycol polymer backbone may be difunctional as shown below.
HO—(CH₂CH₂O)ₙ—CH₂CH₂—OH.

Alternatively, the polyethylene glycol polymer backbone may be capped at one end to provide a monofunctional polymer; for example,
CH₃—O—(CH₂CH₂O)ₙ—CH₂CH₂—OH.

IRMs containing pendant amine groups and methods of making them are known. See, for example, U.S. Pat. Nos. 6,451,810; 6,677,349; 6,660,747; 6,545,016; 6,194,425; and 6,069,149; U.S. Patent Publication No. 2004/0010007; and U.S. Patent Publication Nos. 2004/0147543 and 2004/0176367.

Some activated polyethylene glycol polymers containing N-hydroxysuccinimidyl ester groups are commercially available; for example, those available from Nektar, San Carlos, Calif. Others can be prepared using known synthetic methods. See, for example, U.S. Pat. No. 5,583,114 and the references cited therein.

### Example 2

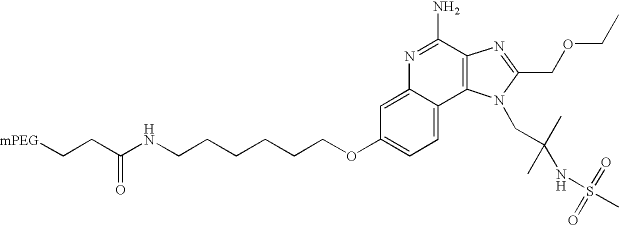

N-{2-[4-Amino-7-(6-aminohexyloxy)-2-ethoxymethyl-1H-imidazo[4,5-c]quinolin-1-yl]-1,1-dimethylethyl}methanesulfonamide is reacted with mPEG-succinimidyl propionate having a molecular weight of 2,000 Da (available as mPEG-SPA, MW 2,000 Da, from Nektar). mPEG is a monofunctional polymer having one end capped with a methoxy group.

### Example 3

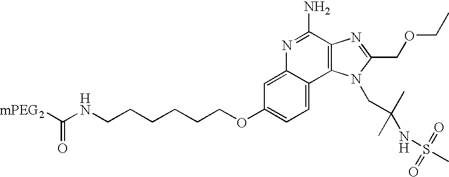

N-{2-[4-Amino-7-(6-aminohexyloxy)-2-ethoxymethyl-1H-imidazo[4,5-c]quinolin-1-yl]-1,1-dimethylethyl}methanesulfonamide is reacted with mPEG₂-N-Hydroxysuccinimide having a molecular weight of 40 kDa (available as mPEG₂-NHS, MW 40 kDa, from Nektar).

### Example 4

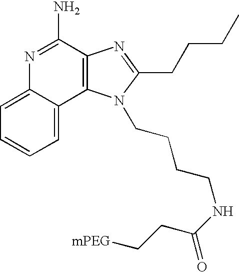

1-(4-Aminobutyl)-2-butyl-1H-imidazo[4,5-c]quinolin-4-amine (which can be prepared according to the methods of U.S. Pat. No. 6,069,149) is reacted with mPEG-succinimidyl propionate having a molecular weight of 2,000 Da (available as mPEG-SPA, MW 2,000 Da, from Nektar).

### Example 5

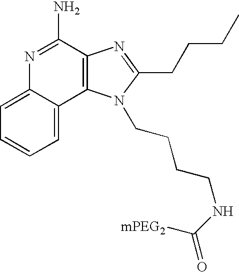

1-(4-Aminobutyl)-2-butyl-1H-imidazo[4,5-c]quinolin-4-amine is reacted with mPEG₂-N-Hydroxysuccinimide having a molecular weight of 40 kDa (available as mPEG₂-NHS, MW 40 kDa, from Nektar).

### Example 6

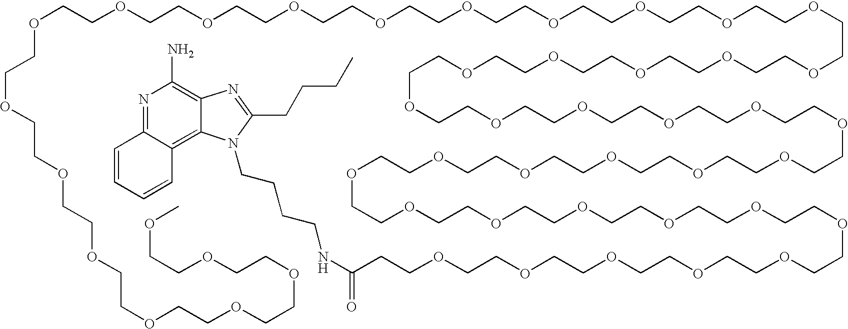

1-(4-Aminobutyl)-2-butyl-1H-imidazo[4,5-c]quinolin-4-amine (31 mg, 0.10 mmol, which can be prepared according to the methods disclosed in U.S. Pat. No. 6,069,149) was dissolved in dichloromethane (5 mL). The solution was stirred under a nitrogen atmosphere and cooled to 0° C. mPEG-succinimidyl propionate having a molecular weight of 2,000 Da (200 mg, available as mPEG-SPA, MW 2,000 Da, from Nektar) was then added and the reaction was stirred overnight. The reaction mixture was then concentrated and applied to a silica gel column (2×10 cm). Elution with 33% CMA (CMA=80:18:2 v:v:v chloroform/methanol/concentrated ammonium hydroxide) in chloroform gave the desired product as a colorless syrup. Repeated concentration from diethyl ether gave 165 mg of product as a white solid, mp 48-49.5° C. Mass spectral analysis showed a bell-shaped distribution of pegylated products centered at about m/z 2380. This corresponds to 45 ethylene oxide units in the PEG chain. ¹H NMR (300 MHz, CDCl₃) δ 7.91 (d, J=8.1 Hz, 1H), 7.80 (d, J=8.4 Hz, 1H), 7.49 (m, 1H), 7.31 (m, 1H), 6.72 (m, 1H), 5.39 (s, 2H), 4.48 (t, J=7.6 Hz, 2H), 2.91 (t, J=7.8 Hz, 2H), 2.43 (t, J=5.6 Hz, 2H), 1.98-1.75 (m, 4H), 1.67 (m, 2H), 1.54 (m, 2H), 1.01 (t, J=7.3 Hz, 3H); QTOF-MS(ESI) m/z 2248, 2292, 2336, 2380 (C₁₁₂H₂₁₁N₅O₄₇), 2424, 2468, 2512, 2556, 2600, 2644, 2688, 2732.

### Example 7

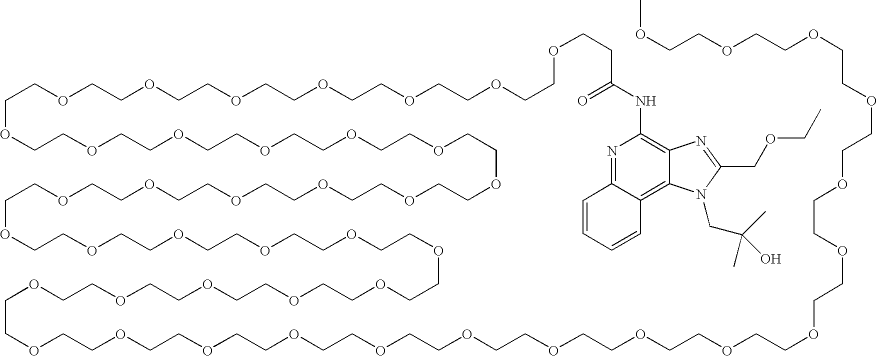

4-Amino-2-ethoxymethyl-α,α-dimethyl-1H-imidazo[4,5-c]quinoline-1-ethanol (resiquimod, 31 mg, 0.10 mmol, which can be prepared as described in Example 99 of U.S. Pat. No. 5,389,640) was dissolved in tetrahydrofuran (5 mL). The solution was stirred under a nitrogen atmosphere. mPEG-succinimidyl propionate having a molecular weight of 2,000 Da (231 mg, available as MPEG-SPA, MW 2,000 Da, from Nektar) was added. After 24 hours, 4-dimethylaminopyridine (5 mg) was added and the reaction was stirred for 7 days. The reaction mixture was then concentrated and applied to a silica gel column (2.5×10 cm). Elution with 3% methanol in chloroform (saturated with ammonium hydroxide), followed by 5% methanol in chloroform (saturated with ammonium hydroxide), and 10% methanol in chloroform (saturated with ammonium hydroxide) gave the desired product as a colorless syrup. Repeated chromatography using the same conditions gave pure material. Concentration from diethyl ether gave 110 mg of product as a white solid, mp 51-52° C. Mass spectral analysis showed a bell-shaped distribution of pegylated products centered at about m/z 2383. This corresponds to 45 ethylene oxide units in the PEG chain. ¹H NMR (300 MHz, CDCl₃) δ 8.30 (d, J=8.2 Hz, 1H), 8.11 (d, J=7.4 Hz, 1H), 7.67 (m, 1H), 7.56 (t, J=7.3 Hz, 1H), 4.98 (s, 2H), 4.83 (s, 2H), 2.50 (t, J=5.5 Hz, 1H), 1.34 (s, 6H), 1.27 (t, J=7.0 Hz, 3H). QTOF-MS(ESI) m/z 2207, 2251, 2295, 2339, 2383 (C₁₁₁H₂₀₈N₄O₄₉), 2427, 2471, 2515, 2559, 2603, 2647, 2691, 2735.

### Example 8

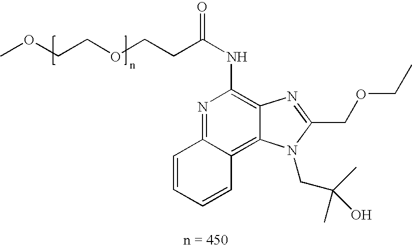

Under a nitrogen atmosphere, 4-amino-2-ethoxymethyl-α,α-dimethyl-1H-imidazo[4,5-c]quinoline-1-ethanol (resiquimod, 15 mg, 0.48 mmol) was dissolved in tetrahydrofaran (5 mL). mPEG-succinimidyl propionate having a molecular weight of 20,000 Da (1.00 g, available as mPEG-SPA, MW 20 kDa, from Nektar) was added and the resulting thick suspension was stirred at ambient temperature over night. The reaction mixture was then heated to 50° C. and everything went into solution. The solution was heated at 50° C. for 7 days and then concentrated under reduced pressure. The residue was dissolved in hot isopropanol (10 mL), the solution was allowed to cool to ambient temperature, and a solid was isolated by filtration. This procedure was repeated to provide about 1.0 g of a white solid.

### Example 9

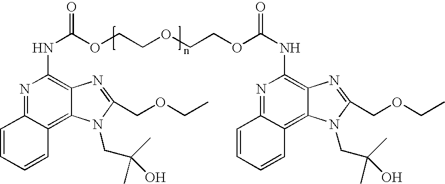

An IRM substituted polyethylene glycol polymer was prepared using the method described in Reaction Scheme III above. A mixture of polyethylene glycol polymer (20 g, 1.0 eq, average Mₙ about 35,000) and toluene (80 mL) was heated to 44° C. Phosgene (20% in toluene, 0.71 g, 2.5 eq) was added. Analysis of a small sample of the reaction mixture by infrared spectroscopy showed a band at 1780 cm⁻¹. The reaction mixture was heated at reflux to drive off the excess phosgene and then cooled back down to 44° C. Triethylamine (121 mg, 2.1 eq) and pentafluorophenol (221 mg, 2.1 eq) were added. Analysis of a small sample of the reaction mixture by infrared spectroscopy showed a band at 1785 cm⁻¹. The reaction mixture was concentrated under reduced pressure. The residue was combined with isopropanol (80 mL, dried over molecular sieves) and 4-amino-2-ethoxymethyl-α,α-dimethyl-1H-imidazo[4,5-c]quinoline-1-ethanol (resiquimod, 367 mg, 2.05 eq). The reaction mixture was heated at reflux for 6 hours and the clear solution was allowed to cool to ambient temperature overnight during which time the reaction mixture solidified. The reaction mixture was warmed until mobile and then poured into isopropanol (about 800 mL). The resulting solid was isolated by filtration and dried to provide 18.9 g of polyethylene glycol polymer end capped with resiquimod.

### Examples 10-12

The IRM compounds used in the Examples provided below are identified in Table 1.

TABLE 1

| Compound | Chemical Name | Reference |
|---|---|---|
| IRM 1 | 4-amino-α,α-dimethyl-2-ethoxy- | U.S. Pat. No. |
| | methyl-1H-imidazo[4,5-c]quinolin- | 5,389,640 |
| | 1-ethanol | Example 99 |
| IRM 2 | N-[4-(4-Amino-2-butyl-1H-imidazo[4,5- | U.S. Pat. No. |
| | c]quinolin-1-yl)butyl]acetamide | 6,451,810# |

#This compound is not specifically exemplified but can be readily prepared using the synthetic methods disclosed in the cited reference.

### Example 10

An in vitro human blood cell system is used to assess cytokine induction. Activity is based on the measurement of interferon (α) and tumor necrosis factor (α) (IFN-α and TNF-α, respectively) secreted into culture media as described by Testerman et. al. in “Cytokine Induction by the Immunomodulators Imiquimod and S-27609,” Journal of Leukocyte Biology, 58, 365-372 (September, 1995).

Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Blood is diluted 1:1 with Dulbecco's Phosphate Buffered Saline (DPBS) or Hank's Balanced Salts Solution (HBSS). Alternately, whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete. The PBMC suspension is added to 96 well flat bottom sterile tissue culture plates containing an equal volume of RPMI complete media containing test compound.

Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM. Controls include cell samples with media only, cell samples with DMSO only (no compound), and cell samples with reference compound.

Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (usually 30-0.014 μM). The final concentration of PBMC suspension is 2×10⁶ cells/mL. The plates are covered with sterile plastic lids, mixed gently and then incubated for 18 to 24 hours at 37° C. in a 5% carbon dioxide atmosphere.

Separation

Following incubation the plates are centrifuged for 10 minutes at 1000 rpm (approximately 200×g) at 4° C. The cell-free culture supernatant is removed and transferred to sterile polypropylene tubes. Samples are maintained at −30 to −70° C. until analysis. The samples are analyzed for IFN-α by ELISA and for TNF-α by IGEN/BioVeris Assay.

Interferon (α) and Tumor Necrosis Factor (α) Analysis

IFN-α concentration is determined with a human multi-subtype calorimetric sandwich ELISA (Catalog Number 41105) from PBL Biomedical Laboratories, Piscataway, N.J. Results are expressed in pg/mL.

The TNF-α concentration is determined by ORIGEN M-Series Immunoassay and read on an IGEN M-8 analyzer from BioVeris Corporation, formerly known as IGEN International, Gaithersburg, Md. The immunoassay uses a human TNF-α capture and detection antibody pair (Catalog Numbers AHC3419 and AHC3712) from Biosource International, Camarillo, Calif. Results are expressed in pg/mL.

Assay Data and Analysis

In total, the data output of the assay consists of concentration values of TNF-α and IFN-α (y-axis) as a function of compound concentration (x-axis).

Analysis of the data has two steps. First, the greater of the mean DMSO (DMSO control wells) or the experimental background (usually 20 pg/mL for IFN-α and 40 pg/mL for TNF-α) is subtracted from each reading. If any negative values result from background subtraction, the reading is reported as

“*”, and is noted as not reliably detectable. In subsequent calculations and statistics, “* ”, is treated as a zero. Second, all background subtracted values are multiplied by a single adjustment ratio to decrease experiment to experiment variability. The adjustment ratio is the area of the reference compound in the new experiment divided by the expected area of the reference compound based on the past 61 experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from the past 61 experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response (pg/mL) is the maximal response attained in the dose response curve. Results are shown in Table 2.

TABLE 2

Minimum Effective Concentration (mM)

| Compound | IFN-α | TNF |
|---|---|---|
| IRM 1 | 0.12 | 0.37 |
| IRM 2 | 0.014 | 1.11 |
| Example 6 | 30 | >30 |
| Example 7 | 3.33 | 30 |

### Example 11

Example 7 and Example 8 were prepared at 0.1 and 1.0 mg/ml, respectively, in either citrate buffered saline at pH 4 or phosphate buffered saline at pH 7.4. Samples were placed in a thermostated autosampler with the temperature controlled at 37° C. Samples were injected periodically over the course of the experiment and the % of IRM1 liberated was measured by an HPLC system equipped with a thermostatted autosampler set at 37° C. and a Zorbax SB C18, (3.0×150 mm), 3.5 μm particle size column with a column temperature of 40° C. Samples were eluted with a mobile phase of 1% acetic acid in water and methanol. The mobile phase was run at a ratio of 55:45 of 1% acetic acid in water to methanol for five minutes, gradient to 5:95 for ten minutes, held at 5:95 for five minutes, gradient to 55:45 in less than a minute, and held at 55:45 for ten minutes. All HPLC runs were set at a flow rate of 0.5 mL/min, 20 μL injection volume, and a 254 nm UV detection wavelength. The % IRM1 was determined by normalizing the IRM1 peak area by the total peak area of the chromatogram. Results for Example 7 and Example 8 are shown in Tables 3 and 4, respectively.

TABLE 3

| | |
|---|---|
| Citrate Buffered Saline, | Phosphate Buffered Saline, |
| pH 4, 37° C. | pH 7.4, 37° C. |

| Time (hr) | % IRM1 | Time (hr) | % IRM1 |
|---|---|---|---|
| 0 | 2.9% | 1 | 1.3% |
| 6 | 5.7% | 5 | 1.5% |
| 10 | 7.4% | 10 | 1.7% |
| 13 | 9.4% | 13 | 1.8% |
| 17 | 10.6% | 18 | 1.9% |
| 20 | 12.1% | 20 | 2.1% |
| 23 | 13.0% | 23 | 2.1% |
| 28 | 16.5% | 27 | 2.3% |
| 30 | 17.1% | 30 | 2.4% |

TABLE 4

| | |
|---|---|
| Citrate Buffered Saline, | Phosphate Buffered Saline, |
| pH 4, 37° C. | pH 7.4, 37° C. |

| Time (hr) | % IRM1 | Time (hr) | % IRM1 |
|---|---|---|---|
| 0 | NM | 0 | NM |
| 6 | 6.2% | 7 | 3.6% |
| 10 | 7.7% | 11 | 3.7% |
| 13 | 9.2% | 14 | 3.8% |
| 17 | 11.0% | 18 | 4.0% |
| 20 | 12.6% | 21 | 4.1% |
| 23 | 14.1% | 24 | 4.2% |
| 28 | 16.0% | 29 | 4.4% |
| 31 | 17.7% | 32 | 4.5% |

NM = Not Measured

### Example 12

The solubility of IRM2 and the IRM-polymer complex exemplified in Example 6 was determined in normal saline and phosphate buffered saline (PBS) at pH 7.4. Each compound was added to each medium until saturation had been reached. Vials containing the saturated solutions were capped and placed into a shaking water bath at 25° C. After 7 days the saturated solutions were filtered and analyzed for compound content on an HPLC using a Zorbax Bonus-RP 150×4.6 mm 5 μm particle size column. IRM2 was eluted with a 25:75 ratio of 0.05% trifuoro-acetic acid (TFA) in Acetonitrile to 0.1% TFA in water. Example 6 was eluted with a 10:90 ratio of 0.05% TFA in Acetonitrile to 0.1% TFA in water for three minutes, gradient to a 75:25 ratio of 0.05% TFA in Acetonitrile to 0.1% TFA in water for seven minutes and held at the 75:25 ratio for eight minutes. All HPLC runs were set at a flow rate of 1 mL/min, 20 μL injection volume, and a 254 nm UV detection wavelength. Quantitation was performed against external standards. Results are shown in Table 5 expressed in millimolar (mM) and solubility fold increase of Example 6 over IRM2.

TABLE 5

| | Solubility | | |
|---|---|---|---|
| Aqueous System | IRM2 (mM) | Example 6 (mM) | Fold Increase |
| Saline | 0.04 | 5.97 | 142.50 |
| Phosphate Buffered | 0.07 | 5.59 | 75.97 |
| Saline, pH 7.4 | | | |

The complete disclosures of the patents, patent documents and publications cited herein are incorporated by reference in their entirety as if each were individually incorporated. In case of conflict, the present specification, including definitions, shall control. Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of delivering one or more IRM compounds to a tissue in a subject, the method comprising administering an IRM preparation to the subject, wherein the IRM preparation comprises a soluble IRM-polymer complex comprising one or more IRM compounds attached to a polymer.

2. A method of delivering one or more IRM compounds to a tissue in a subject, the method comprising administering an IRM preparation to the subject, wherein the IRM preparation comprises a soluble IRM-polymer complex comprising one or more IRM compounds attached to a soluble polymer comprising alkylene oxide moieties, wherein the IRM-polymer complex has a molecular weight of 1 kDa to 500 kDa.

3. (canceled)

4. The method of claim 1 wherein the soluble IRM-polymer complex has a solubility of at least 0.1 microgram per milliliter in water under physiological conditions.

5. (canceled)

6. The method of claim 1 wherein one or more IRM compounds are covalently attached to a soluble polymer.

7.-13. (canceled)

14. The method of claim 1 wherein the tissue is a tumor.

15. The method of claim 14 wherein the tumor is a breast cancer tumor, a stomach cancer tumor, a lung cancer tumor, a head or neck cancer tumor, a colorectal cancer tumor, a renal cell carcinoma tumor, a pancreatic cancer tumor, a basal cell carcinoma tumor, a cervical cancer tumor, a melanoma cancer tumor, a prostate cancer tumor, an ovarian cancer tumor, or a bladder cancer tumor.

16. (canceled)

17. The method of claim 1 wherein the polymer comprises alkylene oxide moieties.

18. The method claim 1 wherein the IRM is an agonist of at least one TLR selected from the group consisting of TLR7 and TLR8.

19.-26. (canceled)

27. The method of claim 1 wherein the IRM compound is selected from the group consisting of imidazoquinoline amines; tetrahydroimidazoquinoline amines; and imidazopyridine amines; 1,2-bridged imidazoquinoline amines; 6,7-fused cycloalkylimidazopyridine amines; imidazonaphthyridine amines, tetrahydroimidazonaphthyridine amines; oxazoloquinoline amines; thiazoloquinoline amines oxazolopyridine amines; thiazolopyridine amines; oxazolonaphthyridine amines; thiazolonaphthyridine amines; 1H-imidazo dimers fused to pyridine amines, quinoline amines, tetrahydroquinoline amines, naphthyridine amines, or tetrahydronaphthyridine amines; and combinations thereof.

28. The method of claim 1 wherein the IRM compound is selected from the group consisting of purines, imidazoquinoline amides, benzimidazoles, 1H-imidazopyridines, adenines, and derivatives thereof.

29.-31. (canceled)

32. The method of claim 1 wherein the polymer is a soluble polymer selected from the group consisting of poly(alkylene glycols), poly(olefinic alcohols), polyvinylpyrrolidones, poly(hydroxyalkylmethacrylamides), poly(hydroxyalkylmethacrylates), polyvinyl alcolhols, polyoxazolines, poly(acrylic acids), polyacrylamides, polyglutamates, polylysines, polysaccharides, and combinations thereof.

33. A soluble IRM-polymer complex comprising one or more IRM compounds attached to an alkylene oxide-containing polymer.

34.-39. (canceled)

40. The soluble IRM-polymer complex of claim 33 wherein the soluble IRM-polymer complex has a solubility of at least 0.1 microgram per milliliter in water under physiological conditions.

41. (canceled)

42. The soluble IRM-polymer complex of claim 33 wherein the IRM-polymer complex has a molecular weight of 1 kDa to 500 kDa.

43. (canceled)

44. (canceled)

45. The soluble IRM-polymer complex of claim 42 wherein the IRM-polymer complex has a molecular weight of 20 kDa to 200 kDa.

46. (canceled)

47. The soluble IRM-polymer complex of claim 33 wherein the one or more IRM compounds are covalently attached to an alkylene oxide-containing polymer.

48. The soluble IRM-polymer complex of claim 42 wherein the soluble polymer is selected from the group consisting of poly(alkylene glycols), poly(olefinic alcohols), polyvinylpyrrolidones, poly(hydroxyalkylmethacrylamides), poly(hydroxyalkylmethacrylates), polyvinyl alcohols, polyoxazolines, poly(acrylic acids), polyacrylamides, polyglutamates, polylysines, polysaccharides and combinations thereof.

49.-52. (canceled)
